(19) United States
(12) Patent Application Publication
(10) Pub. No.: US 2006/0223059 A1
(43) Pub. Date: Oct. 5, 2006

(54) Methods for detecting endocrine cancer

(75) Inventors: George Yousef, St. John's, Newfoundland & Labrador (CA); Eleftherios P. Diamandis, Toronto (CA)

Correspondence Address: MERCHANT & GOULD PC, P.O. BOX 2903, MINNEAPOLIS, MN, 55402-0903, US

(73) Assignee: Mount Sinai Hospital, Toronto, ON (CA)

(21) Appl. No.: 10/529,163

(22) PCT Filed: Sep. 26, 2003
(86) PCT No.: PCT/CA03/01478

§ 371 Date: Dec. 19, 2005

Related U.S. Application Data
(60) Provisional application No. 60/414,107, filed on Sep. 26, 2002.
(60) Provisional application No. 60/450,928, filed on Feb. 27, 2003.

(51) Int. Cl. C12Q 1/68; G01N 33/574

(52) U.S. Cl. 435/6; 435/7.23

(57) ABSTRACT

Kallikrein 12, kallikrein 14, and kallikrein 15 proteins, and nucleic acids encoding the proteins have particular application in the detection of endocrine cancer, in particular ovarian cancer. Kallikrein 12, kallikrein 14, and kallikrein 15 proteins and nucleic acid molecules encoding the proteins constitute new biomarkers for diagnosis and monitoring (i.e. monitoring progression or therapeutic treatment) of endocrine cancer.

### FIELD OF THE INVENTION

The invention relates to methods for detecting endocrine cancer.

### BACKGROUND OF THE INVENTION

Epithelial ovarian carcinoma is the most common and most lethal of all gynecologic malignancies. Only 30% of ovarian tumors are diagnosed at an early stage (Stage I/II), when survival rates reach 90%. The rest are diagnosed at an advanced stage, with survival rates of less than 20% (Greenlee R T, Hill-Harmon M B, Murray T, et al., 2001. CA Cancer J Clin. 2001;51:15-36). Currently, the only well-accepted serological marker is CA125, a large glycoprotein of unknown function (Meyer T, Rustin G J., Br J Cancer. 2000;82:1535-1538). CA125 has serious limitations as a diagnostic, prognostic and screening tool (Holschneider C H, Berek J S, Semin Surg Oncol. 2000;19:3-10). Consequently, there is a need to develop new biomarkers which can assist in the prognosis and progression of this malignancy, in reaching treatment decisions, in monitoring response after treatment and for identifying relapse during routine follow-up. Several putative markers have been sought to compensate for the limitations of CA125, including inhibin, prostasin, OVX1, LASA, CA15.3 and CA72-4 (Lambert-Messerlian G M., Eur J Endocrinol. 2000;142:331-333; Mok S C, et al., J Natl Cancer Inst. 2001;93: 1458-1464; Xu F J, et al., J Clin Oncol. 1993;11:1506-1510; Patsner B, et al., Gynecol Oncol. 1988;30:98-103; Woolas R P, et al., Gynecol Oncol. 1995;59:111-116; and Negishi Y, et al., Gynecol Oncol. 1993;48: 148-154). Although their relevance in the management of ovarian carcinoma is yet to be determined, these novel markers may by used in combination with CA125, to enhance the overall diagnostic/prognostic capability (Menon U, Jacobs I J, Curr Opin Obstet Gynecol. 2000;12:39-42).

Kallikreins are a subgroup of secreted serine proteases, encoded by highly conserved and tightly clustered multigene families in humans, rats and mice. The human kallikrein gene family resides on chromosome 19q13.4 and is comprised of 15 members, whose genes are designated as KLK1 to KLK15 and the corresponding proteins as hK1 to hK15 (Yousef G M, Diamandis E P., Endocr Rev. 2001;22:184-2041; Yousef G M, et al., Biochem Biophys Res Commun. 2000;276:125-133; Diamandis E P, et al. Clin Chem. 2000;46: 1855-1858). Kallikreins are expressed in a wide variety of tissues and are found in many biological fluids (e.g. cerebrospinal fluid, serum, seminal plasma, milk, etc.) where they are predicted to process specific substrates. Kallikreins may participate in cascade reactions similar to those involved-in digestion, fibrinolysis, coagulation, wound healing and apoptosis (Yousef G M, Diamandis E P., Endocr Rev. 2001;22:184-2041). Many kallikreins have been found to be differentially expressed in endocrine-related malignancies (Diamandis E P, Yousef G M, Expert Rev. Mol. Diagn. 2001;1:182-190), including prostate (Barry M J. Clinical practice, N Engl J Med. 2001;344:1373-1377; Rittenhouse H G, et al., Crit Rev Clin Lab Sci. 1998;35:275-368; and Yousef G M, et al., J Biol Chem. 2001;276:53-61, ovarian (Kim H, et al., Br J Cancer, 2001;84:643-650; Anisowicz A, et al., Mol Med. 1996;2:624-636; Tanimoto H, et al., Cancer. 1999;86:2074-2082; Magklara A, et al., Clin Cancer Res. 2001;7:806-811; Yousef G M, et al., Cancer Res. 2001;61:7811-7818; Luo L, et al Clin Chim Acta. 2001;306:111-118), breast (Yousef G M, et al., Cancer Res. 2001;61:3425-3431; Yousef G M, et al, J Biol Chem. 2000;275:11891-11898; and Yousef G M, et al, Genomics. 2000;69:331-341), and testicular cancer (Luo L Y, et al.,2001;85:220-224). In addition, many kallikrein genes examined thus far are under steroid hormone regulation, implicating a role for kallikreins in endrocrine-related tissues (Yousef G M, Diamandis E P., Endocr Rev., 2001;22:184-204). Furthermore, hK6, hK10 and hK11 have been recently identified as novel serological ovarian cancer biomarkers (Luo L, et al, Clin Chim Acta. 2001;306:111-118 Diamandis E P, et al., Clin Biochem. 2000;33:579-583, and Diamandis E P, et al., Cancer Res. 2002;62:295-300).

KLK12 has recently been cloned (Yousef G M et al, Genomics 2000 Nov. 1;69(3):331-41). KLK12 is expressed in a variety of tissues including salivary gland, stomach, uterus, lung, thymus, prostate, colon, brain, breast, thyroid, and trachea. Preliminary work suggests that the expression of KLK12 is down regulated at the mRNA level in breast cancer tissues and is up-regulated by steroid hormones in breast and prostate cancer cell lines.

KLK14 (formerly known as KLK-L6) has also recently been cloned (Yousef G M, et al. Cancer Res 2001;61:3425-3431). This gene has a restricted tissue expression pattern and is found in the central nervous system, particularly the brain, cerebellum and spinal cord, as well as in endocrine-related tissues such as the uterus, ovary, thyroid and testis. Preliminary studies have shown that KLK14 is down-regulated at the mRNA level in prostatic, testicular, ovarian and breast cancer tissues (compared to normal tissues) and two breast cancer cell lines (Yousef G M, et al. Cancer Res. 2001;61:3425-3431). In this respect, KLK14 resembles KLK3 (PSA) and KLK10 in breast cancer and KLK9 in ovarian cancer (Yousef G M, et al., Cancer Res. 2001;61:7811-7818. Yu H, et al., Cancer Res. 1995;55:2104-2110, and Dhar S, et al., Clin Cancer Res. 2001;7:3393-3398). In situ hybridization studies demonstrated that KLK14 is expressed by the secretory epithelial cells of benign prostate gland, prostatic intraepithelial neoplasia and malignant prostate cells (Hooper J D, et al., Genomics. 2001;73:117-122).

KLK15 (encoding for hK15, a protein also named “prostinogen”) is the most recently cloned member of the human kallikrein gene family (Yousef G M, et al., J Biol Chem 276:53-61, 2001; Takayama T K, et al., Biochemistry 40:1679-1687, 2001). It is formed of 5 coding exons and encodes for a serine protease of a predicted molecular weight of about 28 kDa. KLK15 shares a high degree of structural similarity with KLK3 (also known as prostate specific antigen, PSA) and other kallikreins. Similarly to KLK3, but unlike other trypsin-like serine proteases, KLK15 does not have an aspartate residue in the substrate-binding pocket, suggesting a chymotrypsin-like substrate specificity. Preliminary work has shown that KLK15 is up-regulated, at the mRNA level in prostate cancer (Yousef G M, Scorilas A, Jung K, et al., J Biol Chem 276:53-61, 2001). A recent report indicated that hK15 can readily activate the precursor of PSA by cleaving an amino terminal peptide bond (Takayama T K, et al Biochemistry 40:1679-1687, 2001). In addition, KLK15 has been shown to be under steroid hormone regulation, possibly through the androgen receptor (AR).

The citation of any reference herein is not an admission that such reference is available as prior art to the instant invention.

### SUMMARY OF THE INVENTION

Briefly stated the present invention relates to novel biomarkers for endocrine cancer, in particular ovarian, breast and prostate cancer, more particularly ovarian cancer. The invention provides compositions and methods for diagnosing endocrine cancer.

Kallikrein 12, kallikrein 14, and kallikrein 15 proteins and nucleic acids encoding the proteins have particular application in the detection of endocrine cancer, in particular ovarian cancer. Kallikrein 12, kallikrein 14, and kallikrein 15 proteins and nucleic acids encoding the proteins constitute new biomarkers for diagnosis and monitoring (i.e. monitoring progression or therapeutic treatment) of endocrine cancer. In accordance with an aspect of the invention these proteins and nucleic acids are used for the diagnosis, monitoring, progression, treatment, and prognosis of endocrine cancer, and they may be used as biomarkers before surgery or after relapse.

Kallikrein 12, kallikrein 14, and/or kallikrein 15 proteins, nucleic acids encoding the proteins, and agents that bind to the proteins, may be used to detect endocrine cancer and they can be used in the diagnostic evaluation of endocrine cancer, and the identification of subjects with a predisposition to such disorder.

In accordance with the methods of the invention, kallikrein 12, kallikrein 14, and/or kallikrein 15 can be assessed, for example by detecting the presence in the sample of (a) a polypeptide or polypeptide fragment corresponding to the marker; (b) a metabolite which is produced directly or indirectly by a polypeptide corresponding to the marker, (c) a transcribed nucleic acid or fragment thereof having at least a portion with which the marker is substantially identical; and/or (c) a transcribed nucleic acid or fragment thereof, wherein the nucleic acid hybridizes with the marker.

In an embodiment of the invention, a method is provided for detecting kallikrein 12, kallikrein 14, and/or kallikrein 15 or KLK12, KLK14 and/or KLK15 associated with endocrine cancer in a patient comprising:
(a) taking a sample from a patient;
(b) detecting or identifying in the sample kallikrein 12, kallikrein 14, and/or kallikrein 15 or KLK12, KLK14 and/or KLK15; and
(c) comparing detected amounts with amounts detected for a standard.

The term “detect” or “detecting” includes assaying, imaging or otherwise establishing the presence or absence of the target kallikrein 12, kallikrein 14, and/or kallikrein 15 proteins or nucleic acids encoding the proteins, subunits thereof, or combinations of reagent bound targets, and the like, or assaying for, imaging, ascertaining, establishing, or otherwise determining one or more factual characteristics of endocrine cancer, metastasis, stage, or similar conditions. The term encompasses diagnostic, prognostic, and monitoring applications for the kallikrein 12, kallikrein 14, and/or kallikrein 15 proteins and nucleic acid molecules encoding the proteins.

Thus, the invention provides a method of assessing whether a patient is afflicted with or has a pre-disposition for endocrine cancer, the method comprising comparing:
(a) levels of kallikrein 12, kallikrein 14 and/or kallikrein 15 or nucleic acids encoding kallikrein 12, kallikrein 14 and/or kallikrein 15 in a sample from the patient; and
(b) levels of kallikrein 12, kallikrein 14 and/or kallikrein 15 or nucleic acids encoding kallikrein 12, kallikrein 14 and/or kallikrein 15 in samples of the same type obtained from control patients not afflicted with endocrine cancer, wherein significantly altered levels of kallikrein 12, kallikrein 14 and/or kallikrein 15 or nucleic acids encoding kallikrein 12, kallikrein 14 and/or kallikrein 15 relative to levels for control subjects of kallikrein 12, kallikrein 14 and/or kallikrein or nucleic acids encoding kallikrein 12, kallikrein 14 and/or kallikrein 15 is an indication that the patient is afflicted with endocrine cancer.

In a particular embodiment of a method of the invention for assessing whether a patient is afflicted with or has a pre-disposition for breast, ovarian, or prostate cancer, in particular ovarian or breast cancer, higher levels of kallikrein 14 in a sample relative to the control subject levels is an indication that the patient is afflicted with breast, ovarian, or prostate cancer, in particular ovarian or breast cancer.

In another particular embodiment of a method of assessing whether a patient is afflicted with endocrine cancer, in particular ovarian or breast cancer (e.g. screening, detection of a recurrence, reflex testing), the method comprises comparing:
(a) levels of kallikrein 14; and
(b) normal levels of kallikrein 14 in a control non-cancer sample.

A significant difference between the levels of kallikrein 14 in the patient sample and the normal levels (e.g. higher in the patient samples) is an indication that the patient is afflicted with endocrine cancer.

In an aspect, the invention provides a method for monitoring the progression of endocrine cancer in a patient the method comprising:
(a) detecting kallikrein 12, kallikrein 14, and/or kallikrein 15 proteins or nucleic acids encoding the proteins in a sample from the patient at a first time point;
(b) repeating step (a) at a subsequent point in time; and
(c) comparing the levels detected in (a) and (b), and therefrom monitoring the progression of the endocrine cancer.

In another aspect, the invention provides a method for assessing the aggressiveness or indolence of a cancer (e.g. staging), the method comprising comparing:
(a) levels of kallikrein 12, kallikrein 14 and/or kallikrein 15 or nucleic acids encoding kallikrein 12, kallikrein 14 and/or kallikrein 15 in a patient sample; and
(b) levels of kallikrein 12, kallikrein 14 and/or kallikrein 15 or nucleic acids encoding kallikrein 12, kallikrein 14 and/or kallikrein 15 in a control sample.

A significant difference between the levels in the sample and the levels in a control sample (e.g. normal or benign) is an indication that the endocrine cancer is aggressive or indolent. In an embodiment, the levels of kallikrein 14 are higher than normal levels.

The invention provides a method for determining whether an endocrine cancer has metastasized or is likely to metastasize in the future, the method comprising comparing:
(a) levels of kallikrein 12, kallikrein 14 and/or kallikrein 15 or nucleic acids encoding kallikrein 12, kallikrein 14 and/or kallikrein 15 in a patient sample; and
(b) normal levels (or non-metastatic levels) of kallikrein 12, kallikrein 14 and/or kallikrein 15 or nucleic acids encoding kallikrein 12, kallikrein 14 and/or kallikrein 15 in a control sample.

A significant difference between the levels in the patient sample and the normal levels is an indication that the endocrine cancer has metastasized or is likely to metastasize in the future.

The invention also provides a method for assessing the potential efficacy of a test agent for inhibiting endocrine cancer, and a method of selecting an agent for inhibiting endocrine cancer.

The invention also contemplates a method of assessing the potential of a test compound to contribute to endocrine cancer comprising:
(a) maintaining separate aliquots of endocrine cancer diseased cells in the presence and absence of the test compound; and
(b) comparing levels of kallikrein 12, kallikrein 14, and/or kallikrein 15 proteins or nucleic acids encoding the proteins in each of the aliquots.

A significant difference between the levels of kallikrein 12, kallikrein 14, and/or kallikrein 15 or nucleic acids encoding the proteins in an aliquot maintained in the presence of (or exposed to) the test compound relative to the aliquot maintained in the absence of the test compound, indicates that the test compound potentially contributes to endocrine cancer.

The invention further relates to a method of assessing the efficacy of a therapy for inhibiting endocrine cancer in a patient. A method of the invention comprises comparing: (a) levels of kallikrein 12, kallikrein 14, and/or kallikrein 15 proteins or nucleic acids encoding the proteins in a sample from the patient obtained from the patient prior to providing at least a portion of the therapy to the patient; and (b) levels of kallikrein 12, kallikrein 14, and/or kallikrein 15 or nucleic acids encoding the proteins in a second sample obtained from the patient following therapy.

A significant difference between the levels of kallikrein 12, kallikrein 14, and/or kallikrein 15 or nucleic acids encoding the proteins in the second sample relative to the first sample is an indication that the therapy is efficacious for inhibiting endocrine cancer.

In an embodiment, the method is used to assess the efficacy of a therapy for inhibiting breast, ovarian, or prostate cancer, in particular ovarian or breast cancer, where lower levels of kallikrein 14 in the second sample relative to the first sample, is an indication that the therapy is efficacious for inhibiting the cancer.

The “therapy” may be any therapy for treating endocrine cancer including but not limited to therapeutics, radiation, immunotherapy, gene therapy, and surgical removal of tissue. Therefore, the method can be used to evaluate a patient before, during, and after therapy.

In an aspect, the present invention relates to a method for diagnosing and monitoring endocrine carcinoma in a subject comprising measuring kallikrein 12, kallikrein 14, and/or kallikrein 15 in a sample from the subject.

In an embodiment of the invention, a method for screening a subject for endocrine cancer is provided comprising (a) obtaining a biological sample from a subject; (b) detecting the amount of kallikrein 12, kallikrein 14, and/or kallikrein 15 in said sample; and (c) comparing said amount of kallikrein 12, kallikrein 14, and/or kallikrein 15 detected to a predetermined standard, where detection of a level of kallikrein 12, kallikrein 14, and/or kallikrein 15 that differs significantly from the standard indicates disease.

A significant difference between the levels of kallikrein 12, kallikrein 14, and kallikrein 15 levels in a patient and the normal levels is an indication that the patient is afflicted with or has a predisposition to endocrine cancer.

In an embodiment, the method detects kallikrein 14 and the amount of kallikrein 14 detected is greater than that of a standard and is indicative of endocrine cancer, more particularly ovarian or breast cancer.

According to a method involving kallikrein 14, the level of kallikrein 14 in a sample is compared with the normal levels of kallikrein 14 in samples of the same type obtained from controls (e.g. samples from individuals not afflicted with cancer). Significantly altered levels in the sample of the kallikrein 14 (e.g. higher levels) relative to the normal levels in a control is indicative of cancer.

Kallikrein 12, kallikrein 14, and/or kallikrein 15 may be measured using a reagent that detects or binds to kallikrein 12, kallikrein 14, and/or kallikrein 15, in particular binding agents, more particularly, antibodies specifically reactive with kallikrein 12, kallikrein 14, and/or kallikrein 15 or a part thereof.

In an embodiment, the invention provides methods for determining the presence or absence of endocrine cancer in a patient, comprising the steps of (a) contacting a biological sample obtained from a patient with a binding agent that specifically binds to kallikrein 12, kallikrein 14, and/or kallikrein 15 protein; and (b) detecting in the sample an amount of protein that binds to the binding agent, relative to a predetermined standard or cut-off value, and therefrom determining the presence or absence of endocrine cancer in the patient.

In another embodiment, the invention relates to a method for diagnosing and monitoring endocrine cancer in a subject by quantitating kallikrein 12, kallikrein 14, and/or kallikrein 15 in a biological sample from the subject comprising (a) reacting the biological sample with a binding agent specific for kallikrein 12, kallikrein 14, and/or kallikrein 15 (e.g. an antibody) which is directly or indirectly labelled with a detectable substance; and (b) detecting the detectable substance.

In another aspect the invention provides a method for using an antibody to detect expression of a kallikrein 12, kallikrein 14, and/or kallikrein 15 protein in a sample, the method comprising: (a) combining an antibody specific for kallikrein 12, kallikrein 14, and/or kallikrein 15 with a sample under conditions which allow the formation of antibody-protein complexes; and (b) detecting complex formation, wherein complex formation indicates expression of the protein in the sample. Expression may be compared with standards and is diagnostic of endocrine cancer.

Embodiments of the methods of the invention involve (a) reacting a biological sample from a subject with an antibody specific for kallikrein 12, kallikrein 14, and/or kallikrein 15 which is directly or indirectly labelled with an enzyme; (b) adding a substrate for the enzyme wherein the substrate is selected so that the substrate, or a reaction product of the enzyme and substrate forms fluorescent complexes; (c) quantitating kallikrein 12, kallikrein 14, and/or kallikrein 15 in the sample by measuring fluorescence of the fluorescent complexes; and (d) comparing the quantitated levels to levels obtained for other samples from the subject patient, or control subjects.

In another embodiment the quantitated levels are compared to levels quantitated for control subjects (normal or benign) without endocrine cancer wherein an increase in kallikrein 14 levels compared with the control subjects is indicative of endocrine cancer, in particular ovarian or breast cancer.

A particular embodiment of the invention comprises the following steps
(a) incubating a biological sample with a first antibody specific for kallikrein 12, kallikrein 14, and/or kallikrein 15 which is directly or indirectly labeled with a detectable substance, and a second antibody specific for kallikrein 12, kallikrein 14, and/or kallikrein 15 which is immobilized;
(b) detecting the detectable substance thereby quantitating kallikrein 12, kallikrein 14, and/or kallikrein 15 in the biological sample; and
(c) comparing the quantitated kallikrein 12, kallikrein 14, and/or kallikrein 15 with levels for a predetermined standard.

The standard may correspond to levels quantitated for samples from control subjects without endocrine cancer (normal or benign), with a different disease stage, or from other samples of the subject Increased levels of kallikrein 14 as compared to the standard is indicative of endocrine cancer.

Other methods of the invention employ one or more polynucleotides, oligonucleotides, or nucleic acids capable of hybridizing to polynucleotides encoding kallikrein 12, kallikrein 14, and/or kallikrein 15. Thus, methods for detecting KLK12, KLK14, and/or KLK15 can be used to monitor endocrine cancer by detecting KLK12, KLK14, and/or KLK15 nucleic acids. In an aspect, KLK14, KLK15, or KLK14 and KLK15 are detected.

Thus, the present invention relates to a method for diagnosing and monitoring endocrine cancer in a sample from a subject comprising isolating nucleic acids, preferably mRNA, from the sample; and detecting KLK12, KLK14, and/or KLK15 nucleic acids in the sample. The presence of different levels of KLK12, KLK14, and/or KLK15 nucleic acids in the sample compared to a standard or control may be indicative of disease, disease stage, and/or a positive prognosis i.e. longer progression-free and overall survival.

In an embodiment of the invention, KLK-14-positive tumors (e.g. higher levels of KLK-14 compared to a control from cancerous tissue or advanced stage cancer) are a positive diagnostic indicator. KLK-14-positive tumors may be indicative of early stage cancer, in particular early stage ovarian cancer, optimal debulking, responsiveness to chemotherapy, longer progression-free survival and/or overall survival.

In an embodiment of the invention, KLK-15-positive tumors (e.g. higher levels of KLK-15 compared to a control normal or benign tissue) are a negative diagnostic indicator. KLK-15-positive tumors may be indicative of endocrine cancer, in particular ovarian cancer, advanced stage disease, lower progression-free survival, and/or overall survival.

The invention provides methods for determining the presence or absence of endocrine cancer in a subject comprising detecting in the sample levels of polynucleotides that hybridize to nucleic acids encoding kallikrein 12, kallikrein 14, and/or kallikrein 15, comparing the levels with a predetermined standard or cut-off value, and therefrom determining the presence or absence of endocrine cancer in the subject In an embodiment, the invention provides methods for determining the presence or absence of endocrine cancer in a subject comprising (a) contacting a sample obtained from the subject with an oligonucleotide that hybridizes to a nucleic acid molecule encoding kallikrein 12, kallikrein 14, and/or kallikrein 15; and (b) detecting in the sample a level of polynucleotide that hybridizes to the nucleic acid molecule relative to a predetermined cut-off value, and therefrom determining the presence or absence of endocrine cancer in the subject.

Within certain embodiments, the amount of polynucleotide that is mRNA is detected via polymerase chain reaction using, for example, at least one oligonucleotide primer that hybridizes to a nucleic acid molecule that encodes kallikrein 12, kallikrein 14, and/or kallikrein 15, or a complement of such nucleic acid molecule. Within other embodiments, the amount of mRNA is detected using a hybridization technique, employing an oligonucleotide probe that hybridizes to kallikrein 12, kallikrein 14, and/or kallikrein 15, or a complement of such nucleic acid molecule.

When using mRNA detection, the method may be carried out by combining isolated mRNA with reagents to convert to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of KLK12, KLK14, and/or KLK15 marker in the sample. For mRNA the analyzing step may be accomplished using Northern Blot analysis to detect the presence of endocrine KLK12, KLK14, and/or KLK15 marker. The analysis step may be further accomplished by quantitatively detecting the presence of endocrine KLK12, KLK14, and/or KLK15 marker in the amplification product, and comparing the quantity of marker detected against a panel of expected values for the known presence or absence of the marker in normal and malignant tissue derived using similar primers.

Therefore, the invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to a nucleic acid molecule encoding kallikrein 12, kallikrein 14 and/or kallikrein 15, to produce amplification products; (d) analyzing the amplification products to detect an amount of mRNA encoding kallikrein 12, kallikrein 14 and/or kallikrein 15; and (e) comparing the amount of mRNA to an amount detected against a panel of expected values for normal and malignant tissue derived using similar nucleic acid primers.

The invention also contemplates a method comprising administering to cells or tissues imaging agents that carry labels for imaging and bind to kallikrein 12, kallikrein 14, and/or kallikrein 15 and optionally other markers of endocrine cancer, and then imaging the cells or tissues.

In an aspect the invention provides an in vivo method comprising administering to a subject an agent that has been constructed to target one or more kallikreins, in particular kallikrein 12, kallikrein 14, and/or kallikrein 15.

The invention therefore contemplates an in vivo method comprising administering to a mammal one or more agent that carries a label for imaging and binds to a kallikrein, preferably kallikrein 12, kallikrein 14, and/or kallikrein 15, and then imaging the mammal.

According to a particular aspect of the invention, an in vivo method for imaging endocrine cancer is provided comprising:
(a) injecting a patient with agents that bind to kallikrein 12, kallikrein 14, and/or kallikrein 15, the agents carrying labels for imaging the endocrine cancer,
(b) allowing the agents to incubate in vivo and bind to kallikrein 12, kallikrein 14, and/or kallikrein 15 associated with the endocrine cancer, and
(c) detecting the presence of the labels localized to the endocrine cancer.

In an embodiment of the invention an agent is an antibody which recognizes the kallikrein. In another embodiment of the invention the agent is a chemical entity which recognizes the kallikrein.

The agent carries a label to image the kallikreins and optionally other markers. Examples of labels useful for imaging are radiolabels, fluorescent labels (e.g fluorescein and rhodamine), nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed.

The invention also contemplates the localization or imaging methods described herein using multiple markers for endocrine cancer.

The invention also relates to kits for carrying out the methods of the invention. In an embodiment, the kit is for assessing whether a patient is afflicted with endocrine cancer, in particular ovarian or breast cancer.

The invention also provides a diagnostic composition comprising a kallikrein 12, kallikrein 14, and/or kallikrein 15 protein or nucleic acids encoding kallikrein 12, kallikrein 14, and/or kallikrein 15. In an embodiment, the composition comprises probes that specifically hybridize to KLK12, KLK14, or KLK15 or fragments thereof, or antibodies specific for kallikrein 12, kallikrein 14, and/or kallikrein 15 or fragments thereof. In another embodiment a composition is provided comprising KLK12, KLK14, or KLK15 specific primer pairs capable of amplifying KLK12, KLK14, or KLK15 using polymerase chain reaction methodologies. The probes, primers or antibodies can be labeled with a detectable substance.

Still further the invention relates to therapeutic applications for endocrine cancer employing kallikrein 12, kallikrein 14, and/or kallikrein 15 proteins, nucleic acid molecules encoding the proteins, and/or binding agents for the proteins.

In an aspect, the invention relates to compositions comprising kallikrein 12, kallikrein 14, and/or kallikrein 15 or part thereof, or an antibody specific for kallikrein 12, kallikrein 14, and/or kallikrein 15, and a pharmaceutically acceptable carrier, excipient, or diluent A method for treating or preventing endocrine cancer in a patient is also provided comprising administering to a patient in need thereof, kallikrein 12, kallikrein 14, and/or kallikrein 15 or part thereof, an antibody specific for kallikrein 12, kallikrein 14, and/or kallikrein 15, or a composition of the invention. In an aspect the invention provides a method of treating a patient afflicted with or at risk of developing endocrine cancer comprising inhibiting expression of kallikrein 12, kallikrein 14, and/or kallikrein 15.

In an aspect, the invention provides antibodies specific for kallikrein 12, kallikrein 14, and/or kallikrein 15 that can be used therapeutically to destroy or inhibit the growth of kallikrein 12, kallikrein 14, and/or kallikrein 15 expressing endocrine cancer cells, (e.g. ovarian cancer or breast cancer cells), or to block kallikrein 12, kallikrein 14, and/or kallikrein 15 activity. In addition, kallikrein 12, kallikrein 14, and/or kallikrein 15 may be used in various immunotherapeutic methods to promote immune-mediated destruction or growth inhibition of tumors expressing kallikrein 12, kallikrein 14, and/or kallikrein 15.

The invention also contemplates a method of using kallikrein 12, kallikrein 14, and/or kallikrein 15 or part thereof, or an antibody specific for kallikrein 12, kallikrein 14, and/or kallikrein 15 in the preparation or manufacture of a medicament for the prevention or treatment of endocrine cancer.

Another aspect of the invention is the use of kallikrein 12, kallikrein 14, and/or kallikrein 15, peptides derived therefrom, or chemically produced (synthetic) peptides, or any combination of these molecules, for use in the preparation of vaccines to prevent endocrine cancer and/or to treat endocrine cancer.

The invention contemplates vaccines for stimulating or enhancing in a subject to whom the vaccine is administered production of antibodies directed against kallikrein 12, kallikrein 14, and/or kallikrein 15, in particular kallikrein 14.

The invention also provides a method for stimulating or enhancing in a subject production of antibodies directed against kallikrein 12, kallikrein 14, and/or kallikrein 15, in particular kallikrein 14. The method comprises administering to the subject a vaccine of the invention in a dose effective for stimulating or enhancing production of the antibodies.

The invention further provides a method for treating, preventing, or delaying recurrence of endocrine cancer. The methods comprise administering to the subject a vaccine of the invention in a dose effective for treating, preventing, or delaying recurrence of endocrine cancer.

In particular embodiments of the invention the methods, compositions and kits use KLK14 and KLK15, and/or kallikrein 14.

The invention also contemplates the methods, compositions, and kits described herein using additional markers associated with endocrine cancer. The methods described herein may be modified by including reagents to detect the additional markers, or nucleic acids for the markers. Other markers include human stratum corneum chymotryptic enzyme (HSCCE), haptoglobin alpha, osteopontin, inhibin, prostasin, lipid-associated sialic acid, (LASA), kallikrein 2, kallikrein 3, kallikrein 4, kallikrein 5, kallikrein 6, kallikrein 8, kallikrein 9, kallikrein 10, kallikrein 11, and kallikrein 13; CA125, CA15.3, CA72-4, CA 19-9, OVX1, lysophosphatidic acid (LPA), creatin-kinase BB, and carcinoembryonic antigen (CEA). In particular, the other markers are markers to kallikreins. In an aspect of the invention, the markers are one or more of kallikrein 5, kallikrein 6, kallikrein 8, kallikrein 10, kallikrein 11, and kallikrein 13 or nucleic acids encoding kallikrein 4, kallikrein 5, kallikrein 6, kallikrein 7, kallikrein 8, kallikrein 9 and kallikrein 10. The methods described herein may be modified by including reagents to detect the markers, or nucleic acids for the markers.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which

FIG. 1 shows the quantification of KLK14 gene expression by real-time PCR. (A): A logarithmic plot of fluorescence signal versus cycle number. Serial dilutions of a total RNA preparation from ovarian tissue were made (10-fold at a time) and an arbitrary copy number was assigned to each sample, according to the dilution factor. Each sample was analyzed in duplicate. (B): A representative calibration curve for KLK14 mRNA quantification.

FIG. 2 shows the KLK14 mRNA concentration in four breast cancer cell lines, 24 hours after stimulation with steroids at a concentration of 10⁻⁸ M. DHT, dihydrotestosterone.

FIG. 3 shows KLK14 mRNA concentration in two ovarian cancer cell lines, 24 hours after stimulation with steroids at a concentration of 10⁻⁸ DHT, dihydrotestosterone.

FIG. 4 shows KLK14 mRNA concentration in the BT-474 breast cancer cell line before (0) and at 2, 6, 12 and 24 hours after stimulation with dihydrotestosterone at a concentration of 10⁻⁸ M.

FIG. 5 shows KLK14 mRNA concentration in the BT-474 cell line 24 hours after dihydrostestosterone (DHT) stimulation with and without blocking by Nilutamide. Nilutamide was added at a concentration of 10⁻⁶ M and DHT was added 1 h later, at a concentration of 10⁻⁸ M. Alcohol was added as control.

FIG. 6 shows the distribution of KLK14 mRNA concentration in normal ovarian tisssues, benign ovarian tumors and ovarian cancer tissues. The horizontal lines represent median values. p was calculated by Kruskal-Wallis test. N=number of samples. p value was calculated by Kruskal-Wallis Test N=number of samples.

FIG. 7 shows Kaplan-Meier survival curves for the progression-free survival (PFS) (A) and overall survival (B) in patients with KLK14-positive and negative ovarian tumors. n=number of samples.

FIG. 8 shows a correlation between serum CA125 and tumor levels of KLK14 mRNA. rₛ=Spearman correlation coefficient N=number of patients.

FIG. 9 shows the quantification of KLK15 gene expression by real-time PCR (A) A logarithmic plot of fluorescence signal versus cycle number. (B) A representative calibration curve for KLK15 mRNA quantification. Curves were obtained with serially diluted (10-fold) plasmid containing KLK15 cDNA.

FIG. 10 shows the distribution of KLK15 expression levels in cancerous and benign ovarian tissues. Horizontal lines show median values (p=0.021 by Mann-Whitney U test).

FIG. 11 shows Kaplan-Meier survival curves for the progression-free survival (PFS) (A) and overall survival (B) in patients with KLK15-positive and negative ovarian tumors. n=number of samples.

FIG. 12 shows a correlation between serum CA125 and tumor levels of KLK15 expression. rₛ=Spearman correlation coefficient.

FIG. 13 shows expression and purification of recombinant kallikrein 14^{myc-his}. A) Detection of recombinant hK14^{myc-His} by western blot analysis using anti-His antibody. Lane 1, molecular weight markers; Lane 2, culture supernatant from a yeast clone transformed with pPICZαA vector containing KLK14 cDNA prior to methanol induction (Day 0); Lanes 3,4 and 5 correspond to 2, 4 and 6 days of induction with 1% methanol. Lane 6, supernatant from an X-33 strain transformed with empty pPICZαA (negative control) after 6 days of methanol induction (1%). B) The proteins were separated by SDS-PAGE and stained with Coomassie blue. Lane 1, Recombinant hK14^{myc-His,} purified by IMAC from the yeast culture supernatant. Elution fractions were concentrated 20 times (1.5 mg/mL as determined by the BCA total protein assay). M=molecular weight marker

FIG. 14 show an SDS-PAGE of purified hK14^{myc-his} before and after treatment with PNGase-F. In both A) and B) lane 1 corresponds to purified hK14^{myc-His}; lane 2, purified hK14^{myc-His} incubated with PNGase-F (38 kDa); lane 3, horseradish peroxidase (40 kDa), positive control; and lane 4, soybean trypsin inhibitor (21.5 kDa), negative control. (Note that in lane 2 of A the upper band represents PNGase-F). In A), the gel was stained with Coomassie-blue and there is no noticeable shift in the band representing hK14^{myc-His} in lane 2, as would be expected following deglycosylation. In B), the gel was stained with acidic acidic fuchsin sulfite, a glycoprotein stain. Only the glycoprotein horseradish peroxidase (positive control) in lane 3 is stained, further confirming that recombinant hK14^{myc-His} is not glycosylated. M=molecular weight marker

FIG. 15 shows a typical calibration curve for hK14 ELISA. Background fluorescence (zero calibrator) has been subtracted from all measurements. The dynamic range of this assay is 0.1-20 μg/L.

FIG. 16 is a bar diagram showing the specificity of the hK14 immunoassay. The immunoreactivity of pre-immune and immune mouse and rabbit sera was assessed by performing the immunoassay with mouse and rabbit immune sera (white bars), replacing mouse serum with pre-immune mouse serum (striped bars) and replacing rabbit serum with pre-immune rabbit serum (grey bars). Note the significant decrease in fluorescence (immunoreactivity) when immune hK14-sera are replaced with pre-immune sera.

FIG. 17 shows the specificity of rabbit anti-hK14^{myc-his} polyclonal antibodies. Purified recombinant hK14^{myc-His} (1000, 100 and 20 ng/lane, in lanes 1, 2, 3, respectively) was separated by SDS-PAGE, transferred to a nitrocellulose membrane and probed with pre-immune (A) and immune (B) rabbit sera (1:2000). Note the absence of bands in (A) indicating the absence of hK14-specific antibodies, as anticipated. In (B), hK14 specific bands are visualized. Band intensity diminishes with decreasing amounts of hK14^{myc-His}. M=molecular weight marker

FIG. 18 shows the concentration of hK14 in various adult human tissues.

FIG. 19 shows the immunohistochemical localization of hK14 in normal (A and B) and malignant (C and D) tissues with the polyclonal hK14 rabbit antibody. A) Cytoplasmic immunoexpression in ductal columnar and myoepithelial cells of the breast (magnification ×200). B) Strong immunoexpression by the epithelium of the eccrine glands of the skin (magnification ×200). C) Staining by a moderately differentiated cystadenocarcinoma of the ovary (magnification ×200). D) Strong cytoplasmic immunoexpression by epithelium with apocrine metaplasia of the breast (magnification ×100).

FIG. 20 shows the hormonal regulation of hK14 in the breast cancer cell line BT-474. hK14 is mainly up-regulated by estradiol, followed by dihydrotestosterone (DHT) and norgestrel.

FIG. 21 show the concentration of hK14 in extracts from normal, benign and cancerous ovarian tissues. N=number of tissues extracted. The percentage of samples containing higher hK14 levels compared to highest normal tissue extract is shown.

FIG. 22 shows serum hK14 levels in patients with ovarian and breast cancer compared to normal females. Serum hK14 is elevated in 65% of women with ovarian cancer and 40% with breast cancer when using a cutoff value equal to the lower detection limit (0.1 μg/L) of the immunoassay (indicated by the dotted line).

FIG. 23 is a schematic diagram of the configuration of a hK14 immunoassay. Microtitre well plates were coated with sheep anti-mouse IgG, and subsequently incubated with mouse anti-hK14 polyclonal antibodies, hK14 standards/samples, rabbit anti-hK14 polyclonal antibodies, ALP (alkaline phosphatase) conjugated goat anti-rabbit IgG and DFP (diflunisal phosphate). After removal of a phosphate ester group from DFP by ALP, it forms a complex with Tb³⁺-EDTA (a lanthanide chelate). After excitation of the ligand at 336 nm, the fluorescence of Tb³⁺ (624 nm) is measured by time-resolved fluorometry on the Cyberfluor 615 gated fluorometer. Fluorescence measurements are taken throughout the first 100-200 μs after excitation, eliminating all short-lived fluorescence background signals due to solvents, cuvettes, and reagents, as well as scattered excitation radiation. The fluorescence intensity is proportional to the amount of hK14 present in standards or samples

FIG. 24 shows a typical calibration curve for the hK14 immunoassay. The background fluorescence (zero calibrator) has been subtracted from all measurements. The dynamic range of this assay is 0.1-20 μg/L.

FIG. 25 is a graph showing the linearity of the hK14 immunoassay. This figure shows the concentration of hK14 in a serially diluted seminal plasma sample.

FIG. 26 are graphs showing the correlation between hK14 and hK3 (A), hK6 (B) and hK10 (C) concentration in breast tumors. The strength of associations between the above variables was tested with the Spearman rank correlation (rₛ).

### DETAILED DESCRIPTION OF THE INVENTION

The invention relates to newly discovered correlations between expression of kallikrein 12, kallikrein 14, and/or kallikrein 15 and nucleic acids encoding kallikrein 12, kallikrein 14, and/or kallikrein 15 and cancer, in particular ovarian, breast, or prostate cancer. The markers described herein provide sensitive and/or specific methods for detecting cancer.

Methods are provided for detecting the presence of cancer in a sample, the absence of cancer in a sample, the stage of a cancer, the grade of the cancer, the benign or malignant nature of a cancer, the metastatic potential of a cancer, assessing the histological type of neoplasm associated with the cancer, the indolence or aggressiveness of the cancer, and other characteristics of cancer that are relevant to prevention, diagnosis, characterization, and therapy of cancer in a patient Methods are also provided for assessing the efficacy of one or more test agents for inhibiting cancer, assessing the efficacy of a therapy for cancer, monitoring the progression of cancer, selecting an agent or therapy for inhibiting cancer, treating a patient afflicted with cancer, inhibiting cancer in a patient, and assessing the carcinogenic potential of a test compound.

Glossary

The terms “sample”, “biological sample”, and the like mean a material known or suspected of expressing or containing KLK12, KLK14, and/or KLK15 or kallikrein 12, kallikrein 14, and/or kallikrein 15, in particular KLK12, KLK14, and/or KLK15 or kallikrein 12, kallikrein 14, and/or kallikrein 15 associated with endocrine cancer. The test sample can be used directly as obtained from the source or following a pretreatment to modify the character of the sample. The sample can be derived from any biological source, such as tissues, extracts, or cell cultures, including cells (e.g. tumor cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid and the like. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Nucleic acids and proteins may be isolated from the samples and utilized in the methods of the invention.

In embodiments of the invention the sample is a mammalian tissue sample. In another embodiment the sample is a human physiological fluid. In a particular embodiment, the sample is human serum.

The terms “subject”, “individual” or “patient” refer to a warm-blooded animal such as a mammal, which is afflicted with or suspected of having or being pre-disposed to endocrine cancer or condition as described herein. In particular, the terms refer to a human.

The term “kallikrein 12”, “kallikrein 12 polypeptide” or “kallikrein 12 protein” includes human kallikrein 12 (“hK12”), in particular the native-sequence polypeptide, isoforms, chimeric polypeptides, all homologs, fragments, and precursors of human kallikrein 21. The amino acid sequence for native hK12 include the sequences of GenBank Accession No. AAF06065, AAD26426, and AAF06066 and shown in SEQ ID NO. 1, 2 and 3.

The term “kallikrein 14”, “kallikrein 14 polypeptide” or “kallikrein 14 protein” includes human kallikrein 14 (“hK14”), in particular the native-sequence polypeptide, isoforms, chimeric polypeptides, all homologs, fragments, and precursors of human kallikrein 14. The amino acid sequence for native hK14 include the sequences of GenBank Accession No. AAK48524 and AAD50773 and shown in SEQ ID NO. 5.

The term “kallikrein 15”, “kallikrein 15 polypeptide” or “kallikrein 15 protein” includes human kallikrein 15 (“hK15”), in particular the native-sequence polypeptide, isoforms, chimeric polypeptides, all homologs, fragments, and precursors of human kallikrein 15. The amino acid sequence for native hK15 include the sequences of GenBank Accession No. AAG09469, AAG09472, AAG09471, and AAG09470 and shown in SEQ ID NO. 7, 8, 9, and 10.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g alternatively spliced forms or splice variants), and naturally occurring allelic variants.

The term “polypeptide variant” means a polypeptide having at least about 70-80%, preferably at least about 85%, more preferably at least about 90%, most preferably at least about 95% amino acid sequence identity with a native-sequence polypeptide, in particular having at least 70-80%, 85%, 90%, 95% amino acid sequence identity to the sequences identified in the GenBank Accession Nos. AAF06065, AAD26426, AAF06066, AAK48524, AAD50773, AAG09469, AAG09472, AAG09471, and AAG09470 and shown in SEQ ID NOs. 1, 2, 3, 5, 7, 8, 9, and 10. Such variants include, for instance, polypeptides wherein one or more amino acid residues are added to, or deleted from, the N— or C-terminus of the full-length or mature sequences of SEQ ID NO: 1, 2, 3, 5, 7, 8, 9, and 10, including variants from other species, but excludes a native-sequence polypeptide.

An allelic variant may also be created by introducing substitutions, additions, or deletions into a nucleic acid encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a kallikrein polypeptide homolog, for example, the murine kallikrein polypeptide.

The invention also includes polypeptides that are substantially identical to the sequences of GenBank Accession Nos. AAF06065, AAD26426, AAF06066, AAK48524, AAD50773, AAG09469, AAG09472, AAG09471, and AAG09470 and shown in SEQ ID NOs. 1, 2, 3, 5, 7, 8, 9, and 10. (e.g. at least about 45%, preferably 50%/0, 55%, 60%, 65%, 70%, 750/o, 80%, 85%, 90%, 95%, 97%, 98%, or 99% sequence identity), and in particular polypeptides that retain the immunogenic activity of the corresponding native-sequence polypeptide.

Percent identity of two amino acid sequences, or of two nucleic acid sequences identified herein is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NMH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

Kallikrein 12, kallikrein 14, and kallikrein 15 proteins include chimeric or fusion proteins. A “chimeric protein” or “fusion protein” comprises all or part (preferably biologically active) of a kallikrein 12, kallikrein 14, or kallikrein 15 polypeptide operably linked to a heterologous polypeptide (i.e., a polypeptide other than a kallikrein 12, kallikrein 14, or kallikrein 15 polypeptide). Within the fusion protein, the term “operably linked” is intended to indicate that a kallikrein 12, kallikrein 14, or kallikrein 15 polypeptide and the heterologous polypeptide are fused in-frame to each other. The heterologous polypeptide can be fused to the N-terminus or C-terminus of a kallikrein 12, kallikrein 14, or kallikrein 15 polypeptide. A useful fusion protein is a GST fusion protein in which a kallikrein 12, kallikrein 14, or kallikrein 15 polypeptide is fused to the C-terminus of GST sequences. Another example of a fusion protein is an immunoglobulin fusion protein in which all or part of a kallikrein 12, kallikrein 14, or kallikrein 15 polypeptide is fused to sequences derived from a member of the immunoglobulin protein family. Chimeric and fusion proteins can be produced by standard recombinant DNA techniques.

Kallikrein polypeptides may be isolated from a variety of sources, such as from human tissue types or from another source, or prepared by recombinant or synthetic methods, or by any combination of these and similar techniques.

“KLK12”, “KLK12 polynucleotide(s)” or “KLK12 nucleic acid molecules” refers to polynucleotides encoding kallikrein 12 including a native-sequence polypeptide, a polypeptide variant including a portion of a kallikrein 12 polypeptide, an isoform, precursor, and a chimeric polypeptide. A polynucleotide encoding a native kallikrein 12 polypeptide employed in the present invention includes the nucleic acid sequence of GenBank Accession No. AF135025 (SEQ ID NO 4), or a fragment thereof.

“KLK14”, “KLK14 polynucleotide(s)” or “KLK14 nucleic acid molecules” refers to polynucleotides encoding kallikrein 14 including a native-sequence polypeptide, a polypeptide variant including a portion of a kallikrein 14 polypeptide, an isoform, precursor, and a chimeric polypeptide. A polynucleotide encoding a native kallikrein 14 polypeptide employed in the present invention includes the nucleic acid sequences of GenBank Accession Nos. AF161221 and AF283670 (SEQ ID NO 6 and 21), or a fragment thereof.

“KLK15”, “KLK15 polynucleotide(s)” or “KLK14 nucleic acid molecules” refers to polynucleotides encoding kallikrein 15 including a native-sequence polypeptide, a polypeptide variant including a portion of a kallikrein 15 polypeptide, an isoform, precursor, and a chimeric polypeptide. A polynucleotide encoding a native kallikrein 15 polypeptide employed in the present invention includes the nucleic acid sequence of GenBank Accession No. No. AF242195 (SEQ ID NO 11), or a fragment thereof

KLK12, KLK14, KLK15 polynucleotides include complementary nucleic acid sequences, and nucleic acids that are substantially identical to these sequences (e.g. at least about 45%, preferably 50%, 55%, 60%, 65%, 70%, 75%, 80%, 85%, 90%, 95%, 97%, 98%, or 99% sequence identity).

KLK12, KLK14, and KLK15 polynucleotides also include sequences that differ from a nucleic acid sequence of GenBank Accession No. AF135025, AF161221, AF283670, and AF242195 (SEQ ID NOs: 4,6, 21, and 11) due to degeneracy in the genetic code. As one example, DNA sequence polymorphisms within the nucleotide sequence of a kallikrein 12, kallikrein 14, or kallikrein 15 polypeptide may result in silent mutations that do not affect the amino acid sequence. Variations in one or more nucleotides may exist among individuals within a population due to natural allelic variation. DNA sequence polymorphisms may also occur which lead to changes in the amino acid sequence of a kallikrein polypeptide.

KLK12, KLK14, and KLK15 polynucleotides also include nucleic acids that hybridize under stringent conditions, preferably high stringency conditions to a nucleic acid sequence of GenBank Accession Nos. AF135025, AF161221, AF283670, and AF242195 (SEQ ID NOs: 4, 6, 21, and 1). Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C, followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

KLK12, KLK14, and KLK15 polynucleotides also include truncated nucleic acids or nucleic acid fragments and variant forms of the nucleic acids that arise by alternative splicing of an mRNA corresponding to a DNA.

The KLK12, KLK14, and KLK15 polynucleotides are intended to include DNA and RNA (e.g. mRNA) and can be either double stranded or single stranded. A nucleic acid may, but need not, include additional coding or non-coding sequences, or it may, but need not, be linked to other molecules and/or carrier or support materials. The nucleic acids for use in the methods of the invention may be of any length suitable for a particular method. In certain applications the term refers to antisense nucleic acid molecules (e.g. an mRNA or DNA strand in the reverse orientation to a sense KLK12, KLK14, and KLK15).

“Statistically different levels”, “significantly altered levels”, or “significant difference” in levels of markers in a patient or subject sample compared to a control or standard (e.g. normal levels or levels in other samples from a patient) may represent levels that are higher or lower, in particular lower, than the standard error of the detection assay. In particular embodiments, the levels may be 1.5, 2, 3, 4, 5, or 6 times higher or lower than the control or standard.

“Binding agent” refers to a substance such as a polypeptide or antibody that specifically binds to a kallikrein 12, kallikrein 14, and/or kallikrein 15 protein. A substance “specifically binds” to a kallikrein 12, kallikrein 14, and/or kallikrein 15 protein if is reacts at a detectable level with kallikrein 12, kallikrein 14, and/or kallikrein 15, and does not react detectably with peptide containing an unrelated sequence or a sequence of a different kallikrein. Binding properties may be assessed using an ELISA, which may be readily performed by those skilled in the art (see for example, Newton et al, Develop. Dynamics 197: 1-13, 1993).

A binding agent may be a ribosome, with or without a peptide component, an RNA molecule, or a polypeptide. A binding agent may be a polypeptide that comprises an kallikrein 12, kallikrein 14, and/or kallikrein 15 sequence, a peptide variant thereof, or a non-peptide mimetic of such a sequence. By way of example a kallikrein 12 sequence may be a peptide portion of a kallikrein 12 that is capable of modulating a function mediated by kallikrein 12. Similarly a kallikrein 14 sequence may be a peptide portion of a kallikrein 14 that is capable of modulating a function mediated by kallikrein 14, and a kallikrein 15 sequence may be a peptide portion of a kallikrein 15 that is capable of modulating a function mediated by kallikrein 15.

Antibodies for use in the present invention include but are not limited to monoclonal or polyclonal antibodies, immunologically active fragments (e.g. a Fab or (Fab)₂ fragments), antibody heavy chains, humanized antibodies, antibody light chains, genetically engineered single chain Fᵥ molecules (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native or recombinant kallikrein 12, kallikrein 14, or kallikrein 15 may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030, and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for kallikrein 12, kallikrein 14, or kallikrein 15 may also be obtained from scientific or commercial sources.

In an embodiment of the invention, antibodies are reactive against kallikrein 12, kallikrein 14, or kallikrein 15 if they bind with a K. of greater than or equal to 10⁻⁷ M.

“Endocrine cancer” or “endocrine carcinoma” includes but is not limited to cancers of reproductive organs such as ovarian cancer, breast cancer, and prostate cancer. Particular embodiments based on KLK14 and KLK15 are useful for ovarian cancer applications. Particular embodiments based on kallikrein 14 are useful for ovarian or breast cancer applications.

Methods

A variety of methods can be employed for the diagnostic and prognostic evaluation of endocrine cancer involving kallikrein 12, kallikrein 14, and/or kallikrein 15 proteins and nucleic acids encoding the proteins, and the identification of subjects with a predisposition to such disorders. Such methods may, for example, utilize KLK12, KLK14, and/or KLK15 nucleic acids, and fragments thereof, and binding agents (e.g. antibodies) against kallikrein 12, kallikrein 14, and/or kallikrein 15, including peptide fragments. In particular, the nucleic acids and antibodies may be used, for example, for (1) the detection of the presence of KLK12, KLK14, and/or KLK15 mutations, or the detection of either over- or under-expression of KLK12, KLK14, and/or KLK15 mRNA relative to a non-disorder state or the qualitative or quantitative detection of alternatively spliced forms of KLK12, KLK14, and/or KLK15 transcripts which may correlate with certain conditions or susceptibility toward such conditions; and (2) the detection of either an over- or an under-abundance of kallikrein 12, kallikrein 14, and/or kallikrein 15 relative to a non-disorder state or the presence of a modified (e.g., less than full length) kallikrein 12, kallikrein 14, and/or kallikrein 15 which correlates with a disorder state, or a progression toward a disorder state.

The invention also contemplates a method for detecting endocrine cancer comprising producing a profile of levels of kallikrein 12, kallikrein 14, and/or kallikrein 15 proteins and/or nucleic acids encoding the proteins, and other markers associated with endocrine cancer in cells from a patient, and comparing the profile with a reference to identify a profile for the test cells indicative of disease.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of disease. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein can be adapted for diagnosing and monitoring endocrine carcinoma by detecting kallikrein 12, kallikrein 14, and/or kallikrein 15 protein or nucleic acid molecules encoding the protein in biological samples from a subject. These applications require that the amount of protein or nucleic acid molecule quantitated in a sample from a subject being tested be compared to a predetermined standard or cut-off value. The standard may correspond to levels quantitated for another sample or an earlier sample from the subject, or levels quantitated for a control sample. Levels for control samples from healthy subjects or endocrine cancer subjects may be established by prospective and/or retrospective statistical studies. Healthy subjects who have no clinically evident disease or abnormalities may be selected for statistical studies. Diagnosis may be made by a finding of statistically different levels of detected kallikrein 12, kallikrein 14, and/or kallikrein 15 or KLK12, KLK14, and/or KLK15, compared to a control sample or previous levels quantitated for the same subject.

The methods described herein may also use multiple markers for endocrine cancer. Therefore, the invention contemplates a method for anaylzing a biological sample for the presence of kallikrein 12, kallikrein 14, and/or kallikrein 15 proteins and nucleic acids encoding the kallikreins, and other markers that are specific indicators of endocrine cancer. Other markers include human stratum corneum chymotryptic enzyme (HSCCE), haptoglobin alpha, osteopontin, inhibin, prostasin, lipid-associated sialic acid, (LASA), kallikrein 2, kallikrein 3, kallikrein 4, kallikrein 5, kallikrein 6, kallikrein 8, kallikrein 9, kallikrein 10, kallikrein 11, and kallikrein 13; CA125, CA15.3, CA72-4, CA19-9, OVX1, lysophosphatidic acid (LPA), creatin-kinase BB, and carcinoembryonic antigen (CEA). In particular, the other markers are markers to kallikreins. In an aspect of the invention, the markers are one or more of kallikrein 5, kallikrein 6, kallikrein 8, kallikrein 10, kallikrein 11, and kallikrein 13 or nucleic acids encoding kallikreins 4 to 10. The methods described herein may be modified by including reagents to detect the markers, or nucleic acids for the markers.

Nucleic Acid Methods/Assays

As noted herein an endocrine cancer may be detected based on the level of KLK12, KLK14, and/or KLK15 in a sample. Techniques for detecting nucleic acid molecules such as polymerase chain reaction (PCR) and hybridization assays are well known in the art.

Probes may be used in hybridization techniques to detect KLK12, KLK14, and/or KLK15 nucleic acids. The technique generally involves contacting and incubating nucleic acids (e.g. recombinant DNA molecules, cloned genes) obtained from a sample from a patient or other cellular source with a probe under conditions favorable for the specific annealing of the probes to complementary sequences in the nucleic acids. After incubation, the non-annealed nucleic acids are removed, and the presence of nucleic acids that have hybridized to the probe if any are detected.

Nucleotide probes for use in the detection of nucleic acid sequences in samples may be constructed using conventional methods known in the art. Suitable probes may be based on nucleic acid sequences encoding at least 5 sequential amino acids from regions of KLK12, KLK14, and/or KLK15 nucleic acid molecules, preferably they comprise 15 to 40 nucleotides. A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed). The nucleic acid probes may be used to detect KLK12, KLK14, and/or KLK15 nucleic acids, preferably in human cells. The nucleotide probes may also be useful in the diagnosis of endocrine cancer involving KLK12, KLK14, and/or KLK15; in monitoring the progression of such disorder; or monitoring a therapeutic treatment.

The detection of KLK12, KLK14, and/or KLK15 nucleic acid may involve the amplification of specific gene sequences using an amplification method such as polymerase chain reaction (PCR), followed by the analysis of the amplified molecules using techniques known to those skilled in the art. Suitable primers can be routinely designed by one of skill in the art.

By way of example, at least two oligonucleotide primers may be employed in a PCR based assay to amplify a portion of a nucleic acid molecule encoding kallikrein 12, kallikrein 14, and/or kallikrein 15 derived from a sample, wherein at least one of the oligonucleotide primers is specific for (i.e. hybridizes to) a polynucleotide encoding KLK12, KLK14, and/or KLK15. The amplified cDNA is then separated and detected using techniques well known in the art, such as gel electrophoresis.

In order to maximize hybridization under assay conditions, primers and probes employed in the methods of the invention generally have at least about 60%, preferably at least about 75% and more preferably at least about 90% identity to a portion of a polynucleotide encoding kallikrein 12, kallikrein 14, and/or kallikrein 15; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of KLK12, KLK14, and/or KLK15 nucleic acid expression. For example, RNA may be isolated from a cell type or tissue known to express K12, KLK14, and/or KLK15 and tested utilizing the hybridization (e.g. standard Northern analyses) or PCR techniques referred to herein.

The primers and probes may be used in the above-described methods in situ i.e directly on tissue sections (fixed and/or frozen) of patient tissue obtained from biopsies or resections.

In an aspect of the invention, a method is provided employing reverse transcriptase-polymerase chain reaction (RT-PCR), in which PCR is applied in combination with reverse transcription. Generally, RNA is extracted from a sample tissue using standard techinques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against a kallikrein 12, kallikrein 14, and/or kallikrein 15 sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with suspected endocrine cancer and an individual who is not afflicted with endocrine cancer. The reaction may be performed on several dilutions of cDNA spanning at least two orders of magnitude. A statistically signifcant difference in expression in several dilutions of the subject sample as compared to the same dilutions of the non-cancerous sample may be considered positive for the presence of endocrine cancer.

In an embodiment, the invention provides methods for determining the presence or absence of endocrine cancer in a subject comprising (a) contacting a sample obtained from the subject with an oligonucleotide that hybridizes to a nucleic acid molecule encoding kallikrein 14; and (b) detecting in the sample a level of polynucleotide that hybridizes to the nucleic acid molecule relative to a predetermined cut-off value, and therefrom determining the presence or absence of endocrine cancer in the subject.

The invention provides a method wherein kallikrein 14 mRNA is detected by (a) isolating mRNA from a sample and combining the mRNA with reagents to convert it to cDNA; (b) treating the converted cDNA with amplification reaction reagents and nucleic acid primers that hybridize to a nucleic acid molecule encoding kallikrein 14, to produce amplification products; (d) analyzing the amplification products to detect an amount of mRNA encoding kallikrein 14; and (e) comparing the amount of mRNA to an amount detected against a panel of expected values for normal and malignant tissue derived using similar nucleic acid primers.

Kallikrein positive samples or alternatively higher levels, in particular significantly higher levels of kallikrein 14 nucleic acids in patients compared to a control (e.g. cancerous tissue) may be indicative of early stage disease (Grade I or II), optimal debulking, and/or that the patient is responsive to chemotherapy. Kallikrein positive samples or higher levels compared to a control (e.g. cancerous tissue or kallikrein negative samples) may also be indicative of longer progression free disease and overall survival.

In another embodiment, the invention provides methods for determining the presence or absence of endocrine cancer in a subject comprising (a) contacting a sample obtained from the subject with an oligonucleotide that hybridizes to a nucleic acid molecule encoding kallikrein 15; and (b) detecting in the sample a level of polynucleotide that hybridizes to the nucleic acid molecule relative to a predetermined cut-off value, and therefrom determining the presence or absence of endocrine cancer in the subject The invention provides a method wherein kallikrein 15 mRNA is detected by (a) isolating mRNA from a sample and combining the mRNA with reagents to convert it to cDNA; (b) treating the converted cDNA with amplification reaction reagents and nucleic acid primers that hybridize to a nucleic acid molecule encoding kallikrein 15, to produce amplification products; (d) analyzing the amplification products to detect an amount of mRNA encoding kallikrein 15; and (e) comparing the amount of mRNA to an amount detected against a panel of expected values for normal and malignant tissue derived using similar nucleic acid primers.

Kallikrein positive samples or alternatively higher levels, in particular significantly higher levels of kallikrein 15 nucleic acids in patients compared to a control (e.g. normal or benign) are indicative of endocrine cancer. Kallikrein positive samples or higher levels compared to a control (e.g. normal or benign) may also be indicative of reduced progression free disease and overall survival.

Oligonucleotides or longer fragments derived from a KLK12, KLK14, and/or KLK15 nucleic acid may be used as targets in a microarray. The microarray can be used to simultaneously monitor the expression levels of large numbers of genes and to identify genetic variants, mutations, and polymorphisms. The information from the microarray may be used to determine gene function, to understand the genetic basis of a disorder, to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.)

Thus, the invention also includes an array comprising a KLK12, KLK14, and/or KLK15 marker, and optionally other cancer markers. The array can be used to assay expression of KLK12, KLK14, and/or KLK15 in the array. The invention allows the quantitation of expression of kallikrein 12, kallikrein 14, and/or kallikrein 15.

In an embodiment, the array can be used to monitor the time course of expression of KLK12, KLK14, and/or KLK15 polynucleotides in the array. This can occur in various biological contexts such as tumor progression.

The array is also useful for ascertaining differential expression patterns of KLK12, KLK14, and/or KLK15 polynucleotides, and optionally other cancer markers, in normal and abnormal cells. This may provide a battery of nucleic acids that could serve as molecular targets for diagnosis or therapeutic intervention.

Protein Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of a cancer in a subject may be determine by (a) contacting a sample from the subject with a binding agent; (b) detecting in the sample a level of polypeptide that binds to the binding agent; and (c) comparing the level of polypeptide with a predetermined standard or cut-off value.

In particular embodiments of the invention, the binding agent is an antibody.

In an aspect, the invention provides a diagnostic method for monitoring or diagnosing endocrine cancer in a subject by quantitating kallikrein 12, kallikrein 14, and/or kallikrein 15 in a biological sample from the subject comprising reacting the sample with antibodies specific for kallikrein 12, kallikrein 14, and/or kallikrein 15, which are directly or indirectly labeled with detectable substances and detecting the detectable substances. In a particular embodiment of the invention, kallikrein 14 is quantitated or measured.

In an aspect of the invention, a method for detecting endocrine cancer is provided comprising:
(a) obtaining a sample suspected of containing kallikrein 12, kallikrein 14, and/or kallikrein 15 associated with prostate cancer,
(b) contacting said sample with antibodies that specifically bind kallikrein 12, kallikrein 14, and/or kallikrein 15 under conditions effective to bind the antibodies and form complexes;
(c) measuring the amount of kallikrein 12, kallikrein 14, and/or kallikrein 15 present in the sample by quantitating the amount of the complexes; and
(d) comparing the amount of kallikrein 12, kallikrein 14, and/or kallikrein 15 present in the samples with the amount of kallikrein 12, kallikrein 14, and/or kallikrein 15 in a control, wherein a change or significant difference in the amount of kallikrein 12, kallikrein 14, and/or kallikrein 15 in the sample compared with the amount in the control is indicative of endocrine cancer.

In an embodiment, the invention contemplates a method for monitoring the progression of endocrine cancer in an individual, comprising:
(a) contacting antibodies which bind to kallikrein 12, kallikrein 14, and/or kallikrein 15 with a sample from the individual so as to form complexes comprising the antibodies and kallikrein 12, kallikrein 14, and/or kallikrein 15 in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of disease, disease stage, and/or progression of the cancer in said individual.

The amount of complexes may also be compared to a value representative of the amount of the complexes from an individual not at risk of, or afflicted with, endocrine cancer at different stages. A significant difference in complex formation may be indicative of advanced disease e.g. advanced endocrine cancer, or an unfavourable prognosis.

In embodiments of the methods of the invention, kallikrein 14 is detected in samples and higher levels, in particular significantly higher levels compared to a control (normal or benign) is indicative of endocrine cancer or breast cancer.

Antibodies specifically reactive with a kallikrein 12, kallikrein 14, and/or kallikrein 15 protein, or derivatives, such as enzyme conjugates or labeled derivatives, may be used to detect kallikrein 12, kallikrein 14, and/or kallikrein 15 protein in various samples (e.g. biological materials). They may be used as diagnostic or prognostic reagents and they may be used to detect abnormalities in the level of kallikrein 12, kallikrein 14, and/or kallikrein 15 expression, or abnormalities in the structure, and/or temporal, tissue, cellular, or subcellular location of kallikrein 12, kallikrein 14, and/or kallikrein 15. Antibodies may also be used to screen potentially therapeutic compounds its vitro to determine their effects on disorders (e.g. endocrine cancer) involving a kallikrein 12, kallikrein 14, and/or kallikrein 15 protein, and other conditions. In vitro immunoassays may also be used to assess or monitor the efficacy of particular therapies.

Antibodies may be used in any known immunoassays that rely on the binding interaction between antigenic determinants of kallikrein 12, kallikrein 14, and/or kallikrein 15 and the antibodies. Immunoassay procedures for in vitro detection of antigens in fluid samples are also well known in the art [See for example, Paterson et al., Int J. Can. 37:659 (1986) and Burchell et al., Int. J. Can. 34:763 (1984) for a general description of immunoassay procedures]. Qualitative and/or quantitative determinations of kallikrein 12, kallikrein 14, and/or kallikrein 15 in a sample may be accomplished by competitive or non-competitive immunoassay procedures in either a direct or indirect format. Detection of kallikrein 12, kallikrein 14, and/or kallikrein 15 using antibodies can be done utilizing immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

According to an embodiment of the invention, an immunoassay for detecting kallikrein 12, kallikrein 14, and/or kallikrein 15 in a biological sample comprises contacting binding agents that specifically bind to kallikrein 12, kallikrein 14, and/or kallikrein 15 in the sample under conditions that allow the formation of first complexes comprising a binding agent and kallikrein 12, kallikrein 14, and/or kallikrein 15 and determining the presence or amount of the complexes as a measure of the amount of kallikrein 12, kallikrein 14, and/or kallikrein 15 contained in the sample. In a particular embodiment, the binding agents for kallikrein 12, kallikrein 14, and/or kallikrein 15 are labeled differently or are capable of binding to different labels.

Antibodies may be used to detect and quantify kallikrein 12, kallikrein 14, and/or kallikrein 15 in a sample in order to diagnose and treat pathological states. In particular, the antibodies may be used in immunohistochemical analyses, for example, at the cellular and sub-subcellular level, to detect kallikrein 12, kallikrein 14, and/or kallikrein 15 proteins, to localize them to particular endocrine tumor cells and tissues, and to specific subcellular locations, and to quantitate the level of expression.

Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a tissue sample obtained from a subject suspected of having an endocrine-related problem is contacted with antbodies, preferably monoclonal antibodies recognizing kallikrein 12, kallikrein 14, and/or kallikrein 15. The site at which the antibodies are bound is determined by selective staining of the sample by standard immunohistochemical procedures. The same procedure may be repeated on the same sample using other antibodies that recognize kallikrein 12, kallikrein 14, and/or kallikrein 15. Alternatively, a sample may be contacted with antibodies against kallikrein 12, kallikrein 14, and/or kallikrein 15 simultaneously, provided that the antibodies are labeled differently or are able to bind to a different label. In one embodiment of the present invention, the tissue sample is obtained from the ovaries of a patient. The tissue sample may be normal endocrine tissue, a cancer tissue or a benign tissue.

In a sandwich immunoassay of the invention mouse polyclonal antibodies and rabbit polyclonal antibodies are utilized.

Antibodies specific for kallikrein 12, kallikrein 14, and/or kallikrein 15 may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways an antibody can be detectably labeled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steriod isomerase, yeast alcohol dehydrogenase, alpha-glycerophosphate, triose phosphate isomerase, asparaginase, glucose oxidase, and acetylcholine esterase.

For increased sensitivity in an immunoassay system a fluorescence-emitting metal atom such as Eu (europium) and other lanthanides can be used. These can be attached to the desired molecule by means of metal-chelating groups such as DTPA or EDTA.

A bioluminescent compound may also be used as a detectable substance. Bioluminescence is a type of chemiluminescence found in biological systems where a catalytic protein increases the efficiency of the chemiluminescent reaction. The presence of a bioluminescent molecule is determined by detecting the presence of luminescence. Examples of bioluminescent detectable substances are luciferin, luciferase and aequorin.

Indirect methods may also be employed in which the primary antigen-antibody reaction is amplified by the introduction of a second antibody, having specificity for the antibody reactive against kallikrein 12, kallikrein 14, and/or kallikrein 15. By way of example, if the antibody having specificity against kallikrein 12, kallikrein 14, and/or kallikrein 15 is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect a kallikrein 12, kallikrein 14, and/or kallikrein 15 protein Generally, an antibody may be labeled with a detectable substance and kallikrein 12, kallikrein 14, and/or kallikrein 15 protein may be localised in tissues and cells based upon the presence of the detectable substance.

In the context of the methods of the invention, the sample, binding agents (e.g an antibodies specific for kallikrein 12, kallikrein 14, and/or kallikrein 15), or kallikrein 12, kallikrein 14, and/or kallikrein 15 may be immobilized on a carrier or support. Examples of suitable carriers or supports are agarose, cellulose, nitrocellulose, dextran, Sephadex, Sepharose, liposomes, carboxymethyl cellulose, polyacrylamaides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling. An anti-hK5 antibody may be indirectly immobilized using a second antibody specific for the antibody. For example, mouse anti-hK5 antibody may be immobilized using sheep anti-mouse IgG, Fc fragment specific antibody coated on the carrier or support.

Where a radioactive label is used as a detectable substance, a kallikrein 12, kallikrein 14, and/or kallikrein 15 protein may be localized by radioautography. The results of radioautography may be quantitated by determining the density of particles in the radioautographs by various optical methods, or by counting the grains.

Time-resolved fluorometry may be used to detect a signal. For example, the method described in Christopoulos T K and Diamandis E P Anal Chem 1992:64:342-346 may be used with a conventional time-resolved fluorometer.

In accordance with an embodiment of the invention, a method is provided wherein kallikrein 12, kallikrein 14, and/or kallikrein 15 antibodies are directly or indirectly labelled with enzymes, substrates for the enzymes are added wherein the substrates are selected so that the substrates, or a reaction product of an enzyme and substrate, form fluorescent complexes with a lanthanide metal (e.g. europium, terbium, samarium, and dysprosium, preferably europium and terbium). A lanthanide metal is added and kallikrein 12, kallikrein 14, and/or kallikrein 15 are quantitated in the sample by measuring fluorescence of the fluorescent complexes. Enzymes are selected based on the ability of a substrate of the enzyme, or a reaction product of the enzyme and substrate, to complex with lanthanide metals such as europium and terbium. Suitable enzymes and substrates that provide fluorescent complexes are described in U.S. Pat. No. 5,3112,922 to Diamandis. Examples of suitable enzymes include alkaline phosphatase and β-galactosidase. Preferably, the enzyme is alkaline phosphatase.

Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer e.g. a CyberFluor 615 Imunoanalyzer (Nordion International, Kanata, Ontario).

The kallikrein 12, kallikrein 14, and/or kallikrein 15 antibodies may also be indirectly labelled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment, the antibodies are biotinylated, and the enzyme is coupled to streptavidin. In another embodiment, an antibody specific for the anti-kallikrein 12, kallikrein 14, and/or kallikrein 15 antibody is labeled with an enzyme.

In accordance with an embodiment, the present invention provides means for determining kallikrein 12, kallikrein 14, and/or kallikrein 15 in a sample, in particular a serum sample, by measuring kallikrein 12, kallikrein 14, and/or kallikrein 15 by immunoassay. It will be evident to a skilled artisan that a variety of immunoassay methods can be used to measure kallikrein 12, kallikrein 14, and/or kallikrein 15 in serum. In general, a kallikrein 12, kallikrein 14, and/or kallikrein 15 immunoassay method may be competitive or noncompetitive. Competitive methods typically employ an immobilized or immobilizable antibody to kallikrein 12, kallikrein 14, or kallikrein 15 (anti-kallikrein 12, kallikrein 14, or kallikrein 15) and form of kallikrein 12, kallikrein 14, or kallikrein 15. Sample kallikrein 12, kallikrein 14, or kallikrein 15 and labeled kallikrein 12, kallikrein 14, or kallikrein 15 compete for binding to anti-kallikrein 12, kallikrein 14, or kallikrein 15. After separation of the resulting labeled kallikrein 12, kallikrein 14, or kallikrein 15 that has become bound to anti-kallikrein 12, kallikrein 14, or kallikrein 15 (bound fraction) from that which has remained unbound (unbound fraction), the amount of the label in either bound or unbound fraction is measured and may be correlated with the amount of kallikrein 12, kallikrein 14, or kallikrein 15 in the test sample in any conventional manner, e.g., by comparison to a standard curve.

In an aspect, a non-competitive method is used for the determination of kallikrein 12, kallikrein 14, or kallikrein 15, with the most common method being the “sandwich” method. In this assay, two anti-kallikrein 12, kallikrein 14, or kallikrein 15 antibodies are employed. One of the anti-kallikrein 12, kallikrein 14, or kallikrein 15 antibodies is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally it is measured in the capture antibody phase since it comprises kallikrein 12, kallikrein 14, or kallikrein 15 bound by (“sandwiched” between) the capture and detection antibodies. In an embodiment, the label may be measured without separating the capture antibodies and liquid test mixture.

In a typical two-site immunometric assay for kallikrein 12, kallikrein 14, or kallikrein 15, one or both of the capture and detection antibodies are polyclonal antibodies or one or both of the capture and detection antibodies are monoclonal antibodies (ie. polyclonal/polyclonal, monoclonal/monoclonal, or monoclonal/polyclonal). The label used in the detection antibody can be selected from any of those known conventionally in the art. The label may be an enzyme or a chemiluminescent moiety, but it can also be a radioactive isotope, a fluorophor, a detectable ligand (eg., detectable by a secondary binding by a labeled binding partner for the ligand), and the like. Preferably the antibody is labelled with an enzyme which is detected by adding a substrate that is selected so that a reaction product of the enzyme and substrate forms fluorescent complexes. The capture antibody may be selected so that it provides a means for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

A particular sandwich immunoassay method of the invention employs two antibodies reactive against kallikrein 12, kallikrein 14, or kallikrein 15, a second antibody having specificity against an antibody reactive against kallikrein 12, kallikrein 14, or kallikrein 15 labelled with an enzymatic label, and a fluorogenic substrate for the enzyme. In an embodiment the enzyme is alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb3+ fluorescence in a time-resolved mode. Fluorescence intensity is measured using a time-resolved fluorometer as described herein.

The above-described immunoassay methods and formats are intended to be exemplary and are not limiting.

Computer Systems

Computer readable media comprising kallikrein 12, kallikrein 14, and/or kallikrein 15, and/or nucleic acids encoding kallikrein 12, kallikrein 14, and/or kallikrein 15, and optionally other markers of endocrine cancer is also provided. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on kallikrein 12, kallikrein 14, and/or kallikrein 15, and optionally other endocrine cancer markers.

A variety of data processor programs and formats can be used to store information on kallikrein 12, kallikrein 14, and/or kallikrein 15, and/or nucleic acids encoding kallikrein 12, kallikrein 14, and/or kallikrein 15, and other endocrine cancer markers on computer readable medium For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has endocrine cancer or a pre-disposition to endocrine cancer, comprising determining the presence or absence of kallikrein 12, kallikrein 14, and/or kallikrein 15, and/or nucleic acids encoding kallikrein 12, kallikrein 14, and/or kallikrein 15, and optionally other endocrine cancer markers, and based on the presence or absence of the kallikrein 12, kallikrein 14, and/or kallikrein 15, and/or nucleic acids encoding kallikrein 12, kallikrein 14, and/or kallikrein 15, and optionally other markers, determining whether the patient has endocrine cancer or a pre-disposition to endocrine cancer, and optionally recommending treatment for the endocrine cancer or pre-disease condition.

The invention also provides in an electronic system and/or in a network, a method for determining whether a subject has endocrine cancer or a pre-disposition to endocrine cancer, comprising determining the presence or absence of kallikrein 12, kallikrein 14, and/or kallikrein 15, and/or nucleic acids encoding kallikrein 12, kallikrein 14, and/or kallikrein 15, and optionally other endocrine cancer markers, and based on the presence or absence of the kallikrein 12, kallikrein 14, and/or kallikrein 15, and/or nucleic acids encoding kallikrein 12, kallikrein 14, and/or kallikrein 15, and optionally other markers, determining whether the subject has endocrine cancer or a pre-disposition to endocrine cancer, and optionally recommending treatment for the an endocrine cancer or pre-disease condition.

The invention further provides in a network, a method for determining whether a subject has endocrine cancer or a pre-disposition to endocrine cancer comprising: (a) receiving phenotypic information on the subject and information on kallikrein 12, kallikrein 14, and/or kallikrein 15, and/or nucleic acids encoding kallikrein 12, kallikrein 14, and/or kallikrein 15, and optionally other endocrine cancer markers associated with samples from the subject; (b) acquiring information from the network corresponding to the kallikrein 12, kallikrein 14, and/or kallikrein 15., and/or nucleic acids encoding kallikrein 12, kallikrein 14, and/or kallikrein 15, and optionally other markers; and (c) based on the phenotypic information and information on the kallikrein 12, kallikrein 14, and/or kallikrein 15, and/or nucleic acids encoding kallikrein 12, kallikrein 14, and/or kallikrein 15, and optionally other markers determining whether the subject has endocrine cancer or a predisposition to endocrine cancer, and (d) optionally recommending treatment for the endocrine cancer or pre-disease condition.

The invention still further provides a system for identifying selected records that identify an endocrine cancer cell or tissue. A system of the invention generally comprises a digital computer, a database server coupled to the computer; a database coupled to the database server having data stored therein, the data comprising records of data comprising kallikrein 12, kallikrein 14, and/or kallikrein 15, and/or nucleic acids encoding kallikrein 12, kallikrein 14, and/or kallikrein 15, and optionally other endocrine cancer markers, and a code mechanism for applying queries based upon a desired selection criteria to the data file in the database to produce reports of records which match the desired selection criteria.

In an aspect of the invention a method is provided for detecting endocrine cancer tissue or cells using a computer having a processor, memory, display, and input/output devices, the method comprising the steps of:
(a) creating records of kallikrein 12, kallikrein 14, and/or kallikrein 15, and/or nucleic acids encoding kallikrein 12, kallikrein 14, and/or kallikrein 15, and optionally other endocrine cancer markers isolated from a sample suspected of containing endocrine cancer cells or tissue;
(b) providing a database comprising records of data comprising kallikrein 12, kallikrein 14, and/or kallikrein 15, and/or nucleic acids encoding kallikrein 12, kallikrein 14, and/or kallikrein 15, and optionally other endocrine cancer markers; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from cells or tissue that are endocrine cancer cells or tissue.

The invention contemplates a business method for determining whether a subject has endocrine cancer or a pre-disposition to endocrine cancer comprising: (a) receiving phenotypic information on the subject and information on kallikrein 12, kallikrein 14, and/or kallikrein 15, and/or nucleic acids encoding kallikrein 12, kallikrein 14, and/or kallikrein 15, and optionally other endocrine cancer markers associated with samples from the subject; (b) acquiring information from a network corresponding to kallikrein 12, kallikrein 14, and/or kallikrein 15, and/or nucleic acids encoding kallikrein 12, kallikrein 14, and/or kallikrein 15, and optionally other markers; and (c) based on the phenotypic information, information on kallikrein 12, kallikrein 14, and/or kallikrein 15, and/or nucleic acids encoding kallikrein 12, kallikrein 14, and/or kallikrein 15, and optionally other markers, and acquired information, determining whether the subject has endocrine cancer or a pre-disposition to endocrine cancer, and (d) optionally recommending treatment for the endocrine cancer or precondition.

In an aspect of the invention, the computer systems, components, and methods described herein are used to monitor disease or determine the stage of disease.

Imaging Methods

Binding agents, in particular antibodies, specific for kallikrein 12, kallikrein 14, and/or kallikrein 15 may also be used in imaging methodologies in the management of endocrine cancer. The invention provides a method for imaging tumors associated with one or more kallikreins, preferably kallikreins associated with endocrine cancer, most preferably kallikrein 12, kallikrein 14, and/or kallikrein 15 and optionally kallikrein 4, kallikrein 5, kallikrein 6, kallikrein 8, kallikrein 9, kallikrein 10, and kallikrein 11.

The invention also contemplates imaging methods described herein using multiple markers for endocrine cancer. For example, a method for imaging endocrine cancer may further comprise injecting the patient with one or more of an agent that binds to human stratum corneum chymotryptic enzyme (HSCCE), kallikrein 4, kallikrein 5, kallikrein 8, kallikrein 9, kallikrein 10,CA125, CA15.3, CA19-9, CA72-4, inhibin, prostasin, OVX1, LASA, lysophosphatidic acid (LPA), or carcinoembryonic antigen (CEA). Preferably each agent is labeled so that it can be distinguished during the imaging.

In an embodiment the method is an in vivo method and a subject or patient is administered one or more agents that carry an imaging label and that are capable of targeting or binding to a kallikrein. The agent is allowed to incubate in vivo and bind to the kallikrein(s) associated with a tumor, preferably endocrine tumors. The presence of the label is localized to the endocrine cancer, and the localized label is detected using imaging devices known to those skilled in the art.

The agent may be an antibody or chemical entity that recognizes the kallikrein(s). In an aspect of the invention the agent is a polyclonal antibody or monoclonal antibody, or fragments thereof, or constructs thereof including but not limited to, single chain antibodies, bifunctional antibodies, molecular recognition units, and peptides or entities that mimic peptides. The antibodies specific for the kallikreins used in the methods of the invention may be obtained from scientific or commercial sources, or isolated native kallikrein or recombinant kallikrein may be utilized to prepare antibodies etc as described herein.

An agent may be a peptide that mimics the epitope for an antibody specific for a kallikrein and binds to the kallikrein. The peptide may be produced on a commercial synthesizer using conventional solid phase chemistry. By way of example, a peptide may be prepared that includes either tyrosine lysine, or phenylalanine to which N₂S₂ chelate is complexed (See U.S. Pat. No. 4,897,255). The anti-kallikrein peptide conjugate is then combined with a radiolabel (e.g. sodium ^{99m}Tc pertechnetate or sodium ¹⁸⁸Re perrhenate) and it may be used to locate a kallikrein producing tumor.

The agent carries a label to image the kallikreins. The agent may be labelled for use in radionuclide imaging. In particular, the agent may be directly or indirectly labelled with a radioisotope. Examples of radioisotopes that may be used in the present invention are the following: ²⁷⁷Ac, ²¹¹At, ¹²⁸Ba, ¹³¹Ba, ⁷Be, ²⁰⁴ Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, ⁷²Ga, ¹⁹⁸Au, ³H, ¹⁶⁶Ho, ¹¹¹In, ^{113m}In, ^{115m}In, ¹²³I, ¹²⁵I, ¹³¹I, ¹⁸⁹Ir, ^{191m}Ir, ¹⁹²Ir, ¹⁹⁴Ir, ⁵²Fe, ⁵⁵Fe, ⁵⁹Fe, ¹⁷⁷Lu, ¹⁵O, ^{191m-191}Os, ¹⁰⁹Pd, ³²P, ³³P, ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ⁸⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁵Y, ⁹⁰Y, ⁶²Zn and ⁶⁵Zn. Preferably the radioisotope is ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl ⁷⁷Br, or ¹⁸F, and is imaged with a photoscanning device.

Procedures for labeling biological agents with the radioactive isotopes are generally known in the art. U.S. Pat. No. 4,302,438 describes tritium labeling procedures. Procedures for iodinating, tritium labeling, and ³⁵S labeling especially adapted for murine monoclonal antibodies are described by Goding, J. W. (supra, pp. 124-126) and the references cited therein. Other procedures for iodinating biological agents, such as antibodies, binding portions thereof, probes, or ligands, are described in the scientific literature (see Hunter and Greenwood, Nature 144:945 (1962), David et al., Biochemistry 13:1014-1021 (1974), and U.S. Pat. Nos. 3,867,517 and 4,376,110). Iodinating procedures for agents are described by Greenwood, F. et al., Biochem. J. 89:114-123 (1963); Marchalonis, J., Biochem. J. 113:299-305 (1969); and Morrison, M. et al., Immunochemistry, 289-297 (1971). ^{99m}Tc-labeling procedures are described by Rhodes, B. et al. in Burchiel, S. et al. (eds.), Tumor Imaging: The Radioimmunochemical Detection of Cancer, New York: Masson 111-123 (1982) and the references cited therein. Labelling of antibodies or fragments with technetium-99m are also described for example in U.S. Pat. No. 5,317,091, U.S. Pat. No. 4,478,815, U.S. Pat. No. 4,478,818, U.S. Pat. No. 4,472,371, U.S. Pat. No. Re 32,417, and U.S. Pat No. 4,311,688. Procedures suitable for ¹¹¹In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984).

An agent may also be labeled with a paramagnetic isotope for purposes of an in vivo method of the invention. Examples of elements that are useful in magnetic resonance imaging include gadolinium, terbium, tin, iron, or isotopes thereof. (See, for example, Schaefer et al., (1989) JACC 14, 472480; Shreve et al., (1986) Magn. Reson. Med. 3, 336-340; Wolf, G L., (1984) Physiol. Chem. Phys. Med. NMR 16, 93-95; Wesbey et al., (1984) Physiol. Chem. Phys. Med. NMR 16, 145-155; Runge et al., (1984) Invest Radiol. 19, 408-415 for discussions on in vivo nuclear magnetic resonance imaging.)

In the case of a radiolabeled agent, the agent may be administered to the patient, it is localized to the tumor having a kallikrein with which the agent binds, and is detected or “imaged” in vivo using known techniques such as radionuclear scanning using e.g., a gamma camera or emission tomography. [See for example, A. R. Bradwell et al., “Developments in Antibody Imaging”, Monoclonal Antibodies for Cancer Detection and Therapy, R. W. Baldwin et al., (eds.), pp. 65-85 (Academic Press 1985)]. A positron emission transaxial tomography scanner, such as designated Pet VI located at Brookhaven National Laboratory, can also be used where the radiolabel emits positrons (e.g., ¹¹C, ¹⁸F, ¹⁵O, and ¹³N).

Whole body imaging techniques using radioisotope labeled agents can be used for locating both primary tumors and tumors which have metastasized. Antibodies specific for kallikreins, or fragments thereof having the same epitope specificity, are bound to a suitable radioisotope, or a combination thereof, and administered parenterally. For endocrine cancer, administration preferably is intravenous. The bio-distribution of the label can be monitored by scintigraphy, and accumulations of the label are related to the presence of endocrine cancer cells. Whole body imaging techniques are described in U.S. Pat. Nos. 4,036,945 and 4,311,688. Other examples of agents useful for diagnosis and therapeutic use that can be coupled to antibodies and antibody fragments include metallothionein and fragments (see, U.S. Pat. No. 4,732,864). These agents are useful in diagnosis staging and visualization of cancer, in particular endocrine cancer, so that surgical and/or radiation treatment protocols can be used more efficiently.

An imaging agent may carry a bioluminescent or chemiluminescent label. Such labels include polypeptides known to be fluorescent, bioluminescent or chemiluminescent, or, that act as enzymes on a specific substrate (reagent), or can generate a fluorescent, bioluminescent or chemiluminescent molecule. Examples of bioluminescent or chemiluminescent labels include luciferases, aequorin, obelin, mnemippsin, berovin, a phenanthridinium ester, and variations thereof and combinations thereof A substrate for the bioluminescent or chemiluminescent polypeptide may also be utilized in a method of the invention. For example, the chemiluminescent polypeptide can be luciferase and the reagent luciferin. A substrate for a bioluminescent or chemiluminescent label can be administered before, at the same time (e.g., in the same formulation), or after administration of the agent.

An imaging agent may comprise a paramagnetic compound, such as a polypeptide chelated to a metal, e.g., a metalloporphyrin. The paramagnetic compound may also comprise a monocrystalline nanoparticle, e.g., a nanoparticle comprising a lanthanide (e.g., Gd) or iron oxide; or, a metal ion comprising a lanthanide. “Lanthanides” refers to elements of atomic numbers 58 to 70, a transition metal of atomic numbers 21 to 29, 42 or 44, a Gd(III), a Mn(II), or an element comprising an Fe element Paramagnetic compounds can also comprise a neodymium iron oxide (NdFeO.sub.3) or a dysprosium iron oxide (DyFeO.sub.3). Examples of elements that are useful in magnetic resonance imaging include gadolinium, terbium, tin, iron, or isotopes thereof. (See, for example, Schaefer et al., (1989) JACC 14, 472-480; Shreve et al., (1986) Magn. Reson. Med. 3, 336-340; Wolf, G L, (1984) Physiol. Chem. Phys. Med. NMR 16, 93-95; Wesbey et al., (1984) Physiol. Chem. Phys. Med. NMR 16, 145-155; Runge et al., (1984) Invest Radiol. 19, 408-415 for discussions on in vivo nuclear magnetic resonance imaging.)

An image can be generated in a method of the invention by computer assisted tomography (CAT), magnetic resonance spectroscopy (MRS) image, magnetic resonance imaging (MRI), positron emission tomography (PET), single-photon emission computed tomography (SPECT), or bioluminescence imaging (BLI) or equivalent.

Computer assisted tomography (CAT) and computerized axial tomography (CAT) systems and devices well known in the art can be utilized in the practice of the present invention. (See, for example, U.S. Pat. Nos. 6,151,377; 5,946,371; 5,446,799; 5,406,479; 5,208,581; 5,109,397). The invention may also utilize animal imaging modalities, such as MicroCAT.TM. (ImTek, Inc.).

Magnetic resonance imaging (MRI) systems and devices well known in the art can be utilized in the practice of the present invention. In magnetic resonance methods and devices, a static magnetic field is applied to a tissue or a body in order to define an equilibrium axis of magnetic alignment in a region of interest. A radio frequency field is then applied to the region in a direction orthogonal to the static magnetic field direction to excite magnetic resonance in the region. The resulting radio frequency signals are then detected and processed, and the exciting radio frequency field is applied. The resulting signals are detected by radio-frequency coils that are placed adjacent to the tissue or area of the body of interest (For a description of MRI methods and devices see, for example, U.S. Pat. Nos. 6,151,377; 6,144,202; 6,128,522; 6,127,825; 6,121,775; 6,119,032; 6,115,446; 6,111,410; 602,891; 5,555,251; 5,455,512; 5,450,010; 5,378,987; 5,214,382; 5,031,624; 5,207,222; 4,985,678; 4,906,931; 4,558,279). MRI and supporting devices are commercially available for example, from Bruker Medical GMBH; Caprius; Esaote Biomedica; Fonar; GE Medical Systems (GEMS); Hitachi Medical Systems America; Intermagnetics General Corporation; Lunar Corp.; MagneVu; Marconi Medicals; Philips Medical Systems; Shimadzu; Siemens; Toshiba America Medical Systems; including imaging systems, by, e.g., Silicon Graphics. The invention may also utilize animal imaging modalities such as micro-MRIs.

Positron emission tomography imaging (PET) systems and devices well known in the art can be utilized in the practice of the present invention. For example, a method of the invention may use the system designated Pet VI located at Brookhaven National Laboratory. For descriptions of PET systems and devices see, for example, U.S. Pat. Nos. 6,151,377; 6,072,177; 5,900,636; 5,608,221; 5,532,489; 5,272,343; 5,103,098. Animal imaging modalities such as micro-PETs (Corcorde Microsystems, Inc.) can also be used in the invention.

Single-photon emission computed tomography (SPECT) systems and devices well known in the art can be utilized in the practice of the present invention. (See, for example, U.S. Pat. Nos. 6,115,446; 6,072,177; 5,608,221; 5,600,145; 5,210,421; 5,103,098.) The methods of the invention may also utilize animal imaging modalities, such as micro-SPECTs.

Bioluminescence imaging includes bioluminescence, fluorescence or chemiluminescence or other photon detection systems and devices that are capable of detecting bioluminescence, fluorescence or chemiluminescence. Sensitive photon detection systems can be used to detect bioluminescent and fluorescent proteins externally, see, for example, Contag (2000) Neoplasia 2:41-52; Zhang (1994) Clin. Exp. Metastasis 12:87-92. The methods of the invention can be practiced using any such photon detection device, or variation or equivalent thereof, or in conjunction with any known photon detection methodology, including visual imaging. By way of example, an intensified charge-coupled device (ICCD) camera coupled to an image processor may be used in the present invention. (See, e.g., U.S. Pat. No. 5,650,135). Photon detection devices are also commercially available from Xenogen, Hamamatsue.

Screening Methods

The invention also contemplates methods for evaluating test agents or compounds for their ability to inhibit endocrine cancer or potentially contribute to endocrine cancer. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The agents or compounds may be endogenous physiological compounds or natural or synthetic compounds.

The invention provides a method for assessing the potential efficacy of a test agent for inhibiting endocrine cancer in a patient, the method comprising comparing:
(a) levels of kallikrein 12, kallikrein 14, and/or kallikrein 15, and/or nucleic acids encoding kallikrein 12, kallikrein 14, and/or kallikrein 15, and optionally other endocrine cancer markers in a first sample obtained from a patient and exposed to the test agent; and
(b) levels of kallikrein 12, kallikrein 14, and/or kallikrein 15 and optionally other markers in a second sample obtained from the patient, wherein the sample is not exposed to the test agent, wherein a significant difference in the levels of expression of kallikrein 12, kallikrein 14, and/or kallikrein 15, and/or nucleic acids encoding kallikrein 12, kallikrein 14, and/or kallikrein 15, and optionally the other markers in the first sample, relative to the second sample, is an indication that the test agent is potentially efficacious for inhibiting endocrine cancer in the patient.

The first and second samples may be portions of a single sample obtained from a patient or portions of pooled samples obtained from a patient.

In an aspect, the invention provides a method of selecting an agent for inhibiting endocrine cancer in a patient comprising:
(a) obtaining a sample from the patient;
(b) separately maintaining aliquots of the sample in the presence of a plurality of test agents;
(c) comparing kallikrein 12, kallikrein 14, and/or kallikrein 15, and/or nucleic acids encoding kallikrein 12, kallikrein 14, and/or kallikrein 15, and optionally other endocrine cancer markers, in each of the aliquots; and
(d) selecting one of the test agents which alters the levels of kallikrein 12, kallikrein 14, and/or kallikrein 15, and/or nucleic acids encoding kallikrein 12, kallikrein 14, and/or kallikrein 15, and optionally other endocrine cancer markers in the aliquot containing that test agent, relative to other test agents.

Still another aspect of the present invention provides a method of conducting a drug discovery business comprising:
(a) providing one or more methods or assay systems for identifying agents that inhibit endocrine cancer in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test compound to contribute to endocrine cancer comprising:
(a) maintaining separate aliquots of cells or tissues from a patient with endocrine cancer in the presence and absence of the test compound; and
(b) comparing kallikrein 12, kallikrein 14, and/or kallikrein 15, and/or nucleic acids encoding kallikrein 12, kallikrein 14, and/or kallikrein 15, and optionally other endocrine cancer markers in each of the aliquots.

A significant difference between the levels of the markers in the aliquot maintained in the presence of (or exposed to) the test compound relative to the aliquot maintained in the absence of the test compound, indicates that the test compound possesses the potential to contribute to endocrine cancer.

Kits

The invention also contemplates kits for carrying out the methods of the invention. Such kits typically comprise two or more components required for performing a diagnostic assay. Components include but are not limited to compounds, reagents, containers, and/or equipment.

The methods described herein may be performed by utilizing pre-packaged diagnostic kits comprising at least one specific KLK12, KLK14, and/or KLK15 nucleic acid or antibody described herein, which may be conveniently used, e.g., in clinical settings to screen and diagnose patients and to screen and identify those individuals exhibiting a predisposition to developing a disorder.

In an embodiment, a container with a kit comprises a binding agent as described herein. By way of example, the kit may contain antibodies or antibody fragments which bind specifically to an epitope of kallikrein 12, kallikrein 14, and/or kallikrein 15 and optionally other endocrine cancer markers, antibodies against the antibodies labelled with an enzyme; and a substrate for the enzyme. The kit may also contain microtiter plate wells, standards, assay diluent, wash buffer, adhesive plate covers, and/or instructions for carrying out a method of the invention using the kit.

In an aspect of the invention, the kit includes antibodies or fragments of antibodies which bind specifically to an epitope of a kallikrein 12, 14, and 15, and means for detecting binding of the antibodies to their epitope associated with tumor cells, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use, usually do not require other reagents, as for example, where the kit contains a radiolabelled antibody preparation for in vivo imaging.

A kit may be designed to detect the level of nucleic acid molecules encoding a kallikrein 12, kallikrein 14, and/or kallikrein 15 in a sample. Such kits generally comprise at least one oligonucleotide probe or primer, as described herein, that hybridizes to a polynucleotide encoding a kallikrein 12, kallikrein 14, and/or kallikrein 15 protein. Such an oligonucleotide may be used, for example, within a PCR or hybridization procedure. Additional components that may be present within the kits include a second oligonucleotide and/or a diagnostic reagent or container to facilitate detection of a polynucleotide encoding a kallikrein 12, kallikrein 14, and/or kallikrein 15 protein.

The reagents suitable for applying the screening methods of the invention to evaluate compounds may be packaged into convenient kits described herein providing the necessary materials packaged into suitable containers.

Thus, the invention relates to a kit for assessing the suitability of each of a plurality of test compounds for inhibiting endocrine cancer in a patient The kit comprises reagents for assessing kallikrein 12, kallikrein 14, and/or kallikrein 15 or nucleic acids encoding same, and optionally a plurality of test agents or compounds.

The invention contemplates a kit for assessing the presence of endocrine cancer cells, wherein the kit comprises antibodies specific for kallikrein 12, kallikrein 14, and/or kallikrein 15, or primers or probes for nucleic acids encoding same, and optionally probes, primers or antibodies specific for other markers associated with endocrine cancer.

Additionally the invention provides a kit for assessing the potential of a test compound to contribute to endocrine cancer. The kit comprises endocrine cancer cells and reagents for assessing kallikrein 12, kallikrein 14, and/or kallikrein 15, nucleic acids encoding same, and optionally other markers associated with endocrine cancer.

Therapeutic Applications

Kallikrein 12, kallikrein 14, and/or kallikrein 15 proteins are targets for endocrine cancer immunotherapy. Immunotherapeutic methods include the use of antibody therapy, in vivo vaccines, and ex vivo immunotherapy approaches.

In one aspect, the invention provides kallikrein 12, kallikrein 14, and/or kallikrein 15 antibodies that may be used systemically to treat endocrine cancer. Preferably antibodies are used that target the tumor cells but not the surrounding non-tumor cells and tissue. Thus, the invention provides a method of treating a patient susceptible to, or having a cancer that expresses kallikrein 12, kallikrein 14, and/or kallikrein 15, comprising administering to the patient an effective amount of an antibody that binds specifically to kallikrein 12, kallikrein 14, and/or kallikrein 15. In another aspect, the invention provides a method of inhibiting the growth of tumor cells expressing kallikrein 12, kallikrein 14, and/or kallikrein 15, comprising administering to a patient an antibody which binds specifically to a kallikrein 12, kallikrein 14, and/or kallikrein 15 in an amount effective to inhibit growth of the tumor cells. Kallikrein 12, kallikrein 14, and/or kallikrein 15 antibodies may also be used in a method for selectively inhibiting the growth of or killing a cell expressing kallikrein 12, kallikrein 14, and/or kallikrein 15 comprising reacting a kallikrein 12, kallikrein 14, and/or kallikrein 15 antibody immunoconjugate or immunotoxin with the cell in an amount sufficient to inhibit the growth of or kill the cell.

By way of example, unconjugated kallikrein 12, kallikrein 14, and/or kallikrein 15 antibody may be introduced into a patient such that the antibody binds to kallikrein 12, kallikrein 14, and/or kallikrein 15 expressing cancer cells and mediates growth inhibition of such cells (including the destruction thereof), and the tumor, by mechanisms which may include complement-mediated cytolysis, antibody-dependent cellular cytotoxicity, altering the physiologic function of kallikrein 12, kallikrein 14, and/or kallikrein 15, and/or the inhibition of ligand binding or signal transduction pathways. In addition to unconjugated kallikrein 12, kallikrein 14, and/or kallikrein 15 antibodies, kallikrein 12, kallikrein 14, and/or kallikrein 15 antibodies conjugated to therapeutic agents (e.g. immunoconjugates) may also be used therapeutically to deliver the agent directly to kallikrein 12, kallikrein 14, and/or kallikrein 15 expressing tumor cells and thereby destroy the tumor. Examples of such agents include abrin, ricin A, Pseudomonas exotoxin, or diphtheria toxin; proteins such as tumor necrosis factor, alpha-interferon, beta-interferon, nerve growth factor, platelet derived growth factor, tissue plasminogen activator, and biological response modifiers such as lymphokines, interleukin-1, interleukin-2, interleukin-6, granulocyte macrophage colony stimulating factor, granulocyte colony stimulating factor, or other growth factors.

Cancer immunotherapy using kallikrein 12, kallikrein 14, and/or kallikrein 15 antibodies may utilize the various approaches that have been successfully employed for cancers, including but not limited to colon cancer (Arlen et al., 1998, Crit Rev Immunol 18: 133-138), multiple myeloma (Ozaki et al., 1997, Blood 90: 3179-3186; Tsunenati et al., 1997, Blood 90: 2437-2444), gastric cancer (Kasprzyk et al., 1992, Cancer Res 52: 2771-2776), B-cell lymphoma (Funakoshi et al., 1996, J Immunther Emphasis Tumor Immunol 19: 93-101), leukemia (Zhong et al., 1996, Leuk Res 20: 581-589), colorectal cancer (Moun et al., 1994, Cancer Res 54: 6160-6166); Velders et al., 1995, Cancer Res 55: 4398-4403), and breast cancer (Shepard et al., 1991, J Clin Immunol 11: 117-127).

In the practice of a method of the invention, anti-kallikrein 12, kallikrein 14, and/or kallikrein 15 antibodies capable of inhibiting the growth of cancer cells expressing kallikrein 12, kallikrein 14, and/or kallikrein 15 are administered in a therapeutically effective amount to cancer patients whose tumors express or overexpress kallikrein 12, kallikrein 14, and/or kallikrein 15. The invention may provide a specific, effective and long-needed treatment for endocrine cancer. The antibody therapy methods of the invention may be combined with other therapies including chemotherapy and radiation.

Patients may be evaluated for the presence and level of kallikrein 12, kallikrein 14, and/or kallikrein 15 expression and overexpression in tumors, preferably using immunohistochemical assessments of tumor tissue, quantitative kallikrein 12, kallikrein 14, and/or kallikrein 15 imaging as described herein, or other techniques capable of reliably indicating the presence and degree of kallikrein 12, kallikrein 14, and/or kallikrein 15 expression. Immunohistochemical analysis of tumor biopsies or surgical specimens may be employed for this purpose.

Anti-kallikrein 12, kallikrein 14, and/or kallikrein 15 antibodies useful in treating cancer include those that are capable of initiating a potent immune response against the tumor and those that are capable of direct cytotoxicity. In this regard, anti-kallikrein 12, kallikrein 14, and/or kallikrein 15 antibodies may elicit tumor cell lysis by either complement-mediated or antibody-dependent cell cytotoxicity (ADCC) mechanisms, both of which require an intact Fc portion of the immunoglobulin molecule for interaction with effector cell Fc receptor sites or complement proteins. In addition, anti-kallikrein 12, kallikrein 14, and/or kallikrein 15 antibodies that exert a direct biological effect on tumor growth are useful in the practice of the invention. Such antibodies may not require the complete immunoglobulin to exert the effect. Potential mechanisms by which such directly cytotoxic antibodies may act include inhibition of cell growth, modulation of cellular differentiation, modulation of tumor angiogenesis factor profiles, and the induction of apoptosis. The mechanism by which a particular anti-hK5 antibody exerts an anti-tumor effect may be evaluated using any number of in vitro assays designed to determine ADCC, antibody-dependent macrophage-mediated cytotoxicity (ADMMC), complement-mediated cell lysis, and others known in the art.

The anti-tumor activity of a particular anti-kallikrein 12, kallikrein 14, and/or kallikrein 15 antibody, or combination of anti-kallikrein 12, kallikrein 14, and/or kallikrein 15 antibodies, may be evaluated in vivo using a suitable animal model. Xenogenic cancer models, wherein human cancer explants or passaged xenograft tissues are introduced into immune compromised animals, such as nude or SCID mice, may be employed.

The methods of the invention contemplate the administration of single anti-kallikrein 12, kallikrein 14, and/or kallikrein 15 antibodies as well as combinations, or “cocktails”, of different individual antibodies such as those recognizing different epitopes or other kallikreins. Such cocktails may have certain advantages inasmuch as they contain antibodies that bind to different epitopes or kallikreins and/or exploit different effector mechanisms or combine directly cytotoxic antibodies with antibodies that rely on immune effector functionality. Such antibodies in combination may exhibit synergistic therapeutic effects. In addition, the administration of kallikrein 12, kallikrein 14, and/or kallikrein 15 specific antibodies may be combined with other therapeutic agents, including but not limited to chemotherapeutic agents, androgen-blockers, and immune modulators (e.g., IL2, GM-CSF). The kallikrein 12, kallikrein 14, and/or kallikrein 15 specific antibodies may be administered in their “naked” or unconjugated form, or may have therapeutic agents conjugated to them.

The kallikrein 12, kallikrein 14, and/or kallikrein 15 specific antibodies used in the practice of the method of the invention may be formulated into pharmaceutical compositions comprising a carrier suitable for the desired delivery method. Suitable carriers include any material which when combined with the antibodies retains the anti-tumor function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16.sup.th Edition, A. Osal., Ed., 1980).

Kallikrein 12, kallikrein 14, and/or kallikrein 15 specific antibody formulations may be administered via any route capable of delivering the antibodies to the tumor site. Routes of administration include, but are not limited to, intravenous, intraperitoneal, intramuscular, intratumor, intradermal, and the like. Preferably, the route of administration is by intravenous injection. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of cancer and the severity, grade, or stage of the cancer, the binding affinity and half life of the antibodies used, the degree of kallikrein 12, kallikrein 14, and/or kallikrein 15 expression in the patient, the extent of circulating kallikrein 12, kallikrein 14, and/or kallikrein 15 antigen, the desired steady-state antibody concentration level, frequency of treatment, and the influence of any chemotherapeutic agents used in combination with the treatment method of the invention. Daily doses may range from about 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of a particular antibody necessary to be therapeutically effective in a particular context. Repeated administrations may be required to achieve tumor inhibition or regression. Direct administration of kallikrein 12, kallikrein 14, and/or kallikrein 15 antibodies is also possible and may have advantages in certain situations.

Patients may be evaluated for serum kallikrein 12, kallikrein 14, and/or kallikrein 15 in order to assist in the determination of the most effective dosing regimen and related factors. The kallikrein 12, kallikrein 14, and/or kallikrein 15 assay methods described herein, or similar assays, may be used for quantitating circulating kallikrein 12, kallikrein 14, and/or kallikrein 15 levels in patients prior to treatment. Such assays may also be used for monitoring throughout therapy, and may be useful to gauge therapeutic success in combination with evaluating other parameters such as serum kallikrein 12, kallikrein 14, and/or kallikrein 15 levels.

The invention further provides vaccines formulated to contain a kallikrein 12, kallikrein 14, and/or kallikrein 15 protein or fragment thereof.

In an embodiment, the invention provides a method of vaccinating an individual against kallikrein 14 comprising the step of inoculating the individual with kallikrein 14 or fragment thereof that lacks activity, wherein the inoculation elicits an immune response in the individual thereby vaccinating the individual against kallikrein 14.

The use in anti-cancer therapy of a tumor antigen in a vaccine for generating humoral and cell-mediated immunity is well known and, for example, has been employed in prostate cancer using human PSMA and rodent PAP immunogens (Hodge et al., 1995, Int J. Cancer 63: 231-237; Fong et al., 1997, J. Immunol. 159: 3113-3117). These methods can be practiced by employing a kallikrein 12, kallikrein 14, and/or kallikrein 15 protein, or fragment thereof or a kallikrein 12, kallikrein 14, and/or kallikrein 15-encoding nucleic acid molecule and recombinant vectors capable of expressing and appropriately presenting the kallikrein 12, kallikrein 14, and/or kallikrein 15 immunogen.

By way of example, viral gene delivery systems may be used to deliver a kallikrein 12, kallikrein 14, and/or kallikrein 15 encoding nucleic acid molecule. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding a kallikrein 12, kallikrein 14, and/or kallikrein 15 protein or fragment thereof introduced into the patient (e.g., intramuscularly) to induce an anti-tumor response.

Various ex vivo strategies may also be employed. One approach involves the use of cells to present kallikrein 12, kallikrein 14, and/or kallikrein 15 antigen to a patients immune system. For example, autologous dendritic cells which express MHC class I and II, may be pulsed with kallikrein 12, kallikrein 14, and/or kallikrein 15 or peptides thereof that are capable of binding to MHC molecules, to thereby stimulate cancer (e.g. endocrine cancer) patients' immune systems (See, for example, Tjoa et al., 1996, Prostate 28: 65-69; Murphy et al., 1996, Prostate 29: 371-380).

Anti-idiotypic kallikrein 12, kallikrein 14, and/or kallikrein 15 specific antibodies can also be used in anti-cancer therapy as a vaccine for inducing an immune response to cells expressing a hK5 protein. The generation of anti-idiotypic antibodies is well known in the art and can readily be adapted to generate anti-idiotypic kallikrein 12, kallikrein 14, and/or kallikrein 15 specific antibodies that mimic an epitope on a kallikrein 12, kallikrein 14, and/or kallikrein 15 protein (see, for example, Wagner et al., 1997, Hybridoma 16: 33-40; Foon et al., 1995, J Clin Invest 96: 334-342; Herlyn et al., 1996, Cancer Immunol Immunother 43: 65-76). Such an antibody can be used in anti-idiotypic therapy as presently practiced with other anti-idiotypic antibodies directed against tumor antigens.

Genetic immunization methods may be utilized to generate prophylactic or therapeutic humoral and cellular immune responses directed against cancer cells expressing kallikrein 12, kallikrein 14, and/or kallikrein 15. Using the kallikrein 12, kallikrein 14, and/or kallikrein 15 encoding DNA molecules, constructs comprising DNA encoding a kallikrein 12, kallikrein 14, and/or kallikrein 15 protein/immunogen and appropriate regulatory sequences may be injected directly into muscle or skin of an individual, such that the cells of the muscle or skin take-up the construct and express the encoded kallikrein 12, kallikrein 14, and/or kallikrein 15 protein/immunogen. The kallikrein 12, kallikrein 14, and/or kallikrein 15 protein/immunogen may be expressed as a cell surface protein or be selected. Expression of the kallikrein 12, kallikrein 14, and/or kallikrein 15 protein/immunogen results in the generation of prophylactic or therapeutic humoral and cellular immunity against the cancer. Various prophylactic and therapeutic genetic immunization techniques known in the art may be used.

The invention further provides methods for inhibiting cellular activity (e.g., cell proliferation, activation, or propagation) of a cell expressing kallikrein 12, kallikrein 14, and/or kallikrein 15. This method comprises reacting immunoconjugates of the invention (e.g., a heterogeneous or homogenous mixture) with the cell so that the kallikrein 12, kallikrein 14, and/or kallikrein 15 proteins form a complex with the immunoconjugates. A subject with a neoplastic or preneoplastic condition can be treated when the inhibition of cellular activity results in cell death.

In another aspect, the invention provides methods for selectively inhibiting a cell expressing kallikrein 12, kallikrein 14, and/or kallikrein 15 by reacting any one or a combination of the immunoconjugates of the invention with the cell in an amount sufficient to inhibit the cell. Amounts include those that are sufficient to kill the cell or sufficient to inhibit cell growth or proliferation.

Vectors derived from retroviruses, adenovirus, herpes or vaccinia viruses, or from various bacterial plasmids, may be used to deliver nucleic acid molecules encoding kallikrein 12, kallikrein 14, and/or kallikrein 15 to a targeted organ, tissue, or cell population. Methods well known to those skilled in the art may be used to construct recombinant vectors that will express antisense nucleic acid molecules for kallikrein 12, kallikrein 14, and/or kallikrein 15. (See, for example, the techniques described in Sambrook et al (supra) and Ausubel et al (supra)).

Methods for introducing vectors into cells or tissues include those methods discussed herein and which are suitable for in vivo, in vitro and ex vivo therapy. For cc vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

Genes encoding a kallikrein 12, kallikrein 14, and/or kallikrein 15 protein can be turned off by transfecting a cell or tissue with vectors that express high levels of a desired kallikrein 12, kallikrein 14, and/or kallikrein 15-encoding fragment. Such constructs can inundate cells with untranslatable sense or antisense sequences. Even in the absence of integration into the DNA, such vectors may continue to transcribe RNA molecules until all copies are disabled by endogenous nucleases.

Modifications of gene expression can be obtained by designing antisense molecules, DNA, RNA or PNA, to the regulatory regions of a gene encoding a kallikrein 12, kallikrein 14, and/or kallikrein 15, i.e., the promoters, enhancers, and introns. Preferably, oligonucleotides are derived from the transcription initiation site, eg, between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a kallikrein 12, kallikrein 14, and/or kallikrein 15 protein.

Specific ribozyme cleavage sites within any potential RNA target may initially be identified by scanning the target molecule for ribozyme cleavage sites which include the following sequences, GUA, GWU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

Kallikrein 12, kallikrein 14, and/or kallikrein 15 proteins and nucleic acids encoding the protein, and fragments thereof may be used in the treatment of endocrine cancer in a subject. The proteins or nucleic acids may be formulated into compositions for administration to subjects suffering from endocrine cancer. Therefore, the present invention also relates to a composition comprising a kallikrein 12, kallikrein 14, and/or kallikrein 15 protein or a nucleic acid encoding the protein, or a fragment thereof, and a pharmaceutically acceptable carrier, excipient or diluent. A method for treating or preventing endocrine cancer in a subject is also provided comprising administering to a patient in need thereof, a kallikrein 12, kallikrein 14, and/or kallikrein 15 protein or a nucleic acid encoding the protein, or a composition of the invention.

The invention further provides a method of inhibiting endocrine cancer in a patient comprising:
(a) obtaining a sample comprising diseased cells from the patient;
(b) separately maintaining aliquots of the sample in the presence of a plurality of test agents;
(c) comparing levels of kallikrein 12, kallikrein 14, and/or kallikrein 15, and/or nucleic acids encoding kallikrein 12, kallikrein 14, and/or kallikrein 15 in each aliquot;
(d) administering to the patient at least one of the test agents which alters the levels of the kallikrein 12, kallikrein 14, and/or kallikrein 15, and/or nucleic acids encoding kallikrein 12, kallikrein 14, and/or kallikrein 15 in the aliquot containing that test agent, relative to the other test agents.

The active substance may be administered in a convenient manner such as by injection (subcutaneous, intravenous, etc.), oral administration, inhalation, transdermal application, or rectal administration Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment The compositions of the invention may be administered concurrently, separately, or sequentially with other therapeutic agents or therapies.

The therapeutic activity of compositions and agents/compounds identified using a method of the invention and may be evaluated in vivo using a suitable animal model.

The following non-limiting examples are illustrative of the present invention:

### EXAMPLE 1

KLK14 expression is mainly regulated by androgens and progestins. Kinetic and blocking experiments suggest that this up-regulation is mediated through the androgen receptor. The expression of KLK14 was studied by quantitative RT-PCR in 155 consecutive ovarian tumors and these findings were correlated with clinicopathological parameters, response to chemotherapy and patient survival. A stepwise reduction was observed in the levels of KLK14 mRNA in normal, benign and cancerous tissues (p<0.001). Expression levels were significantly higher in patients with early stage disease, optimal debulking and in patients who responded to chemotherapy. Kaplan-Meier survival curves demonstrated that patients with KLK14-positive tumors have longer progression-free and overall survival, in comparison to those who are KLK14-negative (p<0.001). When all other prognostic variables were controlled in the multivariate analysis, KLK14 retained its prognostic significance (hazard ratios of 0.43 and 0.53 and p values of 0.027 and 0.014 for progression-free and overall survival, respectively). A weak negative correlation was found between KLK14 expression and serum CA125. Thus, KLK14 is a new, independent and favorable prognostic marker for ovarian cancer.

Materials and Methods

Breast Cancer Cell Lines and Hormonal Stimulation Experiments

The breast cancer cell lines BT-474, T-47D, ZR-75, T47D and BT-20 and the ovarian cancer cell line HTB-75(Caov-3) were purchased from the American Type Culture Collection (ATCC), Rockville, Md. The BG-1 ovarian cancer cell line was kindly provided by Dr. Henri Rochefort, Montpellier, France. Cells were cultured in RPMI media (Gibco BRL, Gaithersburg, Md.) supplemented with glutamine (200 mmol/L) and fetal bovine serum (10%), in plastic flasks, to near confluency. The cells were then aliquoted into 24-well tissue culture plates and cultured to 50% confluency. 24 hours before the experiments, the culture medium was changed into medium containing 10% charcoal-stripped fetal bovine serun. For stimulation experiments, various steroid hormones dissolved in 100% ethanol were added into the culture media, at a final concentration of 10⁻⁸ M. Cells stimulated with 100% ethanol were included as controls. The cells were grown for 24 hours, then harvested for mRNA extraction.

Blocking and Kinetic Experiments

Blocking experiments were performed as follows: (a) addition to c-l!red cells of androgen receptor (AR) blockers (i.e. RU56,187 and nilutamide) individually at three different concentrations (10⁻⁶, 10⁻⁷, 10⁻⁸M), (b) stimulation by dihydrotestosterone (DHT) alone at a concentration of 10⁻⁸-10⁻¹⁰M (c) addition of AR blockers at three different concentrations as above for 1 hour, followed by DHT stimulation at 100-fold lower concentration (10⁻⁸-10⁻¹⁰M). Ethanol-only stimulated cells were included as controls to assess baseline KLK14 expression. Cells were harvested for analysis after 24 h.

For kinetic experiments, the BT-474 cell line was stimulated by DHT at a final concentration of 10⁻⁸ M and then harvested at 0 (just before stimulation), 2, 6, 12 and 24 hours. Control cells stimulated with ethanol were included for all time points. All experiments were repeated twice.

Study Population

Included in this study were tumor specimens from 155 consecutive patients undergoing surgical treatment for epithelial ovarian carcinoma at the Department of Gynecology, University of Turin, Turin, Italy. Diagnosis was confirmed by histopathology. Patients received no treatment before surgery.

Patient ages ranged from 19 to 89 with a median of 58 years. Residual tumor size ranged from 0 to 9 cm, with a median of 1.2 cm. With respect to histological type, 70 tumors were serous papillary, 28 were endometrioid, 24 were undifferentiated, 15 were mucinous and 16 were clear cell. For two tumors, histological type was unknown. We also included 10 normal ovarian tissues and 10 tissues from benign ovarian pathologies, from women whose median age was 52 and 54 years, respectively. Classification of histological types followed the World Health Organization criteria (Serov S F, Sorbin L H. Histological typing of ovarian tumors. World Health Organization., 1973). All patients were staged according to the International Federation of Gynecology and Obstetrics (FIGO) staging system (Pettersson F. Annual report on the treatment in gynecological cancer. Stockholm: International Federation of Gynecology and Obstetrics, 1994). Grading information was available for 148 patients; 53 (36%) had grade 1 or 2, while 95 (64%) had grade 3 ovarian carcinoma. Grading was established for each ovarian tumor according to the criteria of Day et al. (Day T G, Jr., Gallager H S, Rutledge F N. Epithelial carcinoma of the ovary-prognostic importance of histologic grade, Natl Canzcer Inst Monogr. 1975;42:15-21). All patients were treated with postoperative platinum-based regimen chemotherapy. The first-line chemotherapy regimens included cisplatin in 87 (56%) patients, carboplatin in 46 (30%), cyclophosphamide in 64 (41%), doxorubicin in 11 (7%), epirubicin in 18 (12%), paclitaxel in 25 (16%), and methotrexate in 2 (1%). Grade 1 and stage I patients receive further treatment. Response to chemotherapy was assessed as follows: complete response was defined as a resolution of all evidence of disease for at least 1 month; a decrease (lasting at least 1 month) of at least 50% in the diameters of all measurable lesions without the development of new lesions was termed partial response. Stable disease was defined as a decrease of <25% in the product of the diameters of all measurable lesions. An increase of at least 25% was termed as progressive disease. Investigations were performed in accordance with the Helsinki declaration and were approved by the Review Board of the Institute of Obstetrics and Gynecology, Turin, Italy. Tumor specimens were snap-frozen in liquid nitrogen immediately after surgery. Histological examination, performed during intra-surgery frozen section analysis, allowed representative portions of each tumor containing >80% tumor cells to be selected for storage until analysis.

Total RNA Extraction and cDNA Synthesis

Samples were shipped and stored at −80° C. They were then minced with a scalpel on dry ice and transferred immediately to 2 ml polypropylene tubes and homogenized. Total RNA was then extracted using Trizol™ reagent (Gibco BRL, Gaithersburg, Md.) following the manufacturer's instructions. The concentration and purity of RNA were determined spectrophotometrically. 2 μg of total RNA were reverse-transcribed into first strand cDNA using the Superscrip™ preamplification system (Gibco BRL). The final volume was 20 μl.

Quantitative Real-Time PCR and Continuous Monitoring of PCR Products

Based on the published genomic sequence of KLK14 (Genbank accession # AF161221), two gene-specific primers were designed [6F5: 5′ AGT GGG TCA TCA CTG CTG CT 3′ (SEQ IID NO. 12) and 6R5: 5′ TCG TTT CCT CAA TCC AGC TT 3′ (SEQ ID NO. 13]. These primers spanned more than 2 exons to avoid contamination by genomic DNA. Real-time monitoring of PCR reaction was performed on the LightCycler™ system (Roche Molecular Systems, Indianapolis, USA) and the SYBR Green I dye, which binds preferentially to double stranded DNA. Fluorescence signals are proportional to the concentration of the product and are measured at the end of each cycle rather than after a fixed number of cycles. The higher the starting quantity of the template, the earlier the threshold cycle, defined as the fractional cycle number at which fluorescence passes a fixed threshold above baseline (Bieche I, et al. Real-time reverse transcription-PCR assay for future management of ERBB2-based clinical applications. Clin Chem. 1999;45:1148-1156). For each sample, the amount of KLK14 and of an endogenous control (β-actin, a housekeeping gene) were determined using a calibration curve (see below). The amount of KLK14 was then divided by the amount of the endogenous reference to obtain a normalized KLK14 value.

Standard Curve Construction

The full-length mRNA sequence of the KLK14 gene was amplified by PCR using gene-specific primers, and the PCR product was cloned into a TOPO TA cloning vector (Invitrogen, Carlsbad, Calif., USA) according to manufacturer's instructions. A plasmid containing β-actin cDNA was prepared similarly. Plasmids were purified using a mini-prep kit (Qiagen Inc., Valencia, Calif.). Different standard curves for actin and KLK14 were constructed using serial dilutions of the plasmid as described elsewhere (Bieche I, et al. Clin Chim. 1999;45:1148-1156). These standards were included in each run.

PCR Amplification

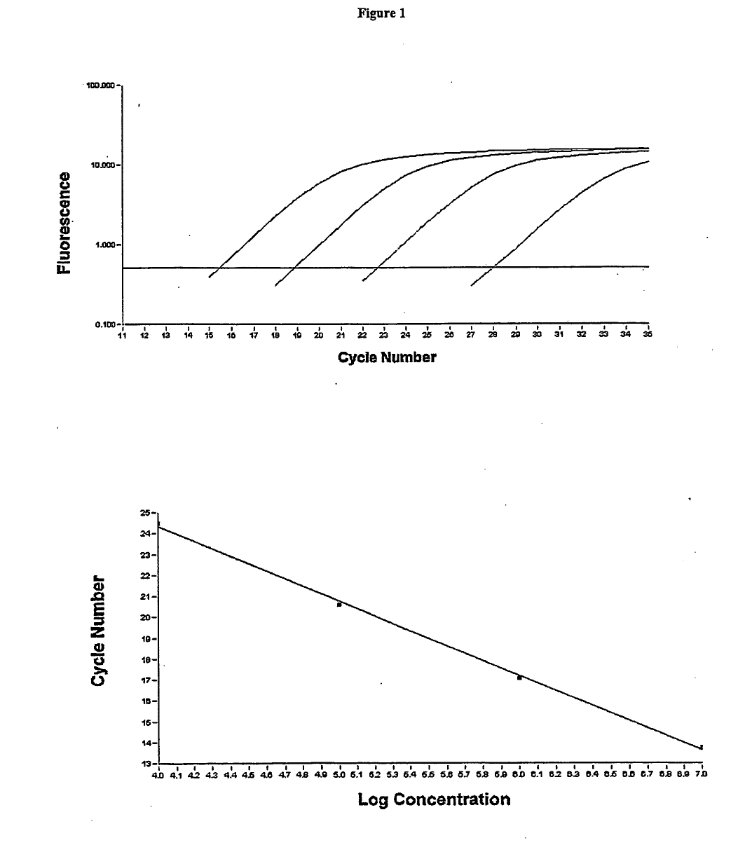

The PCR reaction was carried out on the LightCycler™ system (FIG. 1). For each run, a master mixture containing 1 μl of cDNA, 2 μl of LC DNA Master SYBR Green 1 mix, 50 ng of primers and 2.4 μl of 25 mM MgCl₂ was prepared on ice. After loading the reaction mixture into glass capillary tubes, cycling conditions were carried out as shown in Table 1. To verify the melting curve results, representative PCR products were sequenced.

Statistical Analysis

First, an optimal cutoff value was defined by χ² analysis, based on the ability of KLK14 values to predict the PFS and OS of the study population. This cutoff (1.0 arbitrary units; 50^{th} percentile) identifies 50% of patients as being KLK14 positive.

Associations between clinicopathological parameters such as stage, grade, bistotype, and residual tumor, and KLK14 expression were analyzed by the Chi-square test or the Fisher's Exact Test, where appropriate. For survival analysis, two different end points, cancer relapse (either local recurrence or distant metastasis) and death, were used to calculate progression-free and overall survival, respectively. Progression-free survival was defined as the time interval between the date of surgery and the date of identification of recurrent or metastatic disease. Overall survival was defined as the time interval between the date of surgery and the date of death.

The Cox univariate and multivariate proportional hazard regression model (Cox D R. R Stat Soc. B. 1972;34:187-202) was used to evaluate the hazard ratio (relative risk of relapse or death in the KLK14-positive group). In the multivariate analysis, the models were adjusted for KLK41 expression, clinical stage, histologic grade, residual tumor and age.

Kaplan-Meier survival curves (Kaplan E L, Meier P. J Am Stat Assoc. 1958;53:457-481) were constructed for KLK14-positive and KLK14-negative patients. For further analysis, patients were divided into two groups either by the tumor grade (grade 1-2 vs. grade 3), tumor stage (stage I-II vs stage III-V), or by the success of debulking (optimal vs. suboptimal debulking). In each category, survival rates (disease-free and overall survival) were compared between KLK14-positive and KLK14-negative groups. The differences between survival curves were analyzed by the log rank test (Mantel N. Cancer Chemother Rep. 1966;50:163-170).

Results

Hormonal Regulation of the KLK14 Gene

Sequence analysis of the KLK14 gene promoter using different promoter prediction algorithms indicated the presence of a putative androgen response element [GGAGGCAAGCAGCCTC (SEQ ID NO. 14)] at position −386 (base numbering according to our Genebank accession # AF161221. This element is comparable to the ARE-II of the PSA promoter which is found at approximately the same position. Thus, as is the case with some other kallikreins, KLK14 may be regulated by androgens.

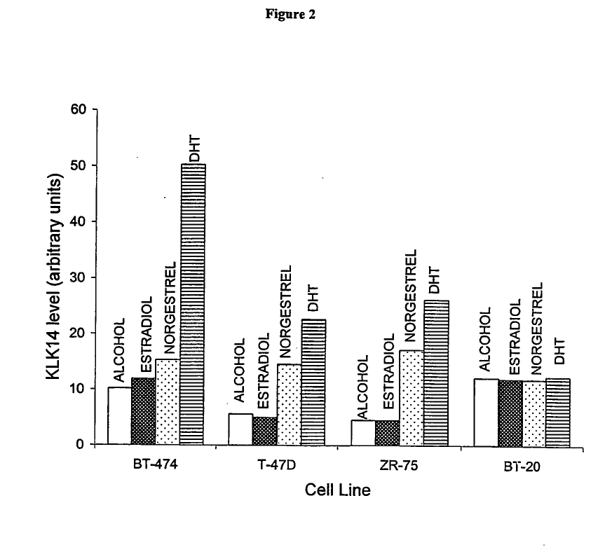

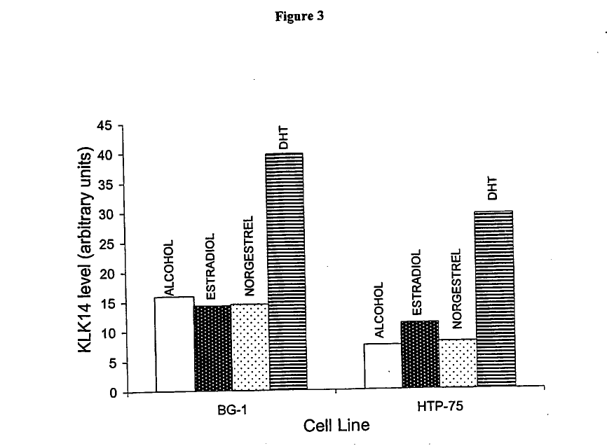

In order to test this, KLK14 gene expression was examined in 4 breast cancer cell lines (BT474, ZR-75, T-47D and BT-20) and two ovarian cancer cell lines (BG-1 and HTB-75(Caov-3)) with variable AR content. Quantitative PCR results indicated that KLK14 was mainly up-regulated by androgens (DHT) in the AR-positive breast cancer cell lines (BT-474, T-47D and ZR-75), and to a lesser extent by progestins (FIG. 2). The gene was also up-regulated by DHT in both ovarian cancer cell lines (FIG. 3). No significant up regulation of KLK14 was found in the AR-negative cell line BT-20 (FIG. 2).

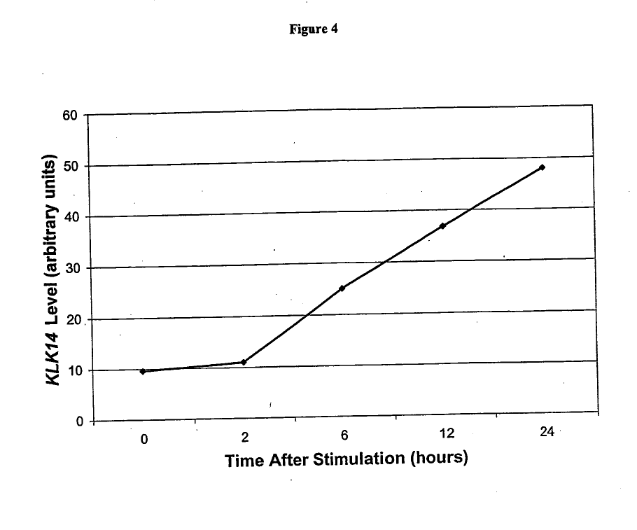

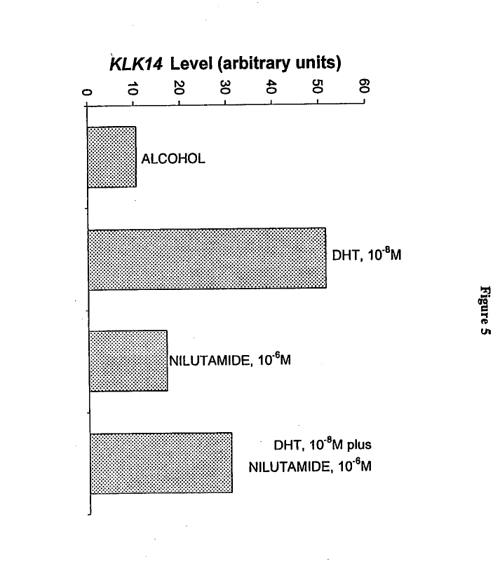

Time-course experiments indicated that KLK14 gene expression starts to increase as early as 2 hours after hormonal stimulation and mRNA levels increase steadily over the next 24 hours (FIG. 4). Results of the blocking experiments are represented in FIG. 5. Both anti-androgens used, RU56,187 and Nilutamide, were added in 100-fold excess prior to DHT stimulation. Nilutamide was able to block the stimulatory effect of DHT by about 50%. RU56,187 had little effect on androgenic stimulation.

KLK 14 Expression in Normal, Benign and Ovarian Cancer Tissues

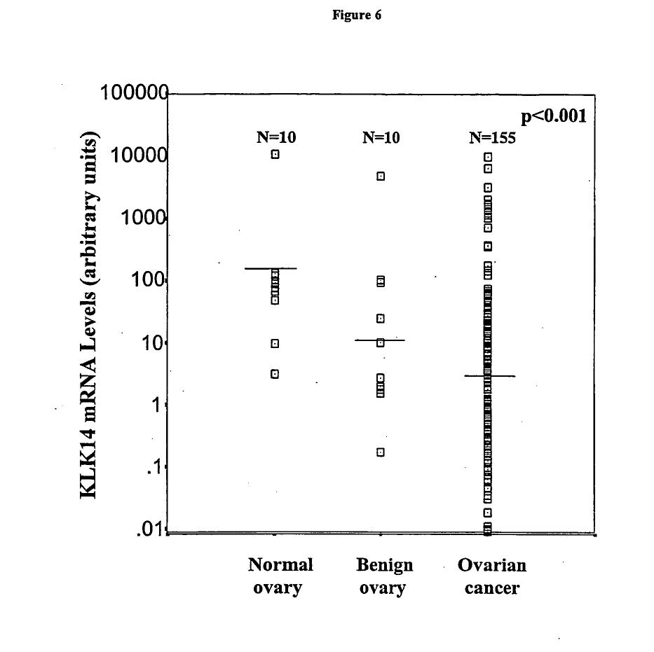

A comparison of KLK14 expression in normal ovarian tissues, benign ovarian tumors and cancerous ovarian tissues is shown in Table 2 and FIG. 6. A stepwise reduction in KLK14 mRNA levels was observed between normal tissues (median=74 arbitrary units), benign ovarian tumors (median=6.5 units), and cancerous tissues (median=1.0 units). These differences were statistically significant (p<0.001).

KLK14 Expression in Relation to Other Variables

As shown in Table 3, KLK14 expression levels were significantly higher in patients with early stage (I or II) disease (p=0.020), in patients with optimal debulking (p=0.020) and in those who responded better to chemotherapy (p=0.008). No significant associations were found between KLK14 expression and grade, histotype, residual tumor and menopause.

Survival Analysis

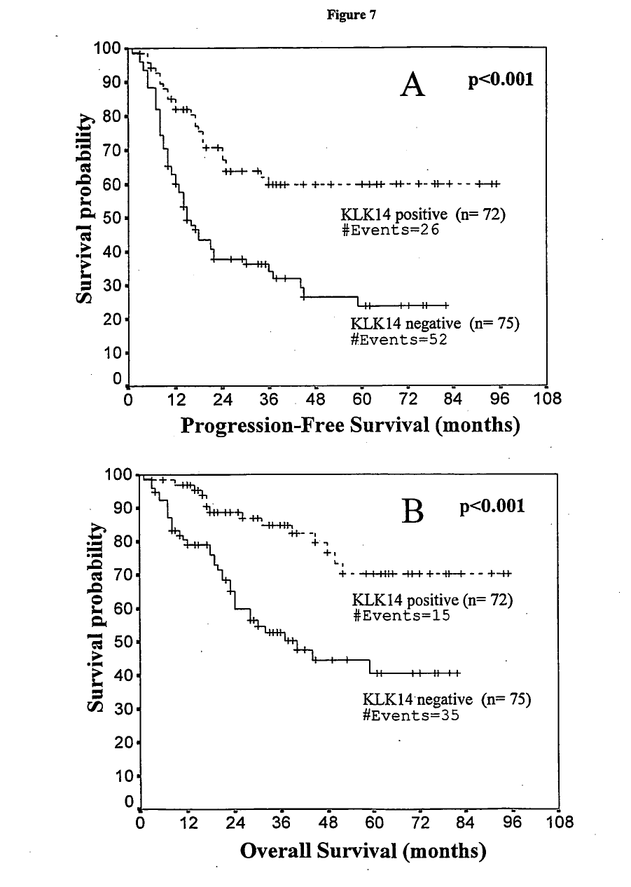

Out of the 155 patients included in this study, follow-up information was available for 147, among whom 79 (54%) relapsed and 50 (34%) died. Kaplan-Meier survival curves demonstrated that patients with KLK14-positive tumors have longer PFS (p<0.001) and overall survival (p<0.001), compared to those who are KLK14-negative (FIG. 7). The strength of the associations between each individual prognostic factor and progression-free or overall survival are shown in the univariate analysis in Table 4. Stage of disease, histological grade and residual tumor size showed strong associations with cancer relapse and death (p<0.001). KLK14 expression was also found to be a significant predictor of prolonged progression-free (PFS) and overall (OS) survival (hazard ratio of 0.38 and 0.33, respectively and p<0.001 for both).

In Cox multivariate analysis, only the residual tumor size and grade, in addition to KLK14, retained their prognostic significance (Table 4).

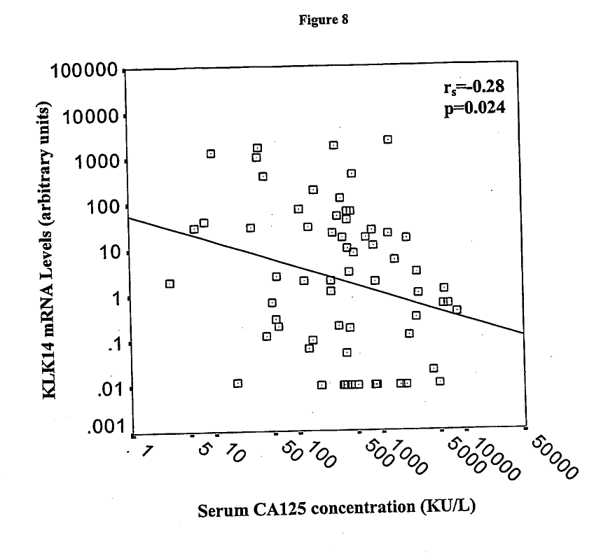

As shown in FIG. 8, a weak negative correlation was found between preoperative serum CA125 and KLK14 mRNA levels (rₛ=−0.28, p=0.024).

Discussion

The results show that KLK14 expression is under steroid hormonal regulation. KLK14 is mainly up-regulated by androgens and progestins in breast and ovarian cancer cell lines. Also provided is some indirect evidence that this regulation might be mediated through the AR. Ovarian cancer is an endocrine-related malignancy, and compelling evidence supports the role of steroid hormones in the development and progression of this disease (Slotman B J, Rao B R. Anticancer Res. 1988;8:417-43; and Risch H A, J Natl Cancer Inst. 1998;90:1774-1786. In addition, the AR gene represents a plausible candidate genetic modifier of ovarian cancer risk (Levine D A, Boyd J. Cancer Res. 2001;61:908-911).Identification of downstream AR-regulated genes is an important initial step towards our understanding of the mechanism by which androgens are implicated in ovarian cancer. These findings may have therapeutic applications.

The results show that KLK14 is an independent marker of favorable prognosis in ovarian cancer (Table 4).

It is now widely accepted that no single biomarker will provide all the necessary information for diagnosis, prognosis and development of treatment strategies in patients with ovarian and other cancers. Instead, research is now focussing on devising panels of ovarian cancer biomarkers. Artificial network and other combinatorial approaches seem to be promising in this regard. (Zhang Z. et al. Gynecol Oncol. 1999;73:56-61; Woolas R P, et al. J Natl Cancer Inst. 1993;85:1748-175; Khan J, et al. Nat Med. 2001;7:673-679; and). In addition, KLK14 might find applicability as a predictive marker of therapy, similar to steroid receptors and hormonal therapy, or HER-2 and Herceptin therapy in breast cancer (Cobleigh M A, et al. J Clin Oncol. 1999; 17:2639-2648).

The data indicates that KLK14 expression levels are negatively correlated with serum CA125 concentration (FIG. 8). These results are consistent with previous reports showing that higher serum CA125 levels are associated with poor prognosis in ovarian cancer (de la Cuesta R, et al., Int J Biol Markers. 1999;14:106-114. On other hand, while high CA125 expression levels are associated with the serous histological type (48), relationships between KLK14 levels and histological type were not found (Table 3).

In conclusion, higher KLK14 expression was shown to have favorable prognostic value in ovarian cancer.

### EXAMPLE 2

KLK15 expression was studied by quantitative RT-PCR in 168 consecutive patients with epithelial ovarian cancer. 10 patients with benign ovarian tumors were also included in the study. An optimal cutoff point equal to the 50^{th} percentile was defined, based on the ability of KLK15 to predict progression-free survival and overall survival of the study population.

KLK15 expression levels were significantly higher in cancerous tissues compared with benign tumors. Kaplan-Meier survival curves showed that KLK15 overexpression is a significant predictor of reduced progression-free (PFS) (p<0.001) and overall survival (OS) (p<0.009). Univariate and multivariate analyses indicate that KLK15 is an independent prognostic factor of PFS and OS. A weak positive correlation was found between KLK15 expression and serum CA125 levels. KLK15 expression, as assessed by quantitative RT-PCR, is an independent marker of unfavorable prognosis for ovarian cancer.

Materials and Methods

Study Population

Included in this study were tumor specimens from 168 consecutive patients undergoing surgical treatment for epithelial ovarian carcinoma at the Department of Gynecology, Gynecological Oncology Unit, University of Turin, Turin, Italy. All tumor specimens were confirmed by histopathology. No patient received any treatment before surgery.

Patient ages ranged from 25 to 89 with a median of 59 years. Residual tumor sizes after surgery ranged from 0 to 9 cm, with a median of 2.0 cm. With respect to histological type, 76 tumors were serous papillary, 28 were endometrioid, 28 were undifferentiated, 17 were mucinous and 15 were clear cell. 10 benign ovarian tissues, from women whose median age was 52 years, were also included. Classification of histological types followed the World Health Organization criteria (Serov S F, Sorbin L H, World Health Organization, 1973).All patients were staged according to the International Federation of Gynecology and Obstetrics staging system (Pettersson F: Annual report on the treatment in gynecological cancer. Stockholm, International Federation of Gynecology and Obstetrics, 1994). Grading information was available for 162 patients; 54 (33%) had grade 1 or 2, while 108 (67%) had grade 3 ovarian carcinoma Grading was established for each ovarian tumor according to the criteria of Day et al. (Nat Cancer Inst Monogr 42:15-21, 1975). All patients were treated with postoperative platinum-based chemotherapy. The first-line chemotherapy regimens included cisplatin in 95 (56%) patients, carboplatin in 50 (30%), cyclophosphamide in 69 (41%), doxorubicin in 12 (7%), epirubicin in 20 (12%), paclitaxel in 27 (16%), and methotrexate in 2 (1%). Grade 1 and stage I patients received no further treatment Response to chemotherapy was assessed as follows: complete response was defined as a resolution of all evidence of disease for at least 1 month; a decrease (lasting at least 1 month) of at least 50% in the diameters of all measurable lesions without the development of new lesions was termed partial response. Stable disease was defined as a decrease of <25% in the product of the diameters of all measurable lesions. An increase of at least 25% was termed as a progressive disease. In patients with no clinically measurable disease, response to chemotherapy was assessed by serial measurements of serum CA125. Responders (partial or complete) reduced their CA125 by more than 50% after 2 cycles of chemotherapy. Investigations were performed in accordance with the Helsinki declaration and approved by the Institute of Obstetrics and Gynecology, Turin, Italy. Tumor specimens were snap-frozen in liquid nitrogen immediately after surgery. Histological examination, performed during intra-surgery frozen section analysis, allowed representative portions of each tumor containing >80% of tumor cells to be selected for storage until analysis. Serum CA125 values before operation were available for 67 patients.

Total RNA Extraction and cDNA Synthesis

Samples were shipped and stored at −80° C. They were then minced with a scalpel on dry ice and transferred immediately to 2 ml polypropylene tubes. They were then homogenized and total RNA was extracted using Trizol™ reagent (Gibco BRL, Gaithersburg, Md.) following the manufacturer's instructions. The concentration and purity of RNA were determined spectrophotometrically. 2 μg of total RNA was reverse-transcribed into first strand cDNA using the Superscript™ preamplification system (Gibco BRL). The final volume was 20 μl.

Quantitative Real-Time PCR and Continuous Monitoring of PCR Products

Based on the published genomic sequence of KLK15 (GenBank accession # AF242195), two gene-specific primers were designed [15-F3: 5′ TGT GGC TTC TCC TCA CTC TC 3′ (SEQ ID NO. 15) and 15-R3 5′AGG CTC GTT GTG GGA CAC 3′ (SEQ ID NO. 16)]. These primers spanned more than 2 exons to avoid contamination by genomic DNA Real-time monitoring of PCR reaction was performed on the LightCycler™ system (Roche Molecular Systems, Indianapolis, USA) and the SYBR Green I dye, which binds preferentially to double stranded DNA. Fluorescence signals are proportional to the concentration of the product and are measured at the end of each cycle rather than after a fixed number of cycles. The higher the starting quantity of the template, the earlier the threshold cycle, defined as the fractional cycle number at which fluorescence passes a fixed threshold above baseline, will be attained (Bieche I, et al., Clin Chem 45:1148-1156, 199). For each sample, the amount of KLK15 and of an endogenous control (β actin, a housekeeping gene) were determined using a calibration curve (see below). The amount of KLK15 was then divided by the amount of the endogenous reference, to obtain a normalized KLK15 value.

Standard Curve Construction

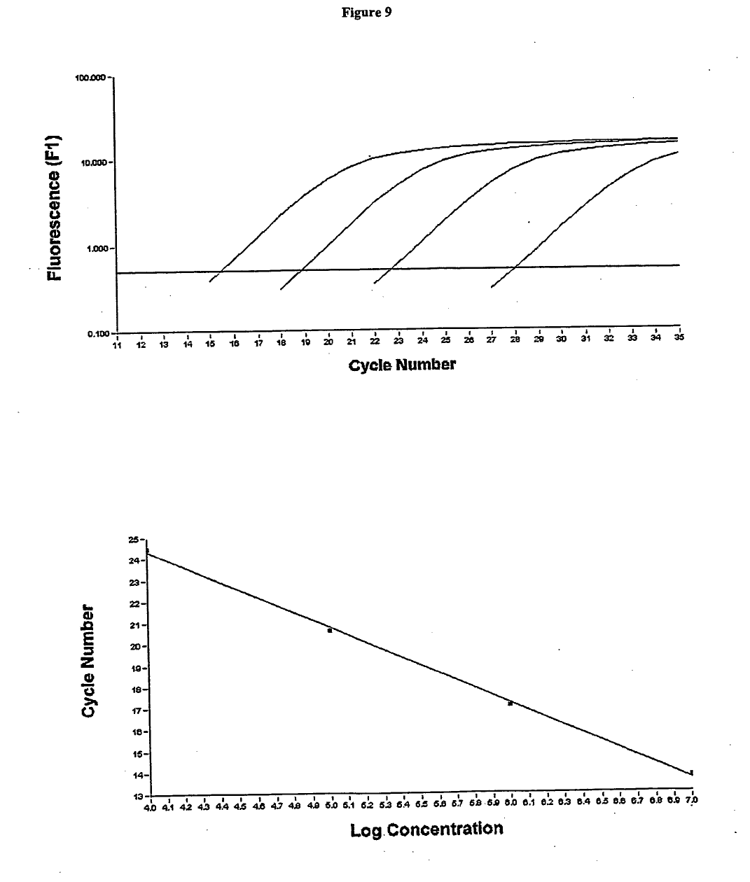

The full-length mRNA sequence of the KLK15 gene was amplified by PCR using gene-specific pruners, and the PCR product was cloned into a TOPO TA cloning vector (Invitrogen, Carlsbad, Calif., USA) according to manufacturer's instructions. Plasmids were purified using Mini-prep kit(Qiagen Inc., Valencia, Calif.). Different standard curves for actin and KLK15 were constructed using serial dilutions of the plasmid as described elsewhere (Bieche I, et al, Clin Chem 45:1148-1156, 199. These standards were included in each run. An example is given in FIG. 9. The reliability of the KLK15 assay was determined by evaluating within- and between-run precision. In all cases, the coefficients of variation were <10%.

PCR Amplification

The PCR reaction was carried out on the LightCycler™ system. For each run, a master mixture was prepared on ice, containing 1 μl of cDNA, 2 μl of LC DNA Master SYBR Green 1 mix, 50 ng of primers and 1.2 μl of 25 mM MgCl₂. The final volume was adjusted to 20 μl with water. After the reaction mixture was loaded into the glass capillary tube, the cycling conditions were carried out as follows: initial denaturation at 94° C. for 10 minutes, followed by 45 cycles of denaturation at 94° C. for 0 s, annealing at 63° C. for 5 s, and extension at 72° C. for 30 s. The temperature transition rate was set at 20° C. per second. Fluorescent product was measured by a single acquisition mode at 88° C. after each cycle. A melting curve was then performed by holding the temperature at 70° C. for 30 s followed by a gradual increase in temperature to 98° C. at a rate of 0.2° C./s, with the signal acquisition mode set at step. To verify the melting curve results, representative samples of the PCR products were purified and sequenced.

Statistical Analysis

First, an optimal cutoff value was defined by χ² analysis, based on the ability of KLK15 values to predict the PFS and OS of the study population. This cutoff (1.0 arbitrary units; 50^{th} percentile) identifies 50% of patients as being KLK15-positive.

Associations between clinicopathological parameters such as stage, grade, histotype, and residual tumor, and KLK15 expression were analyzed by the Chi-square test or the Fisher's Exact Test, when appropriate. For survival analysis, two different end points, cancer relapse (either local recurrence or distant metastasis) and death, were used to calculate progression-free and overall survival, respectively. Progression-free survival was defined as the time interval between the date of surgery and the date of identification of recurrent or metastatic disease. Overall survival was defined as the time interval between the date of surgery and the date of death.

The Cox univariate and multivariate proportional hazard regression model (Cox D R: R Stat Soc B 34:187-202, 1972) was used to evaluate the hazard ratio (relative risk of relapse or death in the KLK15-positive group). In the multivariate analysis, the models were adjusted for KLK15 expression, clinical stage, histologic grade, residual tumor and age.

Kaplan-Meier survival curves (Kaplan E L, Meier P., J Am Stat Assoc 53:457-481, 1958) were constructed for KLK15-positive and KLK15-negative patients. For further analysis, patients were divided into two groups either by the tumor grade (grade 1-2 vs. grade 3), tumor stage (stage I-II vs stage III-IV), or by the success of debulking (optimal vs. suboptimal debulking group). In each category, survival rates (disease-free survival and overall survival) were compared between KLK15-positive and KLK15-negative groups. The differences between the group survival curves were tested for statistical significance by the log rank test (Mantel N., Cancer Chemother Rep 50:163-170, 1966).

Results

KLK15 Expression in Benign and Cancerous Ovarian Tissues

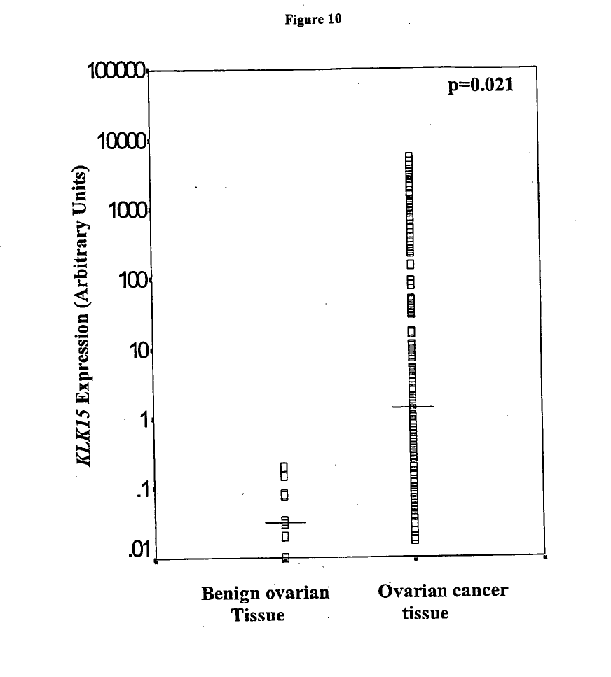

Table 5 shows the mean and median KLK15 expression levels in benign and malignant ovarian tumors. Expression levels were found to be much higher in cancerous tissues (mean=328 and median=1.0 arbitrary units) compared to benign ovarian tumors (mean=0.077 and median=0.056 arbitrary units). The distribution of KLK15 expression in ovarian cancer and benign tissues is depicted in FIG. 10. The differences between medians of the two groups were highly significant (p=0.021 by Mann-Whitney U test).

KLK15 Expression in Relation to Other Variables

As shown in Table 6, no significant associations were found between KLK15 expression and other clinical variables, except for a weakly significant difference between patients with optimal and suboptimal debulking.

Survival Analysis

Out of the 168 patients included in this study, follow-up information was available for 162 patients (median follow-up period 67 months), among whom 96 (59%) had relapsed and 61 (38%) died.

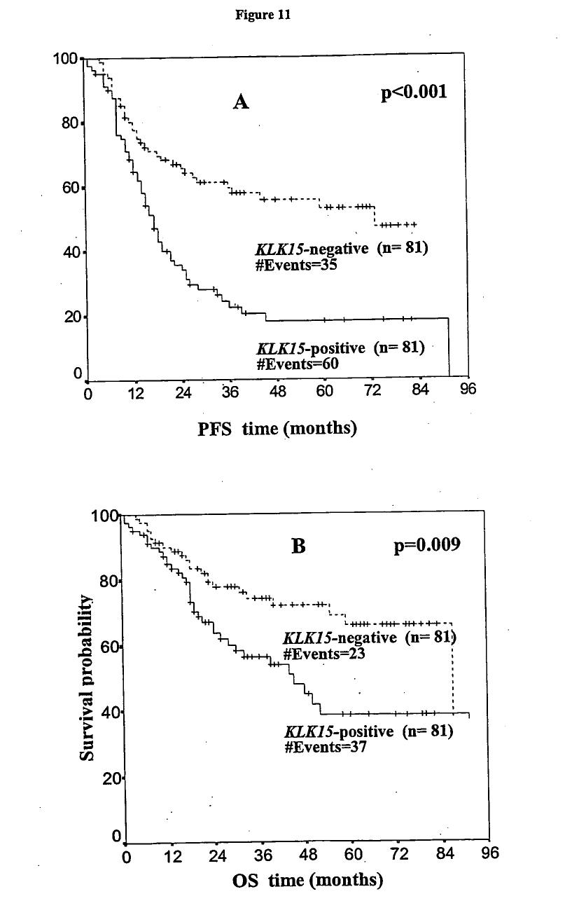

Kaplan-Meier survival curves demonstrated that patients with KLK15-positive tumors have substantially lower progression-free survival (PFS) (p<0.001) and overall survival (OS) (p=0.009) (FIG. 11), compared to those who are KLK15-negative. The strength of the associations between each individual prognostic factor and progression-free or overall survival are shown in the univariate analysis in Table 7. Stage of disease, histological grade and residual tumor size showed strong associations with cancer relapse and death (p<0.001). KLK15 expression was also found to be a significant predictor of lower PFS and OS (hazard ratio of 2.33 and 1.96, respectively and p value of <0.001 and 0.012, respectively).

When all the confounders were included in the Cox model (multivariate analysis, Table 7), only residual tumor size and grade, in addition to KLK15, retained their prognostic significance. KLK15 expression showed a hazard ratio of 2.27 and 1.79 and p value <0.001 and 0.039 for the PFS and OS, respectively. CA125, as a continuous variable, was found to be an unfavorable prognostic indicator in the Cox univariate analysis for PFS (p=0.007) but not for OS (p=0. 14). When KLK15 was included in the Cox model, CA125 did not retain its significance for PFS (p0.13 for CA125 and p=0.001 for KLK15) (data not shown).

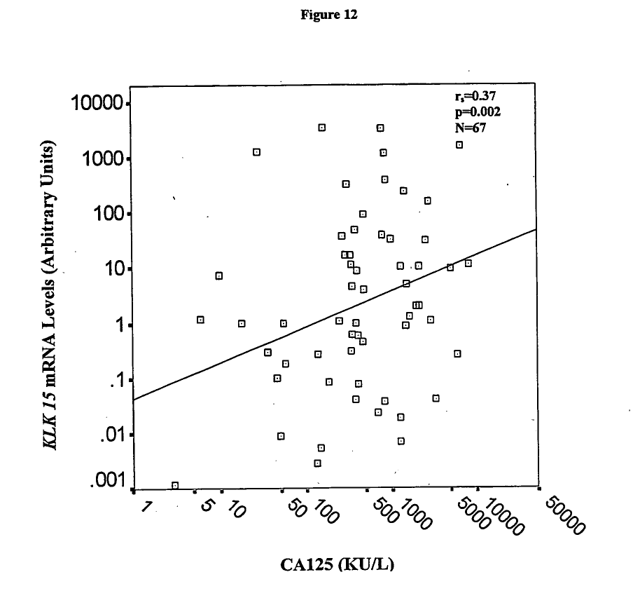

As shown in FIG. 12, a weak positive correlation was found-between the expression levels of KLK15 levels and pre-surgical serum CA125 (rₛ=0.37, p=0.002).

When Cox proportional hazard regression analysis was applied for subgroups of patients (Table 8), KLK15 was found to be a significant predictor of reduced PFS, but not OS, in the subgroups of patients with grade I-II (HR: 5.35, p=0.004), grade III (HR: 1.89, p=0.007), stage I-II (HR: 7.1, p=0.014), and stage III (HR: 1.93, p=0.004). KLK15 retained its prognostic significance after adjusting for other confounders (Table 8). KLK15 expression retained a highly statistically significant prognostic value for both PFS and OS in patients with optimal debulking, even after adjusting for all other confounders (Table 8).

Discussion

The results show that KLK15 is an independent marker of unfavorable prognosis in ovarian cancer.

KLK15 is a hormonally regulated gene (Yousef G M, et al; J Biol Chem 276:53-61, 2001). KLK15 is up-regulated mainly by androgens and to a lesser extent by progestins This regulation is possibly mediated through the androgen receptor (AR). Appreciable evidence implicates androgens in the pathogenesis of ovarian cancer (Risch H A; J Natl Cancer Inst 90:1774-1786, 1998), and supports the existence of a physiological interaction between androgens and the ovarian surface epithelium, as well as the possible role of this interaction in ovarian neoplasia (Levine D A, Boyd J; Cancer Res 61:908-911, 2001). Androgens stimulate growth of rodent ovarian epithelial cells in vivo, leading to benign ovarian neoplasms (Silva E G, et al; Mod Pathol 10:879-883, 1997). Furthermore, ovarian cancer patients have higher levels of circulating androgens prior to their diagnosis than women without cancer (Helzlsouer K J, et al; Jama 274:1926-1930, 1995). Additionally, the majority of ovarian cancers express AR (Chadha S, et al; Hum Pathol 24:90-95, 1993; and Kuhnel R, et al;. J Steroid Biochem 26:393-397, 1987), and ovarian cancer cell growth is inhibited in vitro by antiandrogens (Slotman B J, Rao B R; Cancer Lett 45:213-220, 1989). Recent observations show a correlation between AR and susceptibility to ovarian cancer (Kuhnel R, et al; J Steroid Biochem 26:393-397, 1987).

In this study, an optimal cutoff point equal to the 50th percentile was selected, based on the ability of KLK15 to predict progression-free and overall survival. The prognostic value of KLK15 is supported by the statistically significant differences between ovarian cancer and benign tissues between patients with optimal versus suboptimal debulking and by the positive correlation between the expression levels of KLK15 and pre-surgical serum CA125.

It is now widely accepted that no single biomarker will produce all the necessary information for diagnosis, prognosis and development of treatment strategies for patients with ovarian cancer. Instead, research is now focussing on generating a panel of ovarian cancer biomarkers. An artificial network approach for combining and interpreting information from a group of biomarkers will enable more accurate diagnosis and prognosis, a method that is currently underway and has already produced promising preliminary results (Zhang Z, et al; Gynecol Oncol 73:56-61, 1999; Woolas R P, et al; J Natl Cancer Inst 85:1748-1751, 1993; and Khan J, et al; Nat Med 7:673-679, 2001).

The results show a weak positive correlation between KLK15 expression and serum CA125 levels (FIG. 12). These results are consistent with previous reports showing that higher CA125 levels are associated with poor prognosis in ovarian cancer (de la Cuesta R, et al; Int J Biol Markers 14:106-114, 1999). On other hand, while high CA125 expression levels are associated only with the serous histological type (de la Cuesta R, et al; Int J Biol Markers 14:106-114, 1999), no significant relationships were found between KLK15 levels and any of the histological types of ovarian carcinoma (Table 6), implicating a possible role of KLK15 in monitoring non-serous ovarian cancer patients, where CA125 is not usually informative.

In conclusion, higher KLK15 expression was found to be an indicator of poor prognosis in ovarian cancer.

### EXAMPLE 3

Immunological reagents were generated for kallikrein 14, in order to develop an ELISA and immunohistochemical techniques to study its expression in normal and cancerous tissues and biological fluids. Recombinant hK14 was produced in P. pastoris, purified by affinity chromatography and injected into mice and rabbits for polyclonal antibody generation. Using the mouse and rabbit antisera, a sandwich-type immunofluorometric ELISA and immunohistochemical methodologies were developed for hK14. The ELISA was sensitive (detection limit of 0.1 μg/L), specific for hK14, linear from 0 to 20 μg/L with between-run and within-run CVs of <10%. hK14 was quantified in human tissue extracts and biological fluids. Highest levels were observed in the breast skin, prostate, seminal plasma and amniotic fluid, with almost undetectable levels in normal serum. hK14 concentration was higher in 40% of ovarian cancer tissues compared to normal ovarian tissues. Serum hK14 levels were elevated in a proportion of patients with ovarian (65%) and breast (40%) cancers. Immunohistochemical analyses indicated strong cytoplasmic staining of hK14 by the epithelial cells of normal and malignant skin, ovary, breast and testis.

Cloning of KLK14 into P. pastoris expression vector pPICZαA. KLK14 cDNA encoding the 227 amino acids of the mature form of the hK14 protein (corresponding to amino acids 25-251 of GenBank accession # AAK48524) (Yousef, G. M., et al. Cancer Res, 61: 3425-3431., 2001), was PCR amplified from vector construct pPICZαA-KLK14 (10 ng), previously produced by cloning amplified mature KLK14 cDNA into expression vector pPICZαA of the Easyselect™ Pichia pastoris yeast expression system (Invitrogen, Carlsbad, Calif.). The reaction was performed in a 50 μL reaction mixture containing Pfu DNA polymerase buffer [200 mM Tris-HCl (pH 8.8), 20 mM MgSO₄, 100 mM KCl, 100 mM (NH₄)₂SO₄, 1% Triton® X-100, 1 mg/ml nuclease-free BSA], 2 mM MgCl₂, 200 μM dNTPs, 100 ng of primer FP14-His (5′GAA GCT GAA TTC ATA ATT GGT GG 3′ [SEQ ID NO: 17]) and RP14-His (5′ TTT GTT CTA GAG CTT TGT CCC 3′ [SEQ ID NO: 18]), and 0.5 μL (1.25 U) of PfuTurbo DNA polymerase (Stratagene, La Jolla, Calif.), on an Eppendorf master cycler. The PCR cycling conditions were 95° C. for 1 min, followed by 95° C. for 30 s, 56° C. for 1 min, 72° C. for 1 min for 40 cycles, and a final extension at 72° C. for 7 min. Following PCR, amplified KLK14 was visualized with ethidium bromide on 2% agarose gels, extracted, digested with. EcoRI/XbaI and ligated into expression vector pPICZαA (Invitrogen) at corresponding restriction enzyme sites using standard techniques. Since the 5′ end of KLK14 insert was cloned in-frame with the yeast α-factor secretion signal and the 3′ end in-frame with C-terminal c-myc epitope and polyhistidine (His)₆ tags, the construct was denoted pPICZαA KLK14 ^{myc-His} and recombinant protein, hK14^{myc-His} The KLK14 sequence within the construct was confirmed with an automated DNA sequencer using vector-specific primers in both directions. Protein production. PmeI-linearized pPICZαA-KLK14^{myc-His}, as well as empty pPICZαA (negative control), were transformed into chemically competent P. Pastoris strain X-33 after which they were integrated into the yeast genome by homologous recombination. Transformed X-33 cells were then plated on YPDS (1% yeast extract, 2% peptone, 2% dextrose, 1 M sorbitol, 2% agar) plates containing Zeocin™, a selective reagent. A stable yeast transformant was selected as per the manufacturer's recommendations, inoculated in BMGY media [1 yeast extract, 2% peptone, 100 mM potassium phosphate (pH 6.0), 1.34% yeast nitrogen base, 40 mg/L biotin and 1% glycerol] overnight at 30° C. on a plate agitator at 250 rpm, diluted to OD₆₀₀=1.0 in BMMY (same as BMGY except that 1% glycerol is replaced with 0.5% methanol) and incubated under the same conditions as above for 6 days with a daily supplement of 1% methanol. The supernatant was collected by centrifugation at 4000-g for 20 minutes.

Protein purification. Recombinant hK14^{myc-His} was purified from the yeast culture supernatant by immobilized metal affinity chromatography (IMAC) using a Ni²⁺-nitriloacetic acid (NiNTA) column (Qiagen, Valencia, Calif.). Briefly, the yeast culture supernatant was diluted 4 times in equilibration buffer (50 mM Na₂HPO₄, 300 mM NaCl, 10 mM imidazole, pH 8.0) and loaded onto a column containing Ni-NTA resin previously equilibrated with the same buffer. The column was then washed twice with 5 volumes of equilibration buffer and the adsorbed hK14^{myc-His} eluted with a 20, 100, 250, 500, 1000 mM imidazole step gradient. All fractions were analyzed as described below and those containing hK14^{myc-His} were pooled and concentrated by ultrafiltration with an Amicon™ YM1O membrane (Millipore Corporation, Bedford, Mass.). The total protein concentration was subsequently determined using the Bradford bicinchoninic acid (BCA) method with bovine serum albumin as a standard (Pierce Chemical Co., Rockford, Ill.).

Detection of hK14^{myc-His.} To monitor recombinant hK14^{myc-His} production and purification, samples were subjected to sodium dodecyl sulfate polyacrylamide gel electrophoresis (SDS-PAGE) using the NuPAGE Bis-Tris electrophoresis system and 4-12% gradient polyacrylamide gels at 200V for 30 min (Invitrogen, Carlsbad, Calif.). Proteins were visualized with a Coomassie G-250 staining solution, SimplyBlue SafeStain (Invitrogen, Carlsbad, Calif.), according to the manufacturer's instructions. For western blot analysis, proteins were transferred onto a Hybond-C Extra nitrocellulose membrane (Amersham Biosciences, Piscataway, N.J.) at 30V for 1 hour, after separation by SDS-PAGE. The membrane was blocked with TBS-T [0.1 mol/L Tris-HCl buffer (pH 7.5) containing 0.15 mol/L NaCl and 0.1% Tween-20] supplemented with 5% non-fat dry milk, overnight at 4° C. Subsequently, the membrane was probed with a mouse anti-His (C-terminal) monoclonal antibody (Invitrogen, Carlsbad, Calif.) (diluted 1:5000 in TBS-T) for 1 hour at room temperature. After washing the membrane 3 times for 15 minutes with TBS-T, it was treated with horseradish peroxidase (HRP) conjugated goat anti-mouse (1:20,000 in TBS-T) (Amersham Biosciences, Piscataway, N.J.) for 1 hour at room temperature. Finally, the membrane was washed again as above and fluorescence was detected on X-Ray film using the SuperSignal® West Pico chemiluminescent substrate (Pierce Chemical Co., Rockford, Ill.).

Mass spectrometry. The identity of hK14^{myc-His} was confirmed by tandem mass spectrometry (MS-MS), as described previously in detail for recombinant hK10 (Luo, L. Y., et al, Clin Chem, 47: 237-246., 2001). N-terminal sequencing. N-terminal sequence analysis was performed to identify the amino acids at the N terminal end of recombinant hK14^{myc-His}. Recombinant hK14^{myc-His} (20 μg/lane) was first separated by SDS-PAGE and transferred onto a polyvinylidene difloride (PVDF) membrane (Amersham Biosciences, Piscataway, N.J.), previously immersed in 100% methanol, at 30V for 1 hour. After the transfer, the membrane was removed and rinsed with de-ionized water 3 times for 5 minutes prior to staining. Coomassie blue R-250 (0.1 solution in 40% methanol) was subsequently used to stain the membrane (5 min) followed by de-staining (5 min) in a 50% methanol solution. The membrane was then thoroughly washed with de-ionized water and air-dried. hK14^{myc-His} was subjected to automated N-terminal Edman degradation consisting of 5 cycles of Edman chemistry on a Porton/Beckman Gas-phase Microsequencer, followed by phenylthiohydantoin (PTH) analysis on an HPLC column.

Glycosylation status. hK14^{myc-His} was incubated with Peptide: N-glycosidase F (PNGaseF) (New England Biolabs, Beverly, Mass.), an amidase of 36 kDa which cleaves N-glycan chains from glycoproteins [between the innermost N-acetylglucosamine (GlcNAc) and Asn]. Briefly, 15 μg of purified hK14 was denatured in 2 μL of denaturing buffer (5% SDS, 10% β 3mercaptoethanol) at 100° C. for 5 min and immediately transferred to ice for an additional 5 min. One-tenth the volume of both G7 buffer [0.5 M sodium phosphate (pH 7.5)] and 10% NP-40 were then added, followed by 1 μl of PNGaseF. The reaction was incubated at 37° C. for 2 hours.

WO identical polyacrylamide gels containing 10 μg/lane of purified hK14^{myc-His} purified deglycosylated hK14^{myc-His} as well as horseradish peroxidase (a glycoprotein of ˜40 kDa; positive control) and soybean trypsin inhibitor (an unglycosylated protein of ˜21.5 kDa; negative control) were subjected to SDS-PAGE. One gel was stained with SimplyBlue SafeStain (Invitrogen, Carlsbad, Calif.) a Coomassie G-250 staining solution, and the other using the GelCode® Glycoprotein staining kit (Pierce Chemical Co., Rockford, Ill.). The latter allows for detection of glycoprotein sugar moieties on polyacrylamide gels. This gel was treated with periodic acid, which oxidizes the glycols present in glycoproteins to aldehydes, followed by immersion in the GelCode® Glycoprotein Stain, containing acidic fuchsin sulfite, the active agent.

Production of Antibodies Against hK14^{myc-His}

Purified recombinant hK14^{myc-His} (˜100 μg) was used as an immunogen and injected subcutaneously into Balb/C female mice and New Zealand white female rabbits for polyclonal antibody development. The protein was diluted 1:1 in complete Freund's adjuvant for the first injection and in incomplete Freund's adjuvant for subsequent injections. Injections were repeated three times for mice and six times for rabbits at 3-week intervals. Blood was drawn from the animals and tested for antibody generation every 2 weeks. To test for production of anti-hK14 polyclonal antibodies in mice and rabbits, the following immunoassay was used: sheep anti-mouse or goat anti-rabbit IgG (Jackson ImmunoResearch, West Grove, Pa.) was immobilized on 96-well opaque polystyrene plates. The mouse/rabbit serum was then applied to the plates at different dilutions ranging from 1:250 to 1:100000. After incubation (1 h) and washing, biotinylated recombinant hK14^{myc-His} was then added to each well (50 ng/well). Finally, after incubation (1 h) and washing, allaline phosphatase-conjugated streptavidin was added, incubated (15 min) and washed. Diflunisal phosphate (100 μL of a 1 mmol/L solution) in substrate buffer (0.1 mol/L Tris, pH 9.1, 0.1 mol/L NaCl, and 1 mmol/L MgCl₂) was added to each well and incubated for 10 min. Developing solution (100 μL, containing 1 mol/L Tris base, 0.4 mol/L NaOH, 2 mmol/L TbCl₃, and 3 mmol/L EDTA) was pipetted into each well and mixed for 1 min. The fluorescence was measured with a time-resolved fluorometer, the Cyberfluor 615 Immunoanalyzer (MDS Nordion, Kanata, ON). The calibration and data reduction were performed automatically, as described elsewhere (Christopoulos, T. K and Diamandis, E. P. Anal Chem, 64: 342-346., 1992, Hassapoglidou, S., et al Oncogene 8: 1501-1509., 1993).

ELISA for hK14

Standard Assay Procedure

A sandwich-type polyclonal (mouse/rabbit) ELISA was developed as follows: white polystyrene microtiter plates were coated with sheep anti-mouse IgG, Fc fragment-specific antibody (Jackson ImmunoResearch, West Grove, Pa.) by overnight incubation of 100 μL of coating antibody solution (containing 500 ng of antibody diluted in 50 mmol/L Tris buffer, pH 7.80) in each well. The plates were then washed three times with washing buffer (9 g/L NaCl and 0.5 g/L Tween 20 in 10 mmol L Tris buffer, pH 7.40). Mouse anti hK14 polyclonal antiserum was diluted 2000-fold in a general diluent [60 g/L bovine serum albumin, 50 mmol/L Tris (pH 7.8) and 0.5 g/L sodium azide], and 100 μL was applied to each well. After a 1 h incubation, the plates were washed six times with washing buffer.

hK14 calibrators or samples were then pipetted into each well (100 μL/well, diluted 1:1 in general diluent), incubated for 1 h with shaking; and then washed six times. Subsequently, 100 μL of rabbit anti-hK14 antiserum diluted 2000-fold in buffer A (containing the components of the general diluent plus 25 mL/L normal mouse serum, 100 mL/L normal goat serum, and 10 g/L bovine IgG) was applied to each well and incubated for 1 h, plates were then washed as described above. Finally, 100 μL/well of alkaline phosphatase-conjugated goat anti-rabbit IgG, Fc fragment-specific (Jackson ImmunoResearch, West Grove, Pa.), diluted 1000-fold in buffer A were added to each well, incubated for 30 min, and washed as above. Diflunisal phosphate in substrate buffer was added to each well and incubated for 10 min, followed by developing solution pipetted for 1 min. The fluorescence was measured with the Cyberfluor 615 Immunoanalyzer.

Determination of Sensitivity, Specificity and Linearity

Sensitivity. Recombinant hK14^{myc-His} was used to generate the calibration curve. hK14^{myc-His} calibrators were prepared by diluting purified recombinant hK14^{myc-His} in the general diluent. These calibrators were then used to define the detection limit of the assay.

Specificity. Recombinant hK14^{myc-His}, a biological fluid (seminal plasma) and a tissue extract (breast cancer cytosol), containing high hK14 levels, were used to determine the specificity of the developed immunoassay. These samples were first measured by the standard assay procedure described above. The mouse and rabbit anti-hK14 antisera were then successively replaced with sera from the same animals obtained before immunization (pre-immune sera). The samples were re-measured, and fluorescence counts were compared to those obtained by the standard assay. For further confirmation of the specificity of this assay, recombinant hK14^{myc-His} (1 μg, 100 ng and 20 ng) was subjected to western blot analysis using mouse and rabbit polyclonal pre-immune and immune anti-sera (all diluted 1:2000), separately, as primary antibodies.

In addition, the cross-reactivities of other homologous proteins were investigated using purified recombinant hK2-hK15 (produced in-house), all at a concentration of 1000 μg/L. Furthermore, hK14 (without c-myc and His epitopes; produced using similar techniques as hK14^{myc-His}) was also measured. Linearity. To determine the linearity of the hK14 immunoassay, serum, seminal plasma and breast cancer cytosol samples with high hK14 levels were serially diluted in the general diluent, and the amount of hK14 was measured using the standard assay procedure.

Preparation of Human Tissue Cytosolic Extracts and Biological Fluids

The presence of hK14 in normal human tissues (i.e. esophagus, tonsil, skin, testis, kidney, salivary gland, breast, fallopian tube, adrenal, bone, colon, endometrium, liver, lung, muscle, ovary, pancreas, pituitary, prostate, seminal vesicle, small intestine, spinal cord, spleen, stomach, thyroid, trachea and ureter), areas of the human brain (i.e. frontal cortex, cerebellum, hippocampus, medulla, midbrain, occipital cortex, pons, and temporal lobe) and cancerous breast and ovarian tissues was determined using the hK14 immunoassay. Cytosolic extracts were prepared as follows: various frozen human tissues (0.2 g) were pulverized on dry ice to fine powders. Extraction buffer (1 mL, containing 50 mmol/L Tris, pH 8.0, 150 mmol/L NaCl, 5 mmol/L EDTA, 10 g/L NP-40 surfactant, 1 mmol/L phenylmethylsulfonyl fluoride, 1 g/L aprotinin, 1 g/L leupeptin) was added to the tissue powders, and the mixture was incubated on ice for 30 min with repeated shaking and vortex-mixing every 10 min. Mixtures were then centrifuged at 14,000 g at 4° C. for 30 minutes. The supernatants were then collected. The levels of hK14 in ovarian and breast cancer cytosols were also determined. The biological fluids (seminal plasma, amniotic fluid, breast milk, cerebrospinal fluid, follicular fluid, serum, and ascites fluid from women with advanced ovarian cancer) screened were residual samples submitted for routine biochemical testing. All tissue cytosolic extracts and biological fluids were stored at ˜80° C. until use.

Recovery

Recombinant hK14^{myc-His} was added to the general diluent (control), normal serum (male and female), seminal plasma, amniotic fluid and breast cancer cytosols at different concentrations (5 and 10 μg/L) and measured with the hK14 immunoassay. Recoveries were then calculated after subtraction of the endogenous concentrations.

Immunohistochemistry

Immunohistochemical staining was performed according to a streptavidin-biotinperoxidase protocol using the DAKO LSABKit Peroxidase (DAKO; Glostrup, Denmark) and the hK14-specific rabbit polyclonal antibody raised against full-length recombinant hK14^{myc-His} protein produced in yeast, as the primary antibody. Briefly, 4 μm-thick paraffin tissue (including non-malignant and malignant breast, ovarian, testis and skin) sections were fixed in formalin, followed by deparaffinization in warm xylene for 5 min with two changes of xylene at room temperature (RT), and rehydration by transfer through graded alcohols. Endogenous peroxidase activity was blocked with 0.5% H₂0₂ in methanol for 10 min. The sections were then pretreated with 10 mmol/liter citrate buffer (pH 6.1) in a microwave oven for 5 min and incubated with the primary antibody (diluted 1:400) overnight at 4° C. After two washes of the sections in 50 mM Tris buffer (pH 7.6), the biotinylated link (DAKO; Carpinteria, Calif.) was applied for 15 min, followed by the streptavidin-peroxidase conjugate for another 15 min. The enzymatic reaction was developed in a freshly prepared solution of 3,3-diaminobenzidine tetrahydrochloride using DAKO Liquid DAB Substrate-Chromogen Solution for 10 min (brown color). The sections were then counter-stained with hemalum, dehydrated, cleared in xylene, and mounted. The staining pattern, the distribution of the immunostaining in each tissue, and the intensity of the staining were studied in detail.

Results

Recombinant hK14^{myc-His}

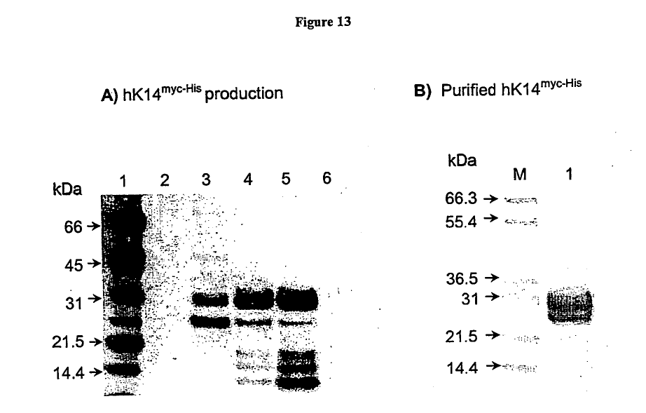

Recombinant hK14^{myc-His} was produced in the P. pastoris expression system, as a fusion protein consisting of the enzymatically active form of hK14 and C-terminal c-myc and His tags, with a predicted molecular mass of ˜28 kDa As detected by western blot analysis, the protein was expressed and secreted into the culture medium of a highly expressing X-33 P. pastoris clone in two forms, indicated by two distinct bands of ˜28 and 25 kDa, with the former being the predominant species (FIG. 13A; lanes 3-5). Furthermore, the protein appears to degrade after 4 days of methanol induction, as shown in FIG. 13 by the lower molecular weight bands (<21.5 kDa) in lanes 4-5. Recombinant hK14^{myc-His} was detected in the culture supernatant after 1 day of methanol induction (not shown) with highest levels after 6 days. hK14^{myc-His} was not observed in the supernatant of cells prior to induction (FIG. 13A; lane 2) or in the induced yeast cells transformed with the pPICZαA vector only (FIG. 13A; lane 6).

Recombinant hK14^{myc-His} was purified by IMAC after 6 days of methanol induction from the culture supernatant with 1 mg of purified hK14^{myc-His} per 250 mL of culture supernatant obtained, on average. As shown in FIG. 13B, purified hK14^{myc-His} is visualized as two bands of 28 and 25 kDa on a Coomassie blue stained SDS-PAGE gel. These bands were excised, digested with trypsin and sequenced by MS-MS. The m/z values of the tryptic peptides extracted on the nanoelectrospray mass spectrum (not shown) allowed for the calculation of their molecular masses. Confirmation of the sequence assignment was achieved using MS-MS on selected precursor ions. The partial sequences of these tryptic peptides were identified from the m/z spacing between adjacent fragments ions. For example, partially sequenced tryptic peptides, SSQPWQAALLAGPR {SEQ ID NO. 19] and AVRPEVTQACASPGTSCR [SEQ ID NO. 20], matched precisely with hK14 amino acid sequences 34-47 and 126-144, respectively (GenBank Accession # AAK48524).

The N-terminal sequence of the 28 kDa form of purified hK14^{myc-His} was Glu-Ala Glu-Phe-Ile-Ile (SEQ ID NO. 27). The first two amino acids identified, Glu-Ala, correspond to the last two amino acids of the yeast secretion α-factor. Although two potential cleavage sites exist for the removal of the yeast secretion α-factor, in this case, the dipeptidyl aminopeptidase involved in the maturation of α-factor (Ste 13 gene product), cleaved at the N-terminal side of Glu, resulting in only a partial removal of the α-factor. The other cleavage site is located at the C-terminal end of Ala, and if utilized, the Glu-Ala dipeptide would not have been incorporated at the N-terminus of hK14^{myc-His}. The next two amino acids, Glu-Phe, represent-the amino acids of the EcoRI restriction enzyme site, which were used to clone KLK14. The last two, Ile-Ile, match those of mature hK14, residues 25 and 26 in the protein sequence.

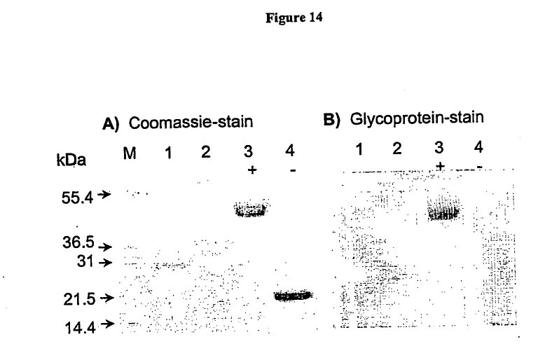

Given that two forms of hK14^{myc-His} were produced and that hK14 possesses a potential glycosylation site (Asn-Ile-Ser) in its primary sequence recognized by P. pastoris, it was originally hypothesized that the higher molecular weight species of 28 kDa was glycosylated. To determine this, purified hK14^{myc-His}, before and after incubation with PNGase-F, was separated by SDS-PAGE on two identical polyacrylamide gels, one stained by Coomassie-blue and the other with a glycoprotein stain (FIG. 14). Horseradish peroxidase, a glycoprotein, and soybean trypsin inhibitor, a non-glycosylated protein, were also included as positive and negative controls, respectively. On treatment with PNGase-F, the molecular weight of hK14^{myc-His} did not change (FIG. 14A). Furthermore, only the positive glycoprotein control was visualized on the gel stained for glycoproteins with acidic fuschin sulfite (FIG. 14B). Collectively, the results indicate that hK14^{myc-His} is not glycosylated. Since both versions of recombinant hK14^{myc-His} are unglycosylated, it is most probable that the 25 kDa version of hK14^{Myc-His} is a degraded species that arose via autocleavage or through cleavage by other proteases present in the yeast culture. These results, however, do not rule out the possibility that native hK14 may be glycosylated in vivo, similar to other native kallikreins including hK1 (Lu, H. S., et al, J. Int J Pept Protein Res, 33: 237-249, 1989.), hK2 (Mikolajczyk, S. D., et al, Eur J Biochem, 246: 440-446., 1997, Eerola, R., et al, Prostate, 31: 84-90, 1997) and hK3 (Belanger, A., et al, Prostate, 27: 187-197, 1995).

Characteristics of the hK14 ELISA

Configuration

The generation of polyclonal antibodies against recombinant hK14^{myc-His} was accomplished by injecting the recombinant proteins into mice and rabbits using standard techniques (Campbell, A. M. Production and Purification of Antibodies. In: E. P. Diamandis and T. K Christopoulos (eds.), Immunoassay, pp. 95-115. San Diego: Academic Press, 1996). The mouse and rabbit antisera demonstrated increasing hK14 immunoreactivity (higher titers) up to and including the 3rd booster injection. No immunoreactivity signficantly higher than background was noted when preimmune rabbit or mouse serum was substituted for the respective immune serum. Thus, the mouse and rabbit antibodies obtained after the 3rd booster injection were used to develop the hK14 immunofluorometric assay. A “sandwich-type” polyclonal immunoassay configuration, in which the capture antibody was generated in mice and the detection antibody in rabbits, was adopted. A secondary goat anti-rabbit polyclonal antibody, labeled with alkaline phosphatase, was also used, and the activity of alkaline phosphatase was measured by time resolved fluorometry (Christopoulos, T. K. and Diamandis, E. P. Anal Chem, 64: 342-346., 1992). This assay configuration does not necessitate any prior antibody purification and was previously found to be highly specific and sensitive for other kallikreins, including hK4 (Obiezu, C. et al, Clin Chem, 48: 1232-1240, 2002.), hK5, hK6 (Diamandis, E. P., et al, Clin Biochem, 33: 369-375, 2000.), hK8 (Kishi, T., et al, Clin Chem, 49: 87-96, 2003.), hK10 (Luo, L. Y. et al, Clin Chem, 47: 237-246., 2001.), hK11 (Diamandis, E. P., et al,. 62: 295-300., 2002) and hK13 (Kapadia, C., et al, Clin Chem, 49: 77-86, 2003.).

Sensitivity

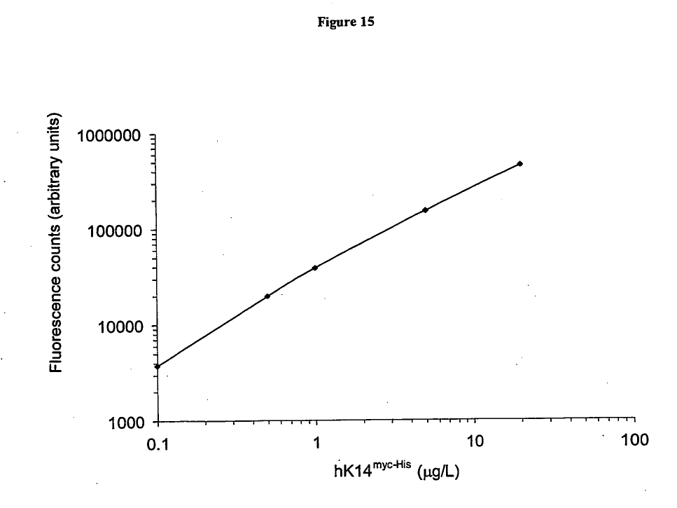

A typical calibration curve for the hK14 ELISA is shown in FIG. 15. Purified recombinant hK14^{myc-His}, diluted in 60 g/L BSA to 0.1, 0.5, 1, 5 and 20 μg/L were used as calibrators. Over this range, the assay showed a strong, linear relationship. The detection limit, defined as the concentration of hK14 that can be distinguished from zero with 95% confidence (mean +2 SD of zero calibrator), was 0.1 μg/L.

Specificity

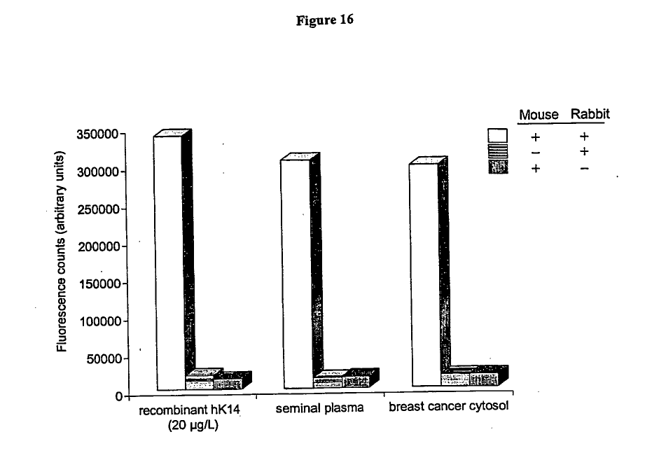

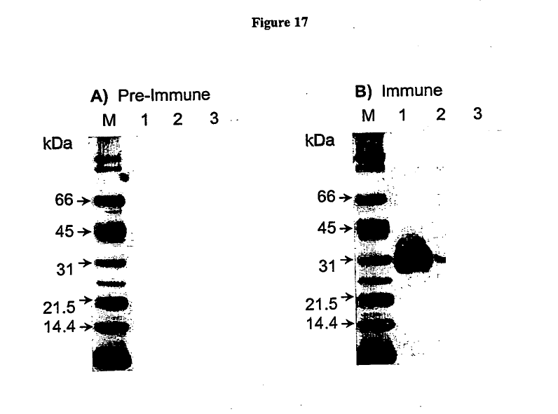

The specificity of the hK14 ELISA was confirmed by performing several experiments. First, immunoassay and western blots were performed using either preimmune or immune mouse and rabbit sera. When mouse and rabbit antisera was replaced with pre-immune mouse and rabbit sera in the immunoassay, fluorescence signals pertaining to recombinant hK14^{myc-His} (20 μg/L) and hK14-positive samples (seminal plasma and breast tissue extract) (˜350,000 arbitrary units) were reduced to background signals (˜16,000 arbitrary units) (FIG. 16). This experiment demonstrates that the fluorescence counts generated with the hK14 immunoassay represent the specific binding of mouse and rabbit anti-hK14 polyclonal antibodies to hK14. Second, western blot analysis of purified recombinant hK14^{myc-His} using rabbit pre-immune and immune sera produced bands corresponding to hK14^{myc-His} (1 μg and 100 ng) only when the membrane was probed with immune rabbit sera (FIG. 17). No bands representing hK14^{myc-His} were observed when the membranes were incubated with either pre-immune or immune mouse sera (data not shown). Third, the cross-reactivites of the polyclonal mouse and rabbit antibodies were assessed. Since hK14 is a member of the human kallikrein family, it shares significant amino acid similarity with other kallikreins, in particular to hK6 showing 48% amino acid identity. Thus, the cross-reactivities of recombinant hK2-hK15 were examined. All recombinant proteins produced readings comparable to background signals, even at concentrations 1000-fold higher than hK14. Furthermore, no cross-reactivity was observed when other His-tagged proteins (e.g. recombinant hK5^{myc-His}) were examined. In addition, recombinant hK14 (without c-myc/His epitopes) was tested and the established assay was able to detect hK14 with or without the c-myc/His epitopes equally well. In effect, this data confirms that the immunoassay measures hK14 with high specificity, efficiently discriminates hK14 from other similar proteins, and does not detect the polyhistidine tag.

Linearity and Precision

To assess the linearity of this assay, various samples (serum, seminal plasma and breast cancer cytosol) were serially diluted and hK14 was re-measured (data not shown). Good dilution linearity was observed with this assay, suggesting freedom from matrix effects. Within- and between-run precision was assessed with various hK14 calibrators and clinical samples. In all cases, the imprecision of the assay [coefficient of variation (CV)] was <10%.

Identification of hK14 in Human Tissue Extracts and Biological Fluids

Distribution of hK14 in Human Tissue Cytosolic Extracts

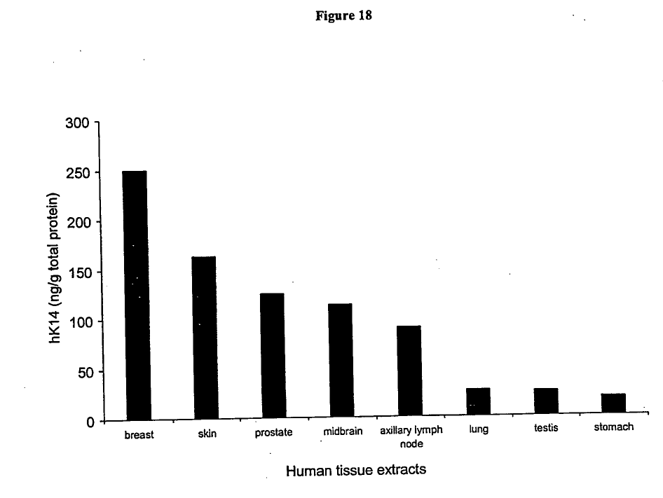

The levels of hK14 in various adult male and female tissues were quantified using the developed immunoassay. The data is presented graphically in FIG. 18. The amount of hK14 in these extracts was corrected for the total protein content and expressed as ng of hK14 per g of total protein. Highest hK14 levels were observed in the breast followed by skin, prostate, midbrain and axillary lymph nodes. Lower levels were seen in the lung, stomach and testis. No immunoreactivity was detected in the other tissues examined (See Materials and Methods section).

Immunohistochemical Localization of hK14

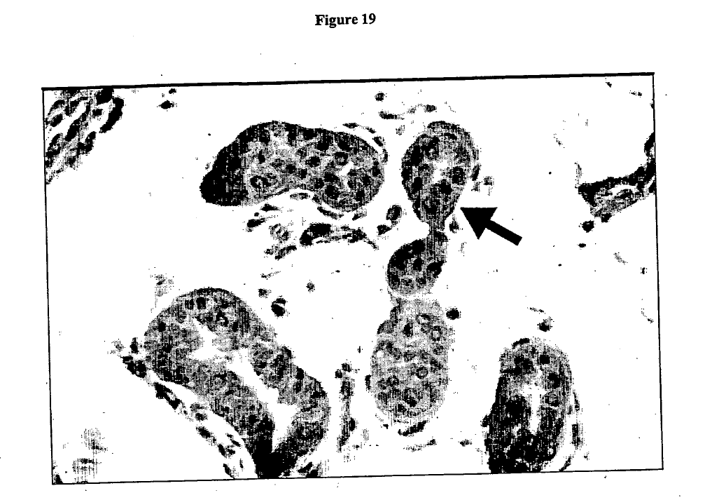

hK14 was immunohistochemically localized in the glandular epithelial cells derived from a variety of non-malignant and malignant tissues (FIG. 19). Strong immunostaining was generally observed in the cytoplasm of these epithelial cells, whereas stroma was typically negative.

hK14 in Biological Fluids

The concentration of hK14 in various biological fluids was quantified, as shown in Table 9. Highest levels of this kallikrein were seen in seminal plasma, followed by amniotic fluid and follicular fluid. Lower levels were obtained in male serum samples, while female serum, cerebrospinal fluid, ascites fluid and breast milk were all negative for hK14 (concentrations below the detection limit of 0.1 μg/L).

Recovery of hK14 From Biological Fluids

The recovery of hK14 in various biological fluids was incomplete and ranged from 24-60% in male serum, 18-35% in female serum, 30-64% in seminal plasma, 35-49% in amniotic fluid, and 40-56% in breast cancer cytosols.

Hormonal Regulation of hK14

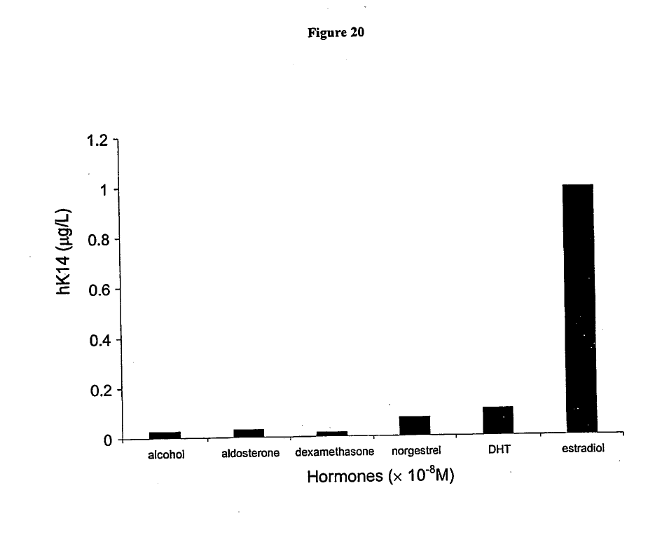

In order to study the hormonal regulation pattern of hK14, breast cancer cell line BT-474 was cultured, stimulated with various steroids at 10⁻⁸ mol/L final concentration and tissue culture supernatants were analyzed after 7 days incubation with the hK14 immunoassay. As illustrated in FIG. 20, the steroid that produced the most significant increase (38-fold) in hK14 concentration as compared with baseline hK14 levels (alcohol stimulation) was estradiol. DHT caused a 4-fold increase in hK14 levels, while norgestrel produced a 2.8-fold increase. These data suggest that KLK14 gene expression is mainly up-regulated by estrogens in the BT-474 breast cancer cell line.

hK14 Expression in Normal, Benign and Cancerous Ovarian Cytosols

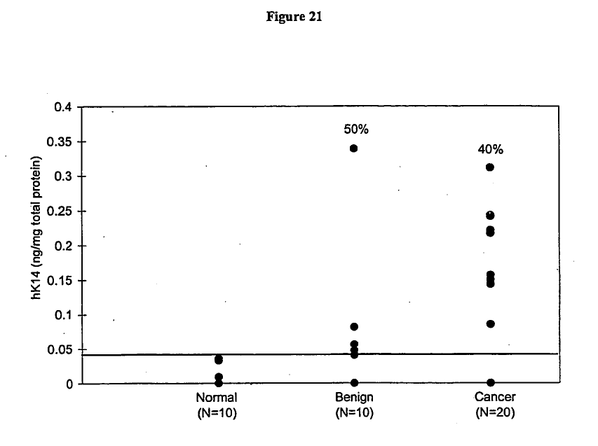

The levels of hK14 in 20 ovarian cancer tissue extracts, along with 10 normal ovarian tissues and 10 from patients with benign disease were quantified using the hK14 immunoassay. The hK14 values were corrected in terms of total protein and expressed as ng of hK14 per mg total protein. The results are displayed in FIG. 21. The levels of hK14 in normal ovarian tissue extracts did not exceed 0.03 ng/mg. Although 5 out of 10 extracts from the benign disease group surpassed this level, 4 of these values (with the exception of one extract measuring 0.33 ng/mg) remained below 0.08 ng/mg. Interestingly, 8 of 20 (40%) ovarian cancer tissue extracts contained even higher levels of hK14, all greater than 0.15 ng/mg, in comparison to that of normal and benign tissues (with that one exception). This data implies that in ovarian cancer patients, hK14 is generally over-expressed, in contrast to those with normal or benign disease.

hK14 in the Serum of Cancer Patients

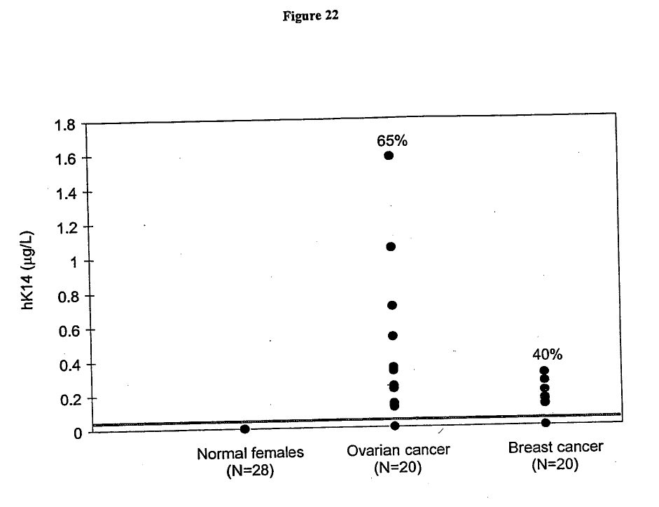

hK14 levels were analyzed in a total of 91 serum samples derived from patients with various malignancies, including ovarian (n=20), breast (n=20), prostate (n=31), testicular (n=10) and colon (n=10) cancers, along with 27 and 28 serum samples from healthy normal male and female subjects, respectively. hK14 was not detected in the serum of normal females, and reached a high level of 0.16 μg/L in healthy male sera examined (Table 9). Among patients with cancer (Table 10), 13 (65%) women with ovarian cancer and 8 (40%) with breast cancer demonstrated elevated levels of hK14, 0.12-1.58 μg/L and 0.12-0.3 μg/L, respectively (FIG. 22). Five individuals (16%) with prostate cancer displayed elevated hK14 levels (0.23-0.62 μg/L), whereas only 2 (20%) patients with colon cancer (0.18 and 0.26 μg/L), and 1 (10%) with testicular carcinoma (0.49 μg/L) possessed eminent hK14 concentrations.

Discussion

Extensive research throughout the past few decades has focused on the identification of tumor markers to aid in cancer screening, diagnosis, monitoring, prognosis and ultimately, to increase atient survival. Traditional and emerging tumor markers, either causally involved in carcinogenesis or incidental byproducts of malignant transformation, range from oncogenes, suppressor genes, cytokines, angiogenic factors, carbohydrate antigens and proteases, to cell-free nucleic acids, autoantibodies, adhesion proteins and circulating cancer cells (Chan, D. W. and Schwartz, M. K. Tumor Markers: Introduction and General Principles. In: E. P. Diamandis, H. A. Fritsch, H. Lilja, D. W. Chan, and M. H. Schwartz (eds.), Tumor Markers: Physiology, Pathobiology, Technology, and Clinical Applications, pp. 9-18. Washington, D.C.: AACC Press, 2002.). Proteases, in particular, have received a great deal of attention for their fundamental role in tumor progression and metastasis (Duffy, M. G. Clin. Exp. Metastasis, 10: 145-155, 1991., Noel, A., et al, Invasion Metastasis, 17: 221-239, 1997.).

Human tissue kallikreins are among proteases of the serine class, which have been implicated in carcinogenesis (Diamandis, E. P., et al, Trends Endocrinol Metab, 11: 54-60, 2000., Yousef, G. M. and Diamandis, E. P. Endocr Rev, 22: 184-204., 2001). This family of enzymes includes established (hK3/PSA) and prospective (hK2, hK5, hK6, hK8, hK10 and hK11) serological cancer biomarkers, as well as many potential prognostic/predictive indicators (Diamandis, E. P. and Yousef, G. M. Clin Chem, 48: 1198-1205, 2002.).

As described above, recombinant hK14^{myc-His} in the Picihia pastoris expression system was produced and purified. The protein was also administered as an immunogen to mice and rabbits for polyclonal antibody generation. The antibodies were used to develop a highly sensitive and specific ELISA suitable for hK14 quantification in biological fluids and tissue extracts and to perform immunohistochemical studies.

Using the ELISA, hK14 was measured in several biological fluids with the highest levels in seminal plasma and amniotic fluid (Table 9). This observation confirms that, in vivo, hK14 is a secreted protein. In contrast, the concentration of hK14 in the serum of healthy men and women and in follicular and ascites fluids was extremely low, close to the detection limit of the immunoassay (0.1 μg/L). The recovery of recombinant hK14^{myc-His} from biological fluids was also incomplete (ranging from 18-64%), a common finding among other kallikreins, including, hK3, hK6, hK8, hK10, hK11 and hK13 (Diamandis, E. P., et al, Cancer Res, 62: 295-300., 2002., Luo, L. Y., et al, Clin Chem, 47: 237-246., 2001., Diamandis, E. P., et al, Clin Biochem. 33: 369-375, 2000, Kishi, T., et al, Clin Chem, 49: 87-96, 2003, Kapadia, C., et al, Clin Chem, 49: 77-86, 2003., Yu, H. and Diamandis, E. P. Clin Chem, 39: 2108-2114, 1993.). One possible explanation for this phenomenon is that hK14 forms complexes with protease inhibitors, rendering them undetectable by immunoassay, and resulting in an underestimation of hK14 concentration (Zhang, W M et al, Clin Chem 44: 2471-2479, 1988). It has been well documented that other kallikreins, including hK2, hK3 and hK6, are sequestered (mainly in serum) by circulating protease inhibitors including α2-macroglobulin, protein C inhibitor, α1 antichymotrypsin α2-antiplasmin, α1-antitrypsin, anti-thrombin and protease inhibitor 6, forming complexes that are often not easily quantified (Christensson, A., et al, J. Biochem. 194: 755-763., 1990, Stenman, U. H., et al, Cancer Res, 51: 222-226, 1991, Lilja, H., et al, Clin Chem, 37: 1618-1625, 1991, Ferguson, R. A., et al, Clin Chem, 42: 675-684, 1996, Stephan, C., et al, Cancer Epidemiol Biomarkers Prev, 9: 1133-1147., 2000, Saedi, M. S., et al, Int J Cancer, 94: 558-563, 2001, Cao, Y., et al, Am. J. Pathol. 161: 2053-2063, 2002, Hutchinson, S., et al, Clin Chem, 49: 746-751, 2003). The free, as well as the bound forms of these kallikreins are useful biomarkers for the differential diagnosis of cancer (Stephan, C., et al, Cancer Epidemiol Biomarkers Prev, 9: 1133-1147., 2000, Rittenhouse, H. G., et al, Crit Rev Clin Lab Sci, 35: 275-368, 1998).

The tissue expression pattern of hK14 was determined by analyzing a panel of adult human tissue extracts with the ELISA. The protein was detectable in a small number of tissues, specifically the breast, slin, prostate, midbrain, axillary lymph nodes, lung, stomach and testis. Furthermore, as is the case for hK2, hK3, hK6, hK7, hK9, hK10, hK11 and hK13 (Diamandis, E. P., et al, Cancer Res. 62: 295-300., 2002, Luo, L. Y., et al, Clin Chem, 47: 237-246., 2001, Henttu, P. and Vihko, P. Ann Med, 26: 157-164, 1994, Petraki, C. D., et al, J Histochem Cytochem, 49: 1431-1441., 2001, Tanimoto, A, et al, Cancer, 86: 2074-2082, 1999, Yousef, G. et al, Cancer Res, 61: 7811-7818., 2001, Petrali, C. D., et al, J Histochem Cytocher, 50: 1247-1261., 2002, Petraki, C. D., et al, J Histochem Cytochem, 51: 493-501, 2003), hK14 was immunohistochemically localized in the cytoplasm of glandular epithelial cells from various tissues, both healthy and cancerous (FIG. 19), likely within the Golgi apparatus or secretory vesicles, further implying that this protein is secreted. These findings correlate well with each other and previous research on KLK14 mRNA expression by RT-PCR, indicating that KLK14 is transcribed in the brain, breast, prostate, and testis (Yousef, G. M., et al, Cancer Res, 61: 3425-3431., 2001).

Previous studies showed that KLK14 mRNA levels are highest in CNS tissues (i.e. brain, cerebellum and spinal cord) (Yousef, G. M., et al, Cancer Res, 61: 3425-3431., 2001). However, Hooper et al. reported restricted expression of KLK14 in the prostate, spleen and skeletal muscle (Hooper, J. D., et al, Genomics, 73: 117-122., 2001. The results described herein suggest that the hK14 protein is not detected in any CNS tissue, with the exception of the midbrain, in which relatively low levels were observed (FIG. 18). Furthermore, hK14 was undetectable in cerebrospinal fluid (Table 9). These data suggest that human kallikrein 14, at the protein level, is not significantly expressed in the CNS. The difference between KLK14 and hK14 levels in the CNS may be attributed to: 1) posttranslational regulation of the KLK14 gene; 2) efficient KLK14 transcription but rapid degradation of KLK14 mRNA (due to mRNA instability, short half-life); or 3) efficient translation of hK14 but rapid degradation shortly after synthesis.

Most, if not all, members of the kallikrein gene family are regulated by steroid hormones in the prostate, breast and ovarian cancer cell lines studied (Diamandis, E. P., et al, Trends Endocrinol Metab, 11: 54-60, 2000., Yousef, G. M. and Diamandis, E. P. Endocr Rev, 22: 184-204., 2001). Certain genes are predominately up-regulated by androgens and androgenic progestins (e.g. KLK2, KLK3, KLK4, KLK13 and KLK1 S), whereas others are primarily responsive to estrogens (e.g. KLK5, KLK6, KLK7, KLK9, KLK10 and KLK11) (Yousef, G. M. and Diamandis, E. P. Endocr Rev, 22: 184-204., 2001). Promoter/enhancer regions have only been characterized for KLK1, KLK2 and KLK3. With respect to the KLK14 gene, sequence analysis of its promoter region revealed the presence of a putative ARE and preliminary hormonal regulation studies indicate that KLK14 mRNA levels are predominately up-regulated by androgens in the breast (including BT-474 cells) and ovarian cancer cell lines tested. However, the immunofluorometric quantification of hK14 levels in the supernatant of the androgen and estrogen receptor-positive breast cancer cell line BT-474, after stimulation with various steroid hormones, indicated that the KLK14 gene is significantly up-regulated by estrogens, followed by androgens (DHT) and androgenic progestins (norgestrel). These results suggest that KLK14 is both androgen and estrogen responsive in the BT-474 cancer cell line.

These data are in accord with the tissue expression pattern of hK14 which shows relatively high levels of hK14 in the breast (an estrogenregulated tissue) and the prostate (an androgen-regulated tissue) from which it is likely secreted (Hooper, J. D., et al, Genomics, 73: 117-122., 2001), to form a constituent of seminal plasma, where it is also found at high levels.

ELISA was used to quantify hK14 in normal, benign and cancerous ovarian tissue extracts and in serum from normal individuals and patients with ovarian and breast cancer. Elevated hK14 levels were found in ovarian cancer tissue extracts (FIG. 22) and in the serum of a subset of ovarian and breast cancer patients, compared to normal (Table 10 and FIG. 21). The group of patients with elevated serum levels may also be those in whom tissue hK14 levels are overexpressed. Based on these findings, hK14 is an ovarian and breast cancer biomarker, in addition to its prognostic value at the mRNA level.

In addition to hK14, other kallikrein proteins, including hK5, hK6 (Diamandis, E. P., et al, J Clin Oncol, 21: 1035-1043, 2003, Hoffman, B. R, et al, Br J Cancer, 87: 763-771, 2002), hK8 (Kishi, T., et al, Cancer Res, 63: 2771-2774, 2003), hK10 (Luo, L. Y., et al, Cancer Res, 63: 807-811, 2003), hK11 (Diamandis, E. P., et al, Cancer Res 62: 295-300., 2002) and hF13 (Kapadia, C., et al, Clin Chem, 49: 77-86, 2003) are also elevated in the serum and/or tissues of ovarian cancer patients. Serum hK5 is also higher in a subgroup of breast cancer patients, while higher levels of hK3/PSA and hK10 are associated with a poor response to tamoxifen therapy (Foekens, J. A., et al, Br J Cancer, 79: 888-894, 1999, Luo, L. Y.,et al,. Br J Cancer, 86:-1790-1796, 2002). Given that these kallikreins are coexpressed and likely coordinately regulated, it is not unreasonable to speculate that they may form an enzymatic cascade pathway involved in ovarian and breast carcinogenesis by, as yet, unknown mechanisms (Yousef, G. M. and Diamandis, E. P. Minerva Endocrinol, 27: 157-166., 2002). hK14 may also be included in a panel with other ovarian and breast cancer biomarkers, including other kallikreins, to improve the diagnostic/prognostic potential for these lethal malignancies.

In conclusion, this is the first evidence to describe the development of specific reagents (recombinant hK14, polyclonal antibodies) and methodologies (ELISA and immunohistochemistry) for the quantitative and qualitative study of human kallikrein 14 at the protein level.

### EXAMPLE 4

Production of Recombinant Kallikrein 14 and Kallikrein 14^{myc-His} in P. pastoris

Cloning of KLK14 cDNA Into P. pastoris Expression Vector pPICZαA

Recombinant kallikrein 14 was produced in the P. pastoris yeast expression system. As suggested by hydrophobicity and structural homology analysis, kallikrein 14 is predicted to be a secreted trypsin-like serine protease of 251 amino acids (aa). The mature enzymatically active form of kallikrein 14 is thought to be comprised of 227 aa, corresponding to amino acids 25-251 (amino acids 1-18 constitute the signal peptide, and 18-24 the activation peptide) (GenBank accession # AAK48524) (Yousef et al, Cancer Res, 61:3425-3431, 2001). The cDNA encoding for this active form of kallikrein 14 was amplified by 2 sets of gene specific primers (FPL6/RPL6 and FP14-His/RP14-His), digested with EcoRI/XbaI and cloned into the P. pastoris expression vector, pPICZαA.

The constructs, pPICZαA-KLK14 and pPICZαA-KLK14^{myc-His} were engineered such that the secretion α-factor of Saccharontyces cerevisiae is in-frame with and directly upstream of the 5′ end of KLK14, and in the case of pPICZαA-KLK14^{myc-His}, that its 3′ end be in-frame with the C-terminal c-myc epitope and His tag of the pPICZαA plasmid. Thus, two versions of recombinant kallikrein 14 were produced, the non-tagged kallikrein 14 (predicted MW of 25 kDa) and the kallikrein 14^{myc-His}, containing C-terminal fusion tags (predicted MW of 28 kDa). Expression of recombinant kallikrein 14 and kallikrein 14^{myc-His} in P. pastoris.

Genotypically and phenotypically distinct P. pastoris yeast strains, X-33, GS115 and KM71H were transformed with PmeI-linearized constructs pPICZαA-KLK14 and pPICZαA-KLK14^{myc-His} as well as empty pPICZαA (control). The Mut phenotypes of 10 X-33 and GS115 colonies (transformed with either construct) were determined by examining their growth kinetics in the absence and presence of methanol.

The resultant phenotypes were all Mut⁺, indicating gene insertion or single crossover recombination at the AOX1 locus. The importance of assessing the Mut phenotype of X-33 and GS115 transformants stems from the fact that Mut⁺ and Mut^{S} strains exhibit distinct growth kinetics and hence require different growth strategies for protein production.

Recombinant kallikrein 14 and kallikrein 14^{myc-His} were detected in yeast culture supernatants as 25 kDa and 28 kDa proteins, respectively, in the majority (˜85%) of the 10 colonies examined from each transformed Pichia strain. Both proteins were identifiable after 1 day of methanol induction with highest levels obtained after 6 days. In general, the highest levels of kallikrein 14 and kallikrein 14^{myc-His} expression, as determined by comparing band intensities on stained gels or western blots, were obtained from recombinant X-33 strains. Optimal kallikrein 14 and kaolin 14^{myc-His} production in two X-33 (Mut⁺) clones was achieved by inducing expression with 1% methanol/day for a total of 6 days in buffered complex media (BMGY/BMMY).

Due to the weak sensitivity of the rabbit anti-kallikrein 14 polyclonal peptide antibodies, silver-stained polyacrylamide gels were used to monitor the production of recombinant kallikrein 14. Kallikrein 14 is only present in the supernatant of yeast cells induced by methanol indicated by a band of ˜28 kDa, and not in the supernatant prior to induction. With respect to recombinant kallikrein 14^{myc-His} production, western blot analysis using anti-His (C-term) antibody detected the presence of two forms of this protein in the yeast culture supernatant, as indicated by two distinct bands of ˜31 kDa and ˜28 kDa after methanol induction. Furthermore, the protein starts to degrade after 4 days of methanol induction by bands below the ˜21.5 kDa molecular weight marker. Importantly, kallikrein 14^{myc-His} was not observed in the supernatant of cells prior to induction or in the induced yeast cells transformed with the pPICZαA vector only.

Purification of Recombinant Kallikrein 14 Proteins

Recombinant kallikrein 14 was purified from the yeast culture supernatant of a highly expressing X-33 transformant by cation-exchange on an FPLC (Fast Performance Liquid Chromatography) system after 6 days of methanol induction. Since the pI of the recombinant kallikrein 14 protein was predicted to be 9.52, a cation exchange medium and an anionic buffer system with an operating pH below the pI of the protein, in this case pH 7.5, was chosen. This allowed the protein to bind to the negatively charged CM sepharose column, since it assumes a net positive charge at pH values lower than its pI. In general, chromatography systems, such as the FPLC system, typically result in higher resolution than batch techniques, which can lead to higher purity (Dunn B M et al, In: J. E. Colgan, B M et al (eds), Current Protocols in Protein Science, Vol. 1pp 8.2.1-8.2.30: John Wiley & Sons, Inc., 1997). Recombinant kallikrein 14 was eluted from the CM sepharose column at a KCl concentration range 0.3-0.4 M, with highest levels at 0.34 M. Elution fractions were combined and further concentrated 20 times. Purified kallikrein 14 is visualized as a single band of ˜25 kDa.

Recombinant kallikrein 14^{myc-His} was purified by IMAC after 6 days of methanol induction from the culture supematant of a highly expressing X-33 clone. Adsorption of Icallikrein 14^{myc-His} to the Ni-NTA matrix was performed at pH 8, such that the imidazole nitrogens in the histidyl residues of the His tag are in the nonprotonated form, allowing for their binding to the Ni²⁺ ions immobilized by the NTA groups. Elution was achieved by ligand exchange with imidazole. In general, the fusion His tag allows for high selectivity and efficiency, often providing a one-step purification of proteins at over 90% purity (Gaberc-porekar, V and Menart V, J Biochem Biophys Methods 49:335-360, 2001; Chaga, G S et al, J. Biochem Biophys Methods 49: 313-334, 2001). The recombinant kallikrein 14^{myc-His} protein was eluted from the Ni-NTA column at 20 and 100 mM imidazole concentrations. Afterward, these two eluates were combined and kalklirein 14^{myc-His} was further concentrated. Purified kallikrein 14^{myc-His} was visualized as two bands, close in molecular weight, after purification. Ultimately, 1 mg of purified kallikrein 14^{myc-His} was obtained per 250 mL of supernatant, on average.

Characterization of Recombinant kallikrein 14^{myc-His}

Mass Spectrometry

Identification of recombinant kallikrein 14 and kallikrein 14^{myc-His} by mass spectrometry confirmed that these proteins were, in fact, human kallikrein 14. In the case of recombinant kallikrein 14, sequenced tryptic peptides IITGGHTCTR (SEQ ID NO. 22), QVTHPNYNSR (SEQ ID NO. 23) and QACASPGTSCR (SEQ ID NO. 24) corresponded to amino acid sequences 25-33, 97-106 and 134-144, respectively, of the kallikrein 14 protein (GenBank Accession # AAK48524). Likewise, peptide fragments obtained from the kallikrein 14^{myc-His} sample, SSQPWQAALLAGPR (SEQ ID NO. 25) and AVRPIEVTQACASPGTSCR (SEQ ID NO. 26), matched with kallikrein 14 amino acid sequences 34-47 and 126-144, respectively.

N-Terminal Sequencing

The N-terminal sequence of purified recombinant kallikrein 14^{myc-His} was determined to be: Glu-Ala-Glu-Phe-Ile-Ile (SEQ ID NO.27),. The first two amino acids identified, Glu-Ala, correspond to the last two amino acids of the yeast secretion a-factor. Although two potential cleavage sites exist for the removal of the yeast secretion a-factor, in this case, the dipeptidyl aminopeptidase involved in the maturation of α-factor (Ste 13 gene product), cleaved at the N-terminal side of Glu, resulting in only a partial removal of the a-factor. The other cleavage site is located at the C-terminal end of Ala, and if utilized, the Glu-Ala dipeptide would not have been incorporated at the N-terminus of kallikrein 14^{myc-His}. The next two amino acids, Glu-Phe, represent the aa of the EcoRI restriction enzyme site which were used to clone KLK14. The last two, Ile-Ile, match the first two aa of mature kallikrein 14, residues 25 and 26 in the protein sequence.

Glycosylation Status

Given that kallikrein 14 possesses a potential glycosylation site (Asn-Ile-Ser) in its primary sequence recognized by P. pastoris, and the fact that two bands representing kallikrein 14^{myc-His} were visualized on western blots and coomassie-blue stained polyacrylamide gels, it was originally proposed that the higher molecular weight bands may correspond to a glycosylated version of kallikrein 14^{myc-His}. To determine this kallikrein 14^{myc-His} was subjected to in vitro deglycosylation by PNGase-F and separated by SDS-PAGE on two identical polyacrylamide gels, one stained by Coomassie-blue and the other with a glycoprotein stain. Horseradish peroxidase, a glycoprotein, and soybean trypsin inhibitor, a non-glycosylated protein, were also included as positive and negative controls, respectively. The results indicated that kallikrein 14^{myc-His} is not glycosylated. For one, in vitro deglycosylation did not generate a lower molecular weight version of kallikrein 14^{myc-His} as would be expected if sugar moieties present on kallikrein 14^{myc-His} were removed. Furthermore, bands representing kallikrein 14^{myc-His} were not visualized in the glycoprotein stained gel.

Enzymatic Activity

The fluorogenic synthetic peptides Boc-V-P-R-AMC (SEQ ID NO. 28) and Boc-A-A-P-F-AMC (SEQ ID NO. 29) were used to analyze the enzymatic activity of recombinant kallikrein 14 and kallikrein 14^{myc-His}. Given that kallikrein 14 is predicted to have a trypsin-like cleavage specificity, the use of Boc-V-P-R-AMC (a trypsin substrate) and Boc-A-A-P-F-AMC (a chymotrypsin substrate) also allowed for the experimental determination of kallikrein 14's substrate specificity.

Enzyme-mediated peptide hydrolysis is proportional to the arbitrary fluorescence counts obtained by the release of AMC from the peptide substrates. The results of the initial experiment indicate that recombinant kallikrein 14 is able to effectively cleave the trypsin substrate, V-P-R-AMC (SEQ ID NO. 30) at a similar efficiency as trypsin, the positive control. Fluorescence counts increased, in a dose-dependent manner, with increased concentrations of each enzyme. Thus, recombinant kallikrein 14 is enzymatically active. Conversely, kallikrein 14^{myc-His} was not able to hydrolyze V-P-R-AMC to a significant extent and is not considered to be active. In fact, fluorescence counts decreased to background counts comparable to those of the negative control (substrate only) when higher concentrations of this enzyme were employed.

When this experiment was repeated with the chymotrypsin substrate, A-A-P-F-AMC (SEQ ID NO. 31), neither recombinant kallikrein 14 nor kallikrein 14^{myc-His} hydrolyzed the peptide at any of the concentrations used, as indicated by background arbitrary fluorescence counts obtained, comparable to those of the negative control (substrate only). Chymotrypsin, as expected, effectively cleaved the peptide substrate in a dose-dependent fashion. This test experimentally confirms that kallikrein 14 possesses a trypsin-like, and not a chymotrypsin-like, substrate specificity, as previously predicted.

In the subsequent kinetic analysis, the enzymatic activity and substrate specificity of recombinant kallikrein 14 was further examined using trypsin substrate V-P-R-AMC (SEQ ID NO.30) along with trypsin (positive control) and chymotrypsin substrate A-A-P-F-AMC (SEQ ID NO.31) with chymotrypsin (positive control) and then measuring fluorescence counts at 30 sec intervals for a total of 15 min. With respect to V-P-R-AMC, similar results were obtained for both recombinant kallikrein 14 and trypsin at each concentration used. Fluorescence counts increased in a dose-dependent manner, consistent with the initial results described above. In addition, this experiment demonstrated that kallikrein 14- and trypsin-mediated hydrolysis of V-P-R-AMC increases over time. In the case of A-A-P-F-AMC (SEQ ID NO.31), only chymotrypsin, and not recombinant kallikrein 14, mediates hydrolysis of this peptide, as expected from the initial experiment. Chymotrypsin activity increases with increasing concentrations and over time, whereas kallikrein 14 produces only background fluorescence counts (identical to the negative control containing substrate only) at all concentrations throughout the experiment. These results further validate the finding that kallikrein 14 is a trypsin-like serine protease.

Characteristics of the Kallikrein 14 Immunofluorometric Assay

Configuration

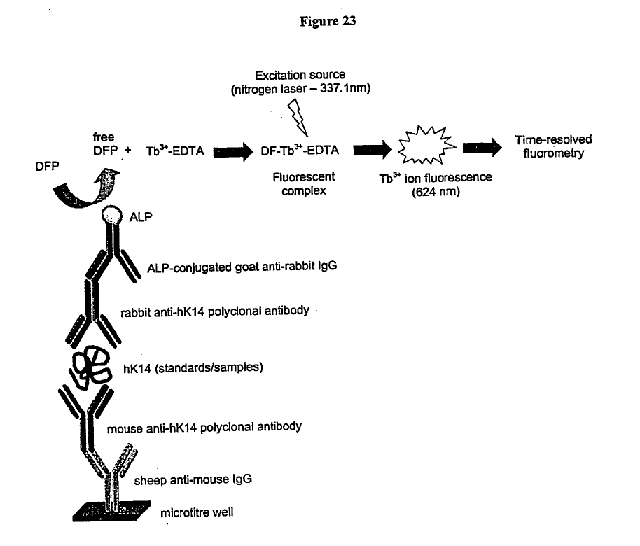

The generation of polyclonal antibodies against recombinant kallikrein 14^{myc-His} was accomplished by injecting the recombinant proteins into mice and rabbits using standard techniques (Campbell Production and Purification of Antibodies. In: E P Diamandis and T K Christopoulos (eds) Immunoassay, pp 95-115 San Diego: Academic Press, 1996). The mouse and rabbit antisera demonstrated increasing kallikrein 14 immunoreactivity (higher titers) up to and including the 3^{rd} booster injection, with no further increase after the 4^{th}, 5^{th} or 6^{th} injections. No immunoreactivity significantly higher than background (60 g/L BSA solution) was noted when preimmune rabbit or mouse serum was substituted for the respective immune serum. Thus, the mouse and rabbit antibodies obtained after the 3^{rd} booster injection were used to develop the kallikrein 14 immunofluorometric assay. A “sandwich-type” polyclonal immunoassay configuration, in which the capture antibody was generated in mice and the detection antibody in rabbits, was adopted (FIG. 23). A secondary goat anti-rabbit polyclonal antibody, labeled with alkaline phosphatase, was also used, and the activity of alkaline phosphatase was measured by time-resolved fluorometry (Christopoulos T K and Diamandis E P, Anal Chem 64:342-346, 1992). This assay configuration does not necessitate any prior antibody purification and was previously found to be highly specific and sensitive for other kallikreins, including hK6 (Diamandis E P et al, Clin Biochem 33:369-375, 2000), hK10 (Luo, L Y et al, Clin Chem, 47:237-246, 2001) and hK11 (Diamandis E P et al Cancer Res 62: 295-300, 2002).

Sensitivity

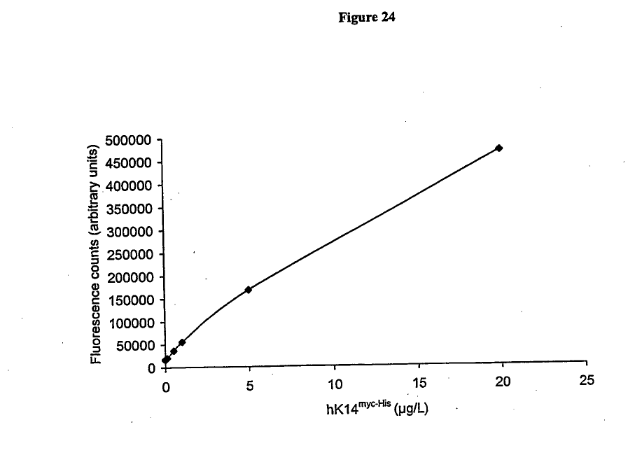

A typical calibration curve for the kallikrein 14 immunofluorometric assay is shown in FIG. 24. Purified recombinant kallikrein 14^{myc-His}, diluted in 60 g/L BSA to 0.1, 05, 1, 5 and 20 μg/L were used as calibrators. Over this range, the assay showed a strong, linear relationship. The detection limit, defined as the concentration of kallikrein 14 that can be distinguished from zero with 95% confidence (mean+2 SD of zero calibrator), was 0.1 μg/L.

Specificity

The specificity of the kallikrein 14 immunoassay was confirmed by performing several experiments. First, the immunoassay and western blot analyses were performed using both pre-immune and immune mouse and rabbit sera. The results obtained with the immunoassay indicated that when mouse and rabbit antisera was replaced with pre-immune mouse and rabbit sera, fluorescence signals of recombinant kallikrein 14^{myc-His} at 20 μg/L and kallikrein 14-positive samples (seminal plasma and breast tissue extract) (˜350,000 arbitrary units) were reduced to background signals (˜16,000 arbitrary units) (FIG. 16). This experiment demonstrates that the fluorescence counts generated with the kallikrein 14 immunoassay represent the specific binding of mouse and rabbit anti-kallikrein 14 polyclonal antibodies to kallikrein 14.

Western blot analysis of purified recombinant kallikrein 14^{myc-His} using mouse and rabbit pre-immune and immune sera produced bands corresponding to kallikrein 14^{myc-His} (1 μg and 100 ng) only when the membrane was probed with immune rabbit sera and not pre-immune rabbit sera. Also, no bands representing kallikrein 14^{myc-His} were observed when the membranes were incubated with either pre-immune or immune mouse sera (data not shown).

Second, the cross-reactivites of the polyclonal mouse and rabbit antibodies were assessed. Since kallikrein 14 is a member of the human kallikrein family, it shares significant amino acid similarity with other kallikreins, in particular to hK6 showing 48% aa identity. Thus, the cross-reactivities of recombinant hK2-hK15 were examined. All recombinant proteins produced readings comparable to background signals, even at concentrations 1000-fold higher than kallikrein 14. Furthermore, no cross-reactivity was observed when His-tagged proteins (recombinant hK5^{myc-His}) were examined. In addition, recombinant kallikrein 14 (without c-myc/His epitopes) was tested and the established assay was able to detect kallikrein 14 with or without the c-myc/His epitopes equally well. In effect, this data confirms that the kallikrein 14 immunoassay measures kallikrein 14 with high specificity, efficiently discriminates kallikrein 14 from other similar proteins, and does not detect the polyhistidine tag.

Linearity and Precision

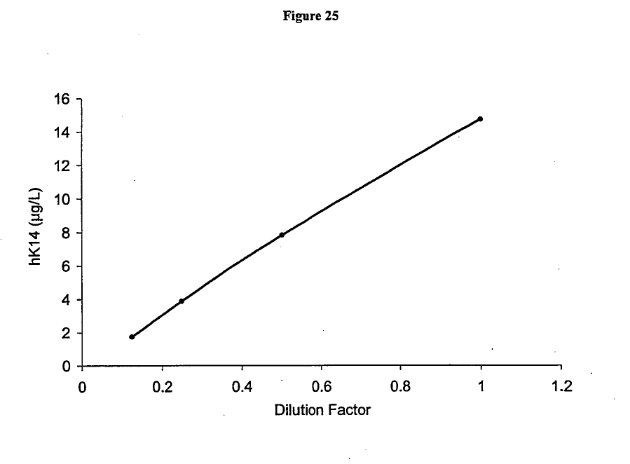

To assess the linearity of this assay, various samples (serum, seminal plasma and breast cancer cytosol) were serially diluted and kallikrein 14 was re-measured (FIG. 25). Good dilution linearity was observed with this assay, suggesting freedom from matrix effects. Within- and between-run precision was assessed with various kallikrein 14 calibrators and clinical samples. In all cases, the imprecision of the assay {coefficient of variation (CV)} was <10%.

Identification of Kallikrein 14 In Human Tissue Extracts and Biological Fluids

Distribution of Kallikrein 14 in Human Tissue Cytosolic Extracts

The levels of kallikrein 14 in various adult male and female tissues were quantified using the developed immunoassay. The data is presented graphically in FIG. 18. The amount of kallikrein 14 in these extracts was corrected for the total protein content and expressed as ng of kauliirein 14 per g of total protein. Highest kallikrein 14 levels were observed in the breast followed by skin, prostate, midbrain and axillary lymph nodes. Lower levels were seen in the lung, stomach and testis. No immunoreactivity was detected in the other tissues examined.

Kallikrein 14 in Biological Fluids

The concentration of kallikrein 14 in various biological fluids was quantified, as shown in Table 9. Highest levels of this kallikrein were seen in seminal plasma, followed by amniotic fluid and follicular fluid. Lower levels were obtained in male serum samples, while female serum, cerebrospinal fluid, ascites fluid and breast milk were all negative for kallikrein 14 (concentrations below the detection limit of 0.1 μg/L).

Recovery of Kallikrein 14 From Biological Fluids

The recovery of kallikrein 14 in various biological fluids was incomplete and ranged from 23.5-59.5% in male serum, 18-35% in female serum, 30.5-64% in seminal plasmna, 35-49.2% in amniotic fluid, and 40-55.5% in breast cancer cytosols.

Kallikrein 14 Expression in Breast Cancer Cytosols

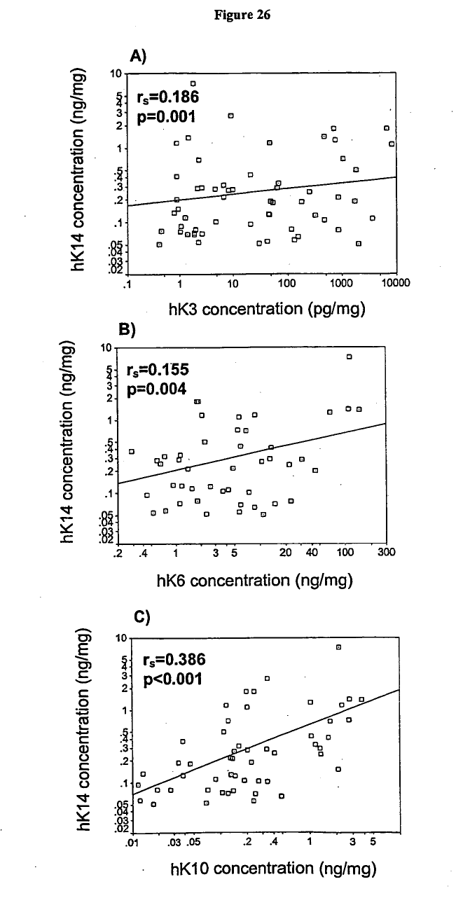

The expression levels of kallikhrein 14 in the tumor cytosols of 341 histologically confirmed breast cancer patients with ages ranging from 27 to 99 years (mean age 63.05 years) were determined using the developed immunoassay (Table 11). The mean level of kallikrein 14 expression was 0.18 ng/mg of total protein (expression levels ranged from 0.00 to 16.77 nglmg of total protein). A weakly positive correlation was observed between kallikrein 14 expression levels and those of other kallikreins including hK3 (rₛ 0.186 and p=0.001), hK6 (rₛ 0.155 and p=0.004) and hK10 (rₛ 0.386 and p<0.001) (FIG. 26). No correlations were found between kallikrein 14 expression levels and the ER (rₛ−0.08, p=0.14) or PR (rₛ−0.033, p=0.54). Kallikrein 14 expression levels did not show any statistically significant correlation with patient age.

When patients were stratified according to ER status, a statistically significant difference in kallikrein 14 expression levels was found between the ER-positive and ER-negative groups of patients (p=0.036), that is, a higher percentage of ER-negative patients displayed kallikrein 14-positive tumors (Table 12). Although a higher percentage of the PR-negative patients show kallikrein 14-positive tumors, the results were not statistically significant.

Kallikrein 14 Expression in Normal, Benign and Cancerous Ovarian Cytosols

The levels of kallikrein 14 in 20 ovarian cancer tissue extracts, along with 10 normal ovarian tissues and 10 from patients with benign disease were quantified using the kallikrein 14 immunoassay. The kallikrein 14 values were corrected in terms of total protein and expressed as ng of kallikrein 14 per mg total protein. The results are displayed in FIG. 21. The levels of kallikrein 14 in normal ovarian tissue extracts did not exceed 0.03 ng/mg. Although 5 out of 10 extracts from the benign disease group surpassed this level, 4 of these values (with the exception of one extract measuring 0.33 ng/mg) remained below 0.08 ng/mg. Interestingly, 8 of 20 (40%) ovarian cancer tissue extracts contained even higher levels of kallikrein 14, all greater than 0.15 ng/mg, in comparison to that of normal and benign tissues (with that one exception). This data implies that in ovarian cancer patients, the kallikrein 14 is generally over-expressed, in contrast to those with normal or benign disease.

Kallikrein 14\Kallikrein 14 in the Serum of Cancer Patients

Kallikrein 14 levels were analyzed in a total of 91 serum samples derived from patients with various malignancies, including ovarian (n=20), breast (n=20), prostate (n=31), testicular (n=10) and colon (n=10) cancers, along with 27 and 28 serum samples from healthy normal male and female subjects, respectively. Kallikrein 14 was not detected in the serum of normal females, and reached a high level of 0.16 μg/L in healthy male sera examined (Table 9). Among patients with cancer (Table 10), 13 (65%) women with ovarian cancer and 8 (40%) with breast cancer demonstrated elevated levels of kallikrein 14, 0.12-1.58 μg/L and 0.12-0.3 μg/L, respectively (FIG. 22). Five individuals (16%) with prostate cancer displayed elevated kallikrein 14 levels (0.23-0.62 μg/L), whereas only 2 (20%) patients with colon cancer (0.18 and 0.26 μg/L), and 1 (10%) with testicular carcinoma (0.49 μg/L) possessed eminent kallikrein 14 concentrations.

Hormonal Regulation of Kallikrein 14

In order to study the hormonal regulation pattern of kallikrein 14, the breast cancer cell line BT-474 was cultured, stimulated with various steroids at 10⁻⁸ mol/L inal concentration and tissue culture supernatants were analyzed after 7 days incubation with the kallikrein 14 immunoassay. As illustrated in FIG. 20, the steroid that produced the most significant increase (38-fold) in kallikrein 14 concentration as compared with baseline kallikrein 14 levels (alcohol stimulation) was estradiol. DHT caused a 4-fold increase in kallikrein 14 levels, while norgestrel produced a 2.8-fold increase. These data suggest that KLK14 gene expression is mainly up-regulated by estrogens in the BT-474 breast cancer cell line.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Below full citations are set out for the references referred to in the specification.

TABLE 1
Experimental protocol used for quantitative
PCR amplification of the KLK14 gene

| Segment | Temperature | Hold Time | Slope | Application |
|---|---|---|---|---|
| Number | Target (° C.) | (sec) | (° C./sec) | Mode |
| Program: | Denaturation | | Cycles: | 1 |
| 1 | 95 | 600 | 20 | None |
| Program: | PCR | | Cycles: | 35 |
| 1 | 95 | 0 | 20 | None |
| 2 | 62 | 5 | 20 | None |
| 3 | 72 | 40 | 20 | None |
| 4 | 85 | 5 | 20 | Single |
| Program: | Melting | | Cycles: | 1 |
| 1 | 95 | 0 | 20 | None |
| 2 | 72 | 30 | 20 | None |
| 3 | 97 | 0 | 0.2 | Step |
| Program: | Cooling | | Cycles: | 1 |
| 1 | 40 | 30 | 1 | None |

TABLE 2
Descriptive statistics of KLK14 expression
in cancer, benign and non-cancer tissues.

KLK14

| expression¹ | | Percentiles | | | | |
|---|---|---|---|---|---|---|
| (arbitrary | | | | 50 | | |
| units) | Range | 10 | 25 | (Median) | 75 | 90 |
| Cancer Tissues | | | | | | |
| (N = 155) | 0.01-97,643 | 0.011 | 0.06 | 1.00 | 22 | 128 |
| Benign Tissues | | | | | | |
| (N = 10) | 0.17-5,000 | 0.32 | 1.75 | 6.5 | 98 | 4,510 |
| Non Cancer | | | | | | |
| Tissues | | | | | | |
| (N = 10) | 3.2-11,000 | 3.9 | 40 | 74 | 298 | 9,913 |

¹N = number of tissues.

TABLE 3
Relationship between KLK14 status and other
variables in 155 ovarian cancer patients.

| | | No. of patients (%) | | |
|---|---|---|---|---|
| | | KLK14- | KLK14- | |
| Variable | Patients | negative | positive | p value |
| Stage | | | | |
| I/II | 45 | 16 (35.6) | 29 (64.4) | 0.020^{a} |
| III/IV | 103 | 59 (57.3) | 44 (42.7) | |
| x | 7 | | | |
| Grade | | | | |
| G1/G2 | 53 | 23 (43.4) | 30 (56.6) | 0.23^{a} |
| G3 | 95 | 52 (54.7) | 43 (45.3) | |
| x | 7 | | | |
| Histotype | | | | |
| Serous | 70 | 39 (55.7) | 31 (44.3) | |
| Endometrioid | 28 | 9 (32.0) | 19 (68.0) | |
| Mucinous | 15 | 10 (66.7) | 5 (33.3) | 0.30^{b} |
| Clear Cell | 16 | 10 (62.5) | 6 (37.7) | |
| Undiffer- | 24 | 11 (45.8) | 13 (54.2) | |
| entiated | | | | |
| x | 2 | | | |
| Residual | | | | |
| tumor (cm) | | | | |
| 0 | 67 | 28 (41.8) | 39 (58.2) | |
| 1-2 | 27 | 17 (63.0) | 10 (37.0) | 0.11^{b} |
| >2 | 53 | 30 (56.6) | 23 (43.4) | |
| x | 8 | | | |
| Debulking | | | | |
| success^{c} | | | | |
| OD | 79 | 33 (41.8) | 46 (58.2) | 0.020^{a} |
| SO | 68 | 42 (61.8) | 26 (38.2) | |
| x | 8 | | | |
| Menopause | | | | |
| Pre/peri | 48 | 21 (43.8) | 27 (56.3) | 0.29^{a} |
| Post | 105 | 57 (54.3) | 48 (45.72) | |
| x | 2 | | | |
| Response | | | | |
| to CTX^{d} | | | | |
| CR/PR | 125 | 58 (46.4) | 67 (53.6) | 0.008^{a} |
| NC/PD | 17 | 14 (82.4) | 3 (17.6) | |
| NE | 13 | | | |

^{a}Fisher's Exact Test
^{b}χ² test.
^{c}OD; Optimal debulking (0-1 cm), SO; Suboptimal debulking (>1 cm)
^{d}CTX; chemotherapy, NC; no change, PD; progressive disease, CR; complete
response, PR; partial response, NE; not evaluated.
x Status unknown.

TABLE 4
Univariate and Multivariate Analysis of KLK14 with Regard to PFS and OS

| | Progression-free survival | | | Overall survival | | |
|---|---|---|---|---|---|---|
| Variable | HR^{a} | 95% CI^{b} | p value | HR^{a} | 95% CI^{b} | p value |

Univariate analysis

| | | | | | | |
|---|---|---|---|---|---|---|
| KLK14 (N = 147) | | | | | | |
| negative | 1.00 | | | 1.00 | | |
| positive | 0.38 | 0.24-0.62 | <0.001 | 0.33 | 0.18-0.61 | <0.001 |
| as a continuous variable | 0.99 | 0.99-1.00 | 0.48 | 0.99 | 0.99-1.00 | 0.78 |
| Stage of disease (ordinal) | 2.48 | 1.88-3.25 | <0.001 | 2.51 | 1.76-3.57 | <0.001 |
| Grading (ordinal) | 2.068 | 1.57-2.72 | <0.001 | 2.13 | 1.46-3.09 | <0.001 |
| Residual tumor (ordinal) | 1.27 | 1.21-1.33 | <0.001 | 1.30 | 1.21-1.39 | <0.001 |
| Histologic type^{c} | 1.48 | 1.00-2.18 | 0.046 | 1.29 | 0.79-2.11 | 0.30 |
| Age | 1.01 | 0.99-1.03 | 0.12 | 1.01 | 0.99-1.03 | 0.13 |

Multivariate analysis.

| | | | | | | |
|---|---|---|---|---|---|---|
| KLK14 (N = 144) | | | | | | |
| Negative | 1.00 | | | 1.00 | | |
| Positive | 0.53 | 0.31-0.93 | 0.027 | 0.42 | 0.21-0.84 | 0.014 |
| as a continuous variable | 0.99 | 0.98-1.00 | 0.15 | 0.99 | 0.98-1.01 | 0.20 |
| Stage of disease (ordinal) | 1.38 | 0.89-2.14 | 0.14 | 1.57 | 0.91-2.73 | 0.11 |
| Grading (ordinal) | 1.61 | 1.02-2.56 | 0.041 | 1.35 | 0.75-2.41 | 0.30 |
| Residual tumor (ordinal) | 1.21 | 1.09-1.33 | <0.001 | 1.31 | 1.16-1.48 | <0.001 |
| Histologic type^{c} | 1.04 | 0.86-1.25 | 0.65 | 1.26 | 1.01-1.58 | 0.035 |
| Age | 1.02 | 0.99-1.04 | 0.15 | 1.02 | 0.99-1.05 | 0.13 |

^{a}Hazard ratio (HR) estimated from Cox proportional hazard regression model
^{b}Confidence interval of the estimated HR.
^{c}Serous vs. others

TABLE 5
Descriptive Statistics of KLK15 expression in Cancer and Benign tissues.

| | | | Percentiles | | | | |
|---|---|---|---|---|---|---|---|
| KLK15 expression | | | | | 50 | | |
| (arbitrary units) | Mean ± SE^{a} | Range | 10 | 25 | (Median) | 75 | 90 |
| Cancer Tissues | | | | | | | |
| (N = 168) | 328 ± 64 | 0.00-5330 | 0.00 | 0.025 | 1.00 | 38 | 1179 |
| Benign Tissues | | | | | | | |
| (N = 10) | 0.08 ± 0.04 | 0.00-0.20 | 0.00 | 0.008 | 0.056 | 0.16 | 0.19 |

^{a}Standard error

TABLE 6
Relationship between KLK15 status and other
variables in 168 ovarian cancer patients

No. of patients (%)

| | | KLK15 | KLK15 | |
|---|---|---|---|---|
| Variable | Patients | negative¹ | positive | p value |
| Stage | | | | |
| I/II | 42 | 22 (52.4) | 20 (47.6) | 0.72^{a} |
| III | 120 | 58 (48.3) | 62 (51.7) | |
| X | 6 | | | |
| Grade | | | | |
| G1/G2 | 53 | 29 (53.7) | 25 (46.3) | 0.51^{a} |
| G3 | 95 | 51 (47.2) | 57 (52.8) | |
| X | 7 | | | |
| Histotype | | | | |
| Serous | 76 | 38 (50.0) | 38 (50.0) | |
| Endometrioid | 28 | 12 (42.9) | 16 (57.1) | |
| Mucinous | 17 | 7 (41.2) | 10 (58.8) | 0.57^{b} |
| Clear cell | 16 | 6 (40.0) | 9 (60.0) | |
| Undiffer- | 28 | 17 (60.7) | 11 (39.3) | |
| entiated | | | | |
| x | 3 | | | |
| Residual | | | | |
| tumor (cm) | | | | |
| 0 | 68 | 38 (55.9) | 30 (44.1) | |
| 1-2 | 28 | 12 (42.9) | 16 (57.1) | 0.31^{b} |
| >2 | 66 | 29 (43.9) | 37 (56.1) | |
| x | 6 | | | |
| Debulking | | | | |
| success^{c} | | | | |
| OD | 82 | 33 (40.2) | 49 (59.8) | 0.028^{a} |
| SO | 80 | 46 (57.5) | 34 (42.5) | |
| X | 6 | | | |
| Menopause | | | | |
| Pre/peri | 47 | 26 (45.6) | 31 (54.4) | 0.26^{a} |
| Post | 111 | 58 (52.2) | 53 (47.8) | |
| Response | | | | |
| to CTX^{d} | | | | |
| NC/PD | 17 | 5 (29.4) | 12 (70.6) | 0.076^{a} |
| CR/PR | 141 | 75 (53.2) | 66 (46.8) | |
| NE | 10 | | | |

¹Cutoff was equal to 50^{th} percentile
^{a}Fisher's Exact Test
^{b}χ² test.
^{c}OD; Optimal debulking (0-1 cm), SO; Suboptimal debulking (>1 cm)
^{d}CTX; chemotherapy, NC; no change, PD; progressive disease, CR; complete
response, PR; partial response, NE; not evaluated.
x Status unknown.

TABLE 7
Univariate and Multivariate Analysis of KLK15 with
Regard to Progression-Free and Overall Survival.

| | Progression-free survival | | | Overall survival | | |
|---|---|---|---|---|---|---|
| Variable | HR^{a} | 95% CI^{b} | p value | HR^{a} | 95% CI^{b} | p value |

Univariate analysis.

| | | | | | | |
|---|---|---|---|---|---|---|
| KLK15 (N = 161) | | | | | | |
| Negative | 1.00 | | | 1.00 | | |
| Positive | 2.33 | 1.52-3.55 | <0.001 | 1.96 | 1.16-3.31 | 0.012 |
| As a continuous variable | 1.001 | 0.99-1.003 | 0.24 | 1.002 | 0.99-1.004 | 0.16 |
| Stage of disease (ordinal) | 2.48 | 1.89-3.25 | <0.001 | 2.52 | 1.76-3.58 | <0.001 |
| Grading (ordinal) | 2.09 | 1.58-2.75 | <0.001 | 2.14 | 1.47-3.11 | <0.001 |
| Residual tumor (ordinal) | 1.27 | 1.21-1.33 | <0.001 | 1.30 | 1.21-1.40 | <0.001 |
| Histologic type^{c} | 1.46 | 0.99-2.14 | 0.055 | 1.27 | 0.78-2.07 | 0.32 |
| Age | 1.01 | 0.99-1.03 | 0.12 | 1.01 | 0.99-1.03 | 0.11 |

Multivariate analysis.

| | | | | | | |
|---|---|---|---|---|---|---|
| KLK15 (N = 144) | | | | | | |
| Negative | 1.00 | | | 1.00 | | |
| Positive | 2.27 | 1.46-3.54 | <0.001 | 1.79 | 1.03-3.13 | 0.039 |
| Stage of disease (ordinal) | 1.35 | 0.95-1.91 | 0.087 | 1.39 | 0.87-2.22 | 0.16 |
| Grading (ordinal) | 2.03 | 1.35-3.05 | <0.001 | 1.92 | 1.07-3.43 | 0.027 |
| Residual tumor (ordinal) | 1.16 | 1.08-1.25 | <0.001 | 1.21 | 1.10-1.33 | <0.001 |
| Histologic type^{c} | 1.03 | 0.67-1.58 | 0.88 | 1.38 | 0.80-2.38 | 0.24 |
| Age | 1.02 | 0.99-1.05 | 0.087 | 1.03 | 1.00-1.05 | 0.044 |

^{a}Hazard ratio (HR) estimated from Cox proportional hazard regression model
^{b}Confidence interval of the estimated HR.
^{c}Serous vs. others

TABLE 8
Cox proportional hazard regression analysis for subgroups of patients

| | Progression-free survival | | | Overall survival | | |
|---|---|---|---|---|---|---|
| Variable | HR^{a} | 95% CI^{b} | p value | HR^{a} | 95% CI^{b} | p value |
| Tumor grade I-II | | | | | | |
| KLK15 unadjusted | 5.35 | 1.73-16.55 | 0.004 | 2.67 | 0.66-10.73 | 0.16 |
| KLK15 adjusted^{c} | 4.12 | 1.16-14.55 | 0.027 | 1.84 | 0.38-8.95 | 0.44 |
| Tumor grade III | | | | | | |
| KLK15 unadjusted | 1.89 | 1.18-3.01 | 0.007 | 1.68 | 0.95-2.99 | 0.073 |
| KLK15 adjusted^{c} | 2.29 | 1.39-3.75 | 0.001 | 1.85 | 1.00-3.39 | 0.047 |
| Stage I-II | | | | | | |
| KLK15 unadjusted | 7.10 | 1.47-34.21 | 0.014 | 1.35 | 0.65-2.81 | 0.33 |
| KLK15 adjusted^{d} | 3.18 | 0.58-17.39 | 0.18 | 1.59 | 0.43-5.81 | 0.48 |
| Stage III | | | | | | |
| KLK15 unadjusted | 1.93 | 1.24-3.02 | 0.004 | 1.74 | 1.014-2.99 | 0.044 |
| KLK15 adjusted^{d} | 2.11 | 1.31-3.38 | 0.002 | 1.79 | 1.00-3.21 | 0.048 |
| Optimal debulking | | | | | | |
| KLK15 unadjusted | 3.71 | 1.57-8.72 | 0.003 | 6.59 | 1.39-31.11 | 0.017 |
| KLK15 adjusted^{e} | 3.52 | 1.48-8.35 | 0.005 | 7.11 | 1.42-35.66 | 0.016 |
| Suboptimal debulking | | | | | | |
| KLK15 unadjusted | 1.52 | 0.93-2.48 | 0.09 | 1.21 | 0.67-2.14 | 0.52 |
| KLK15 adjusted^{e} | 2.03 | 1.17-3.51 | 0.01 | 1.53 | 0.82-2.87 | 0.17 |

^{a}Hazard ratio (HR) estimated from Cox proportional hazard regression model
^{b}Confidence interval of the estimated HR.
^{c}Multivariate models were adjusted for stage of disease, residual tumor, histologic type and age.
^{d}Multivariate models were adjusted for tumor grade, residual tumor, histologic type and age.
^{e}Multivariate models were adjusted for stage of disease, tumor grade, histologic type and age.

TABLE 9
Concentration of hK14 in various biological fluids

| | | Number of | hK14 concentration (μg/L) | | | | Positivity |
|---|---|---|---|---|---|---|---|
| Biological Fluid | | samples tested | Range | Mean (SD)¹ | | Median | (%) |
| Seminal plasma | | 36 | 0.6-23.6 | 10.8 | (6.0) | 10.7 | 100 |
| Amniotic fluid | | 46 | 0.5-19.8 | 4.6 | (4.7) | 2.6 | 100 |
| Follicular fluid | | 4 | 0-0.8 | 0.2 | (0.4) | 0 | 25 |
| Ascites fluid² | | 51 | 0-0.77 | 0.063 | (0.16) | 0 | 18 |
| Serum: | male | 27 | 0-0.16 | 0.02 | (0.05) | 0 | 11 |
| | female | 28 | 0 | 0 | (0) | 0 | 0 |

| | | | | | |
|---|---|---|---|---|---|
| Cerebrospinal fluid | 6 | 0 | 0 | 0 | 0 |
| Breast milk | 5 | 0 | 0 | 0 | 0 |

¹Standard deviation.
²From ovarian cancer patients

TABLE 10
Concentration of hK14 in sera of patients
with various malignancies.

| | Number of serum | Patients (%) with hK14 |
|---|---|---|
| Malignancy | samples | serum values > normal¹ |
| Ovarian cancer | 20 | 13 (65%) |
| Breast cancer | 20 | 8 (40%) |
| Prostate cancer | 31 | 5 (16%) |
| Colon cancer | 10 | 2 (20%) |
| Testicular cancer | 10 | 1 (10%) |

¹normal female = 0 μg/L; normal male ≦ 0.16 μg/L

TABLE 11
Descriptive statistics of the variables studied in breast tumors.

| | hK14 | hK3 | hK6 | HK10 | ER | PR | Age |
|---|---|---|---|---|---|---|---|
| | (ng/mg) | (pg/mg) | (ng/mg) | (ng/mg) | (fmole/mg) | (fmole/mg) | (years) |
| N | 341 | 341 | 341 | 333 | 341 | 341 | 341 |
| Mean | 0.18 | 172.27 | 4.84 | 0.22 | 94.51 | 120.04 | 63.05 |
| Std. Deviation | 1.22 | 822.78 | 17.25 | 0.72 | 127.89 | 175.43 | 14.84 |
| Median | 0.00 | 3.74 | 1.08 | 0.022 | 42.00 | 32.00 | 63.0 |
| Minimum | 0.00 | 0.00 | 0.00 | 0.00 | 0.00 | 0.00 | 27.0 |
| Maximum | 16.77 | 8620.7 | 184.05 | 7.52 | 680 | 920 | 99.0 |

TABLE 12
Association between hK14
status^{a} and steroid hormone receptor status^{b}

No. of patients (%)

| | | hK14- | hK14- | |
|---|---|---|---|---|
| Variable | Patients | negative | positive | p value |
| ER Status | | | | |
| Negative | 100 | 74 (74.0) | 26 (26.0) | 0.036^{c} |
| Positive | 241 | 202 (83.8) | 39 (16.2) | |
| PR Status | | | | |
| Negative | 128 | 101 (78.9) | 27 (21.1) | 0.46^{c} |
| Positive | 213 | 175 (82.2) | 38 (17.8) | |

1. (canceled)

2. A method of assessing whether a subject is afflicted with or has a pre-disposition for endocrine cancer, the method comprising comparing:
(a) levels of kallikrein 12, kallikrein 14 and/or kallikrein 15 or nucleic acids encoding kallikrein 12, kallikrein 14 and/or kallikrein 15 in a sample from the patient; and
(b) levels of kallikrein 12, kallikrein 14 and/or kallikrein 15 or nucleic acids encoding kallikrein 12, kallikrein 14 and/or kallikrein 15 in samples of the same type obtained from control subjects, wherein significantly altered levels of kallikrein 12, kallikrein 14 and/or kallikrein 15 or nucleic acids encoding kallikrein 12, kallikrein 14 and/or kallikrein 15 relative to the corresponding levels from control subjects of kallikrein 12, kallikrein 14 and/or kallikrein or nucleic acids encoding kallikrein 12, kallikrein 14 and/or kallikrein 15 is an indication that the patient is afflicted with endocrine cancer.

3. A method of claim 2 wherein levels of kallikrein 14 are compared to levels from control normal subjects and higher levels of kallikrein 14 relative to the levels for normal subjects is an indication that the patient is afflicted with breast or ovarian cancer.

4. A method for monitoring the progression of endocrine cancer in a subject the method comprising:
(a) detecting kallikrein 12, kallikrein 14, and/or kallikrein 15 proteins or nucleic acids encoding the proteins in a sample from the patient at a first time point;
(b) repeating step (a) at a subsequent point in time; and
(c) comparing the levels detected in (a) and (b), and therefrom monitoring the progression of the endocrine cancer.

5. (canceled)

6. (canceled)

7. A method of claim 2 for diagnosing and monitoring ovarian carcinoma in a subject comprising comparing kallikrein 12, kallikrein 14, and/or kallikrein 15 in a sample from the subject.

8. A method of claim 2 wherein the kallikrein 12, kallikrein 14, and/or kallikrein 15 is measured using antibodies specifically reactive with kallikrein 12, kallikrein 14, and/or kallikrein 15 or a part thereof.

9. A method of claim 2 comprising (a) obtaining a biological sample from a subject; (b) detecting the amount of kallikrein 12, kallikrein 14, and/or kallikrein 15 in said sample; and (c) comparing said amount of kallikrein 12, kallikrein 14, and/or kallikrein 15 detected to a predetermined standard, where detection of a level of kallikrein 12, kallikrein 14, and/or kallikrein 15 significantly different than that of a standard is indicative of endocrine cancer.

10. A method of claim 9 wherein levels of kallikrein 14 are compared with normal levels and higher levels of kallikrein 14 in the sample compared with the normal levels is indicative of ovarian cancer or breast cancer.

11. A method of claim 10 comprising
(a) incubating a biological sample from the subject with a first antibody specific for kallikrein 12, kallikrein 14, and/or kallikrein 15 which is directly or indirectly labeled with a detectable substance, and a second antibody specific for kallikrein 12, kallikrein 14, and/or kallikrein 15 which is immobilized;
(b) detecting the detectable substance thereby quantitating kallikrein 12, kallikrein 14, and/or kallikrein 15 in the biological sample; and
(c) comparing the quantitated kallikrein 12, kallikrein 14, and/or kallikrein 15 with levels for a predetermined standard.

12. A method of claim 2 for diagnosing and monitoring endocrine cancer in a sample from a subject comprising isolating nucleic acids, from the sample, and detecting KLK12, KLK14, and/or KLK15 nucleic acids in the sample.

13. A method as claimed in claim 12 whererin the presence of different levels of KLK12, KLK14, and/or KLK15 nucleic acids in the sample compared to a standard or control is indicative of disease, disease stage, and/or prognosis.

14. A method of claim 2 for determining the presence or absence of ovarian cancer in a subject comprising (a) contacting a sample obtained from the subject with an oligonucleotide that hybridizes to KLK12, KLK14, and/or KLK15;
and (b) detecting in the sample a level of polynucleotide that hybridizes to the KLK12, KLK14, and/or KLK15 relative to a predetermined cut-off value, and therefrom determining the presence or absence of ovarian cancer in the subject.

15. A method as claimed in claim 14, wherein the polynucleotide is mRNA and the level of polynucleotide is detected by polymerase chain reaction.

16. A method as claimed in claim 14 wherein the polynucleotide is mRNA and the amount of mRNA is detected using a hybridization technique, employing oligonucleotide probes that hybridize to KLK12, KLK14, and/or KLK15, or a complement of KLK12, KLK14, and/or KLK15.

17. A method of claim 16 comprising (a) contacting a sample obtained from the subject with oligonucleotides that hybridize to one or both of KLK14 or KLK15; and
(b) detecting in the sample levels of polynucleotides that hybridize to one or both of KLK14 or KLK15 relative to a predetermined cut-off value, and therefrom determining the presence or absence of ovarian cancer in the subject.

18. A method of claim 12 wherein one or both of KLK14 and KLK15 mRNA is detected by (a) isolating mRNA from a sample and combining the mRNA with reagents to convert it to cDNA; (b) treating the converted cDNA with amplification reaction reagents and nucleic acid primers that hybridize to one or both of KLK14 and KLK15, to produce amplification products; (d) analyzing the amplification products to detect amounts of one or both of KLK14 and KLK15 mRNA; and (e) comparing the amounts of mRNA to amounts detected against a panel of expected values for normal and malignant tissue derived using similar nucleic acid primers.

19. A method of claim 2 wherein higher levels of KLK14 in patients compared to a control is indicative of early stage disease (Grade I or II), optimal debulking, longer progression free disease and overall survival, and/or that the subject is responsive to chemotherapy.

20. A method of claim 2 wherein higher levels of KLK15 in patients compared to a control are indicative of ovarian cancer.

21. A method of claim 2 wherein higher levels of KLK15 in patients compared to a control are indicative of reduced progression free survival and overall survival.

22. (canceled)

23. (canceled)

24. (canceled)

25. (canceled)

26. (canceled)

27. (canceled)

28. A method of claim 2 wherein the sample comprises serum.

29. (canceled)

30. (canceled)

31. (canceled)

32. A kit for carrying out a method as claimed in claim 2.

33. (canceled)

34. (canceled)

35. (canceled)

36. (canceled)


SEQUENCE LISTING (31 sequences)

SEQ ID NO: 1 - protein, 254 aa, Homo sapiens
  ref <308>: DATABASE ACCESSION NUMBER: Genbank/AAF06065
  ref <309>: DATABASE ENTRY DATE: 1999-11-01
  ref <313>: RELEVANT RESIDUES: (1)..(254)
MGLSIFLLLCVLGLSQAATPKIFNGTECGRNSQPWQVGLFEGTSLRCGGVLIDHRWVLTAAHCSGSRYWVRLGEHSLSQLDWTEQIRHSGFSVTHPGYLGASTSHEHDLRLLRLRLPVRVTSSVQPLPLPNDCATAGTECHVSGWGITNHPRNPFPDLLQCLNLSIVSHATCHGVYPGRITSNMVCAGGVPGQDACQGDSGGPLVCGGVLQGLVSWGSVGPCGQDGIPGVYTYICNSTLVGLGTSWNFNSCQPF

SEQ ID NO: 2 - protein, 248 aa, homo sapiens
  ref <308>: DATABASE ACCESSION NUMBER: Genbank AAD26426
  ref <309>: DATABASE ENTRY DATE: 1999-11-01
  ref <313>: RELEVANT RESIDUES: (1)..(248)
MGLSIFLLLCVLGLSQAATPKIFNGTECGRNSQPWQVGLFEGTSLRCGGVLIDHRWVLTAAHCSGSRYWVRLGEHSLSQLDWTEQIRHSGFSVTHPGYLGASTSHEHDLRLLRLRLPVRVTSSVQPLPLPNDCATAGTECHVSGWGITNHPRNPFPDLLQCLNLSIVSHATCHGVYPGRITSNMVCAGGVPGQDACQGDSGGPLVCGGVLQGLVSWGSVGPCGQDGIPGVYTYICKYVDWIRMIMRNN

SEQ ID NO: 3 - protein, 111 aa, homo sapiens
  ref <308>: DATABASE ACCESSION NUMBER: Genbank/AAF06066
  ref <309>: DATABASE ENTRY DATE: 1999-11-01
  ref <313>: RELEVANT RESIDUES: (1)..(111)
MGLSIFLLLCVLGLSQAATPKIFNGTECGRNSQPWQVGLFEGTSLRCGGVLIDHRWVLTAAHCSGRPIPGSAPVPQPLHRLPCHLPWCVSRENHEQHGVCRRRPGAGCLPG

SEQ ID NO: 4 - DNA, 11820 bp, Homo sapiens
  ref <308>: DATABASE ACCESSION NUMBER: Genbank/ AF135025
  ref <309>: DATABASE ENTRY DATE: 1999-11-01
  ref <313>: RELEVANT RESIDUES: (1)..(11820)
attgaggggggatcccagcaggtcccatttgttcagattcttctgggcctttctgtgttgcatttcttcctctgcaggacacctgtcacatgagggtcttcaggggaaaaggaattctagtgtctgtgactgcttcaaaggagaagtaaggggagaggagaggaaagcaggaggaggttggagagaacctcttgttcctgaggtcttccaatctccttcagctcaaagcactcagcatgctgaagttccagactctgggctatcacgttctgacatgcaacacaggcaacaccccagctccatccacgtttctccaaaagcacaggcatgacgtcatatggtgacaaacacccttgtccaaaggaagcccataggatacgctaattctagattcacaaatactctagaggaactcacacaatgggatgggccagtgccccacacagagtatgaggcctcccaccttggttgaatatctttttcttttttttctttttttttttttttgagacggagtctcactctgtcacccaggctggagtgcagtggctcgatctcggctcactgcaacctctgcctcccaggttcaagtgattctcctgcctcagcctctggagtagctggaattacaggtgcccaccaccacaactggctaatttttgtatttttagtagagacgagggtccaccatgttgaccaggcttgtctcgaactcctggtctcaagtgatctgcccacctcggcctagtgctgggattacagacgtgagtcaccacgcccggccccaccttggttaaatttctgaaaatcatttggtaaagtgaggacccctccagctgagacactgccaggaaacagctattgagtctcttagcacccacagcattaaaacaaacccaaaacattttaggcctcgttgagttctggaggcaaaatattcctcatctacaaatttattttattttattttttaataaaagttattattatttttttttatagagagacagggtcttgctctgtgacccagactagagtgcagtggtgtgaccatggttcactgcagcctccacctcctgggctcaagcgatcctcccacctcagcctgccaagtagctgggaccacaggtatgcccctcccccaggctaacttttttttactttttgtggagatgaggtctcactatgttgcccatgctggtcttttttttttttttttttttttttgagacgggagtgcaggctggagtgcccaggctggagtgcagtggcacaatcataactcactgcagcctcaaactcctggggtcaagtgatcctcccacctcagcctcccacataactaggactacaggcctgtgtcaccataatgcctggttaatttttttagtttttgtttgtagaaacagggtctcactatgttacccaggctggcctctaactcctggcctcaatccatcctcccatctcaacctcccaaagtgctaggattatagccacgagccaccatgcccggccccatacttatatttacttgtagtgagaacacttaaaccctactcggtcagtaattttcaagtacacaatacattgttactaactatatacatggtatttttaatttgcatgaatgctccttaaaatcagcgagcccgccttattttttgtattcaattttattggatataaattccacatcagtaaacctgattctcttaaaaatctacacagaaaaaaaaaaagagagagaggttgagttgggtagttgttggtatttttgtttttgtttctttgcttattcagttgaggtttattgtaaaacttgacaccagaaaaaggaagaagtggcgtttttgtgctgtgagcgtgtgacgtgtgtcctttccagagaaaggacagtcatggtgcttttttatcctctctgcccaaagaaggaaagctcttaacagccagcaggaggctttgtagggaccagcgttatcaacgccagtcgcgctgaccaatgatgagttaaagcagttaggtcgtttctaagagcaaatcaaaagctaaggttctgtgattctgaaaatgagacacggacagagactggagacccagagagaaagtgaaggactaaaagacagtcatagggtgggagtttgctctcctctgttttgttctgggtttttttgtttgtttgtgcgctctgtctgaccgcttttcttttttttctttttctttcttttttttttttttttttttgagatggagtcttgctctgccgcccaggctggagtgcggtggcacaatctcggctcactgcaagctccgcctcccacgttcaagggattctcctgcctcagcctcccgagcagctgggattacaggcatgcaccaccacactcagctaatttttgtatttttagtagagatggggtttcaccatattgaccaggctggtcttgaactcctggcgtcaagtgacttgcccgcctcggcctcccaaagtgttgggattacaggcgtgagccaccgcgcctggccctgactgcttttctccttggtttgtttgtcaatcccccttcctctgagccgaattccctttttgttctcattttctctctctgtcccctctctctctccttctttctctccttccattcctcctagatgaagcaaaaactcagataaaccagcacagaggccaggtatggtggctcacacctgtaatcccggcactttgggaagccaaggcaggcaggttgcttgaggccaggagttcaagaccagcctggcccacatggtgaaaccccgtctctactaaaaatacaaaaattagccggacatggtggcacgtgcctgtaatcccaggtactcaagaggtggaggttgcagtgagcggagatcacagccctgcactccagcctgggtgacagagcgagactccatctcaaaacgaaaaacaaaaaacagcacaaagttcccttgtcctgtgactcattctctctctctctttctaccatttctccttccctgtgtcttttttttttctctctgtgggttttatttaagcaatagaagttcttagcaaagaaaaactttatggaattagattgatccacttcatatgtacatatatgaactcagttcagaaactctcttctacccctgcctgatcacctatttggaagtctgttccttcaactcttcttctctttctgggactctttctagcttgggcttcctgcccctcccgtccactctcctgctttcacagcctctccttccccctgcccctcccctgcactgcatggggatgggccccaggtgtccaaggtctccccaccctcctttgtcactggagtcaggattagaacccagctccctagtcaccttgagtcatcagtcctggggctgctgacgggcttgcagaggagagagggagtggggctgggtcttcccaccctgggtcctttcctccttccccactccgtttagctgtaaagctcaattaagtgtgattagctgagaagagtttctgcagaattagagcacgccccacccctgtcttcgtggtccccttcccttaacccggaaactggatgggccaggacaaagagagttaagagctttgtcagtggtctgtctggagcgacagatggaaggaaagggaccggttgagcaacatgacaggtggctgaggagccaggtgcagagtggtagagttggctggcggagtggccagcacatgagaagacaggcaggtaggtggacggagagatagcagcgacgaggacaggccaaacagtgacagccacgtagaggatctggcagacaaagagacaaggtgagaaggaggtaggcgactgccaatgagggagtgacacacaggggagcaggtagagagaggacaagcaggtcatccccttggtgaccttcaaagagaagcagagagggcagaggtggggggcacagggaaagggtgacctctgagattccccttttcccccagactttggaagtgacccaccatggggctcagcatctttttgctcctgtgtgttcttggtgagttctcccggagcagggagagggcaggactgcgactggatcccttcacccccatgaggaggccccaccaccctccccatctcagctctggcccccagcctggtggtgaggaggagaggggctttctctgtgcctccatttacctgcagctctcagggtactgctcacctcggtctcccctattttttgatccctcttcccttctgtccctctctgaatctctgtctctccatttccctcctatgtgtaagcatctttctccctgggtgtctttgatgtttcatggtctttttctatcactgggtctctctctctttctctctctttctcgtctctctttctcctctctctctcctgcctgtttctctctctcactctgtgtgtctctccatctctgtatcttttcttcctctctctgacccatgcccctgtctgtctccagggctcagccaggcagccacaccgaagattttcaatggcactgagtgtgggcgtaactcacagccgtggcaggtggggctgtttgagggcaccagcctgcgctgcgggggtgtccttattgaccacaggtgggtcctcacagcggctcactgcagcggcaggtaagtcccttcctggggtgggcgaagggaggactatgggaaggcaagcgctgggggtaggatcacaagggagggtggtgcccactgggaagaagctgatcctgcaacaagagagtctgaggttagaccaggagtggaacttccttagcagtgggcctggggtggtgctgggcagggtgaggtatgttgggtggagggccggggagggtcctggaacctgccctcctgcctctcccattcctgcatgtaccctttctttcctatatgacatctgccactcaccccagccattccttgacccagtctgggcccggggcccaggtctcacccaagctctttttctttttcttttttttatttttttgagacagggtctcgctctgtcgcccaggctgctgtgcaatggcgtgatcacagctcactgctgtctctgcctcccaggttcaagtgattctcctgccccagcctcctgagtagctgggattacaggcacccgccaccatgcccagctaatttttgtattttttgtagagacagggttttgccatgttggccaggctggtctcgaactcctggcctcaaatgacctgcccgtcttggcctcccaaagtgctgggattacaggtgtgagccactgcacccggccaacatgacccaaactctttgtgcaacttcagaatctatgcctggcacctctctgggcctcagtagactgatgttctggaatttttttctttttctttctttttttttttttttggagacagagtcttgctctttctgtcatccaagctggagtgcagtgatgctatcttggctcactacagcctcaaccacctgggctcaagtgatcctcacacctcagcctcccaaggagctaagactacaggcctgcgccaccacacctggctaatttttaaattttttttgtagagacagggttttgctatgttacccaggctggtctcaaactcctcagctcaagcaatcttcctgccttgacctcccaaagtgctgggattacaggcatgagccactgtgcctggcctggaactttttttgtgaaaggggagatcagatgcaaagaaacagagactcagggagagagagggccagcagcaggatgcagagaggccattcatcaacccactcgttcaatcatgaacccactcgtccacgcatgagcatggagggcacatgctccgtgccaggcggtgggaataaggcagtgaacaaggtccactgatgtccctgccttcatgggcttcaccagccgagagaatcagaaagagaggcctggcgcggtggctcacacctgtaatcccagcactttgggaggccgaggcgggcggatcacttgaggtcaggagtttgagaccagcctgacacacatggtgaaaccttatctctactaaaaatacaaaaattagctgggcatggtggcatgcttctgtaatcccagctacttgggaggctgaggcaggtgaattgcttgaacctgggaggtggaggttgtagtgagccaagatggtgccactgcactccagcctgggcgacagagcgagactcggtcttgaaaaaaaaaaaaaaaaaaaaaaggagagagagagacacagatgcagggacatggtaggagaaacagggaacacccaagatggaaagagggtgatggaggttgggaataagagcctgtaagagagactcggagaatgagagttgcgggtgagaggacagacagtgaggggcagaacagtggggagcggcaggagcgcctgagtgtccgtggaggggtgcaaggtgggggactgcgtgcctgccacccgctcagccgtcgccaccggcagcaggtactgggtgcgcctgggggaacacagcctcagccagctcgactggaccgagcagatccggcacagcggcttctctgtgacccatcccggctacctgggagcctcgacgagccacgagcacgacctccggctgctgcggctgcgcctgcccgtccgcgtaaccagcagcgttcaacccctgcccctgcccaatgactgtgcaaccgctggcaccgagtgccacgtctcaggctggggcatcaccaaccacccacggagtaaggggcccagggccaggggtcaggggtcaggatgggtacaagtctgggatgcagggcgagaggtcgaatcatgacacctcagaggaaggatgggtaaagggtcagggtgtgggatgggacatcaggatcatggtttggggtcagagattatggtggattggggtcttgggagccaaaggggttaaaggactgggtatgaagtcagggatcagaggtcagaggtcagagtgtgtcagaggtcatcacactggagcaaaaggcatatatatatatatatgtatgtataggatatgggcattgtgggtcatgggtctggggttagaggtcaccgtagaattaaggtcatgggatccagaggttgtacaatctggtcaaaatctgaggatggaaattgggattctatccaaaatcacatatctgagattggaggtcatagcgtttggggtgtggggcccgaagtttggggtcatggaggctggggcccaataaactaggatcaggggacactggcgttggaagcagtgaggtttggaagatgcagagctgaggttggaggttaaggtaaagacagggacatggggtcaggagacagaagatatgagatcaagctgggatcataaggtaataagacagaaggtcaaagatcacagtagctggcattgaagagggtcaggtctggattcgttgtctctgacgctggagagacaagaaagttcttgagttatgccactcaaagtcaaatgtcaaagatcaaagagaccgtcaatcatctggggtcatgattcatatgaaattaagtcataaatatgtaacttggaggtttcgggattgtagtacaggtcggtgaggggcaggggtattgacatggatgggccacatccagggaagagggacgtggcctcaaagtggggagatttaggggaccctgcagcacgcatgttctctctccagacccattcccggatctgctccagtgcctcaacctctccatcgtctcccatgccacctgccatggtgtgtatcccgggagaatcacgagcaacatggtgtgtgcaggcggcgtcccggggcaggatgcctgccaggtgagccagtgcaggcagcgtgcgtggtcaccaggacaggaagtgaaggggaggggctggaagcaggaggggaactgatggaggatgaatcagggaaaggggatgctgcagagagacggggtcaaaaaggaagggagaggctggttacggaggctcacacctgtaatcccagcactttgggaggccgaggcgggcggatcacttgaggtcaggagttcaagacaagcctggccaacacggtgagactctgaatctactaaaaataccagaattagccgggggtggtggtgcaagcctgtggccccagctacttggaaggctgaggcaggagaatcgcttgatcccgggaggcggaggttgcagtgagctgagatcacgccactgcactccagcctgggcgacagagccagactctgtctcaaaacaaaataattaataataataataataataataataataataatggaggagaggcccaggataagggagggagagagacagggagtaaaagggaggaccggggaatggaggagggggaggggcagggagagagagggaggaagggaacagagaaggaaagatggggcaggggttacagagagagacagcaaaacagacggagaggactgggagcccagacagggaaccagctgtttctggggctctaagtctttcccataccatcctccagttggtgctgtcccagactgagagagatttgaggatggcggtctctcccctcattggtcagggccccagccattgtccttgagagaactctgtgcttttgatggagtcctgcccaccttccctgggattggtcatttttgatggcactctctcccctcattggtcagaaccccaggcattgtccttgagagaacctctatcctttatggagtcccaccctcctcccctgggattggtcattgataatagtgttctctctcctcattggtcagggccccagccattgtccttgagagaatgctcgactctttatgttgtcttgacagcctcccctgagattggtcattaatgactgtgctctctctcctcattggtcagggccccagccattgtccttgagagaacctctgtcctttatggagttccacccttcttccctgggattggcccctagagacagtggttcttctcttttggttagccattgccattgtcctccgggaaagtgattatactcttttgtctaatgaccagacttggagccctccccaaggcccaggactgggttgaagggttggggaggaaaacagaaataagatgtctcccttgttcagacagtacttctcttcccttccagggtgattctgggggccccctggtgtgtgggggagtccttcaaggtctggtgtcctgggggtctgtggggccctgtggacaagatggcatccctggagtctacacctatatttgcaagtatgtggactggatccggatgatcatgaggaacaactgacctgtttcctccacctccacccccaccccttaacttgggtacccctctggccctcagagcaccaatatctcctccatcacttcccctagctccactcttgttggcctgggaacttcttggaactttaactcctgccagcccttctaagacccacgagcggggtgagagaagtgtgcaatagtctggaataaatataaatgaaggaggggccatgtctgtccatttgaagtcctcatgctggttgagactggaagaaggactcagcagtttccctatctcataggagtagaaacagagctcaaataaggccaggcacagtggctcacacctgtaatcccatcactttgggaagctgaggcaggtggatcacctgaggtcaggaactcgggaccagcctggtcaacatagtgaaaccccaactctactaaaaatgcaaaaattagccaggcatggtggcgcatgcctgtaatcccagctactcaggaggctgagacaggagaatagcatgaacccgtgaggcagaggctgcagcgagccgagattgaaccattacactccagcctgggcgacagagcgagactccatctcaaaaacaaacaaacaaaaaacccagtgctcaaataggatgagggtcttccctgagtagttactcagaaatggagtagaaaaagttacttttaataatataggccgggtgcagtggcccacgcctgtaatcccagcactttgggaggccgaggtgggaggatggcttgagctcagatttcgagatcagcctggcaacacagtgaaatcttgtcactacaaaaacacaaaaaattagctgggtgtggtggtgcgtgcctgtagtcccagctacttgggaagctgaggtgggaggatcacccgagccggggaggtggaggctgcaaagagccgagatcatgccactgcactccagcctgggcaataaagtgagaccttgtctcaaaaacaaaaacccagcaatataaataagacacatgtttcttcatctggcataatagaaatagtgcccagagcttataagcttttcaagagtccacaaaagacccgaaaaagaaaaagaaaattgttagctccaaaataccagatgaaagctgcaaagtcaacatttatgaccatttaatccaatgtccataaaacgtagcattctttccactagccaactgcagtttactttcttgtaatgaagcatacattgtatctttaatgtgggacgtggctttgttctaataagacgaagggtggagtgcaggcttggaaagcaggagagctcagcctacgtctttaatcctcctgcccaccccttggattctgtctccactgggactcaagaggtgaggagagaccatctccccaaatgcactgaagggaaactggaggagggagggagtgaggggtgatcataccagcggaggcacatttgctgagcccccccgcagtctgctctttccaagtggaccctcctggaagcctgatcccaacctcccctgcaagcaggtctgtcacccccatctctcagatgaagaaactgagccttgcaggggtggagtcccttgtccccacgtcataagggtagtcatagtagtaggaagaggaagcacctaggtttgaggccagggctggctgctgtcagaacctaggccctcccctgccttgctccacacctggtcaggggagagaggggaggaaagccaagggaagggacctaactgaaaacaaacaagctgggagaagcaggaatctgcgctcgggttccgcagatgcagaggttgaggtggctgcgggactggaagtcatcgggcagaggtctcacagcagccagtaagtgaacagctggactcgggctgcctgggcggcagggagaagcgggcaggggaagggtcagcagaggagcgaggccccagaggagccctggggtggagcacagccaagggctctgttccctttcctggactcggcttccacaggccctgacctgcctcccccaccctccggtcctgcccctgtgcctggcagcagccccacctgtgtgacatcccagcacaccccccctctccttgcaaaggagaagggagcggcctaggggaggccaggggcccacctgggctggggctgtggagagggagtggctgggacgggaggaaaaagagagacggagattagatggaagaagagggatttcaagacaaattgccagagatgcagtcagagagactgactgagagacacaaagatagaaggaattagagaaagggccacacagagccagacagagagagaagagtggagatggagacagggacgaggacagagaaaggcagacagacacatagggacagaaagagaaaaatcacacaaagtcagaattactgaatgacagggaatgacacatagaacgagacacagattcagagactcagggcagggaaaggaaggctgcagacagacagacagacagaggga

SEQ ID NO: 5 - protein, 251 aa, Homo sapiens
  ref <308>: DATABASE ACCESSION NUMBER: Genbank/ AAK48524
  ref <309>: DATABASE ENTRY DATE: 2000-06-30
  ref <313>: RELEVANT RESIDUES: (1)..(251)
  ref <308>: DATABASE ACCESSION NUMBER: Genbank/ AAD50773
  ref <309>: DATABASE ENTRY DATE: 2000-01-19
  ref <313>: RELEVANT RESIDUES: (1)..(251)
MFLLLTALQVLAIAMTQSQEDENKIIGGHTCTRSSQPWQAALLAGPRRRFLCGGALLSGQWVITAAHCGRPILQVALGKHNLRRWEATQQVLRVVRQVTHPNYNSRTHDNDLMLLQLQQPARIGRAVRPIEVTQACASPGTSCRVSGWGTISSPIARYPASLQCVNINISPDEVCQKAYPRTITPGMVCAGVPQGGKDSCQGDSGGPLVCRGQLQGLVSWGMERCALPGYPGVYTNLCKYRSWIEETMRDK

SEQ ID NO: 6 - DNA, 8280 bp, Homo sapiens
  ref <308>: DATABASE ACCESSION NUMBER: Genbank/ AF161221
  ref <309>: DATABASE ENTRY DATE: 1999-06-21
  ref <313>: RELEVANT RESIDUES: (1)..(8280)
atcgtgtaatcaccgccacatccagtgcaaagctgattcgtcaccacagagcagctccctcctgccaccccatccctgggtcccaagagaaccctttcttaaaagagggagttcttgacgggtgtggtggctcatgcctgtaatccttgcactttgggaggccaaggagggtggatcatttgaggtcaggagtttgagaccagactggccaacatggtgaaaccctgtctttactaaaaatacaaaaaaatgagcggggcatggtggtgggtgcctatagccccagctactcaggaggctgaggcaggagaatcgcttgaacccaggaggcagaggttgcagtgagccgagattgagccactgcactccagccggggctaaagagtgagactctgtctcaaaaaaaaaaaaaagaaaaagaaaaaaagaaaaaaaaataaaataaataaataaataaaataaatttaaaaatttaaaaataaagagggggttcttgtgttgatgccgagcctgaaccaaggcagaggaggccgggaaggcttcccaaggccttcagctcaaagcagggaggcccatagttaaacagaaacagttcaggaatcacagaaaggcacctggggagagatgggtgtgtggctccagatgcaggtgcccagacagtgcgtccccaggtgtacagacagacccaggccaagctccagctcaaagagccagcctaggggggtgccgaggtggagggaggctgagtcaggctgaggccggggaacagttggggtagccaagggaggcaagcagcctcctgagtcaccacgtggtccaggtacggggctgcccaggcccagagacggacacaagcactggggaatttaaggggctaggggaggggctgaggagggtaggccctcccccaaatgaggatggaacccccccaactccagaacccccctgcaggctggccagaatccttccccatctcattcactctgtctctcctgctctctgccgtctcctattttgaatttccaaccccgtctgttaagactgtccttctgtctctgaatctctgtccccttctctttctgggtctctctccctctccctctgggtctctgtccccctctctgggtctctgtcactctctctttgcatctccagctctcactttgtctctgcacctagcagatcccaagctggggaatgccagttctggcaccaaccttcctgctccctgctggggcctctgctcccccatctctcaggagtcgaaagtgagaaagcaaggtgggcagctctgctccaggtccaggtatctcccgcccacctcctgcccgtcctctatcccacccctcctctccatctctccctggcgctgccatctctcatctaggcctccgtctcctctgtcattgtccccatcccctgtaggtgcccatccttcccgtctcccctctgccatcggcctgcctgtcccatcctctttctcccaccatgtcccgttctcttccacgtctcatgcccgcactgccttcatcatcatcgctgttgttctgtgtgtgtttgtggtgagtgccgcatggtgggggcgtctcggcctctctcctctctctccactgttttctctttctgtgtgtctgtttccattctatctccaccttcttccctccgtcttttgcttttctatctccacttctccacacccctctctccctgcgtctctgtgtctccctcttcctctgtcttgtttttttcccaccgtctgcctcttctgttccctgtcacatccaacttccaccggtttctccagctctctcctcagttccttctctcatgagcacacctgcctctgtgctcgtattcctggactcctctctctccactgtcatatcttctcattcattttcccagtctctctctgtctcttgctctccccctctctgtcactctgtctctgtctctctctttctctctctctctctgtgtctctctgtctggctctctctctgtctctctctccatctctctctctctctcccccccgtcaccctgtctctgtctctctctgtctgtgtgtctctctgtctttctctctctccatctctctctgtctctctctctctctctctctctctccctctctccctcctcccgtgactccctctctcagtccatctcttcctccctctctcagccccttcgtgccctttcctctgacactccccaccctggtttcctgactccaccactagatccaccacctccagcaactgggaaccctcccctgcccaccctgccctggggtcccctcccaggattccttctagattatagcatcttccctgggcgggttctcatgaacaattgtggctgcttttttggccagacaggggagggaggggatgggatcagggagtcctggaatgggaactaggcaataaaaaaaaaaaaatgtcagaagcagggcggcgggaggtgggggcagggccagctgtccttaccagggataaaaggctttgccagtgtgactaggaagagagacacctcccctccttccttcatcaagacatcaaggagggacctgtgccctgctccacatcctcccacctgccgcccgcagagcctgcaggccccgcccccctcgtctctggtccctacctctctgctgtgtcttcatgtccctgagggtcttgggctctgggtaagtgccccttgctgtctctgcctctcagcccccggttctgttgaaggttccttctctctcactttttctctgcatttgacaggacctggccctcagcccctaaaatgttcctcctgctgacagcacttcaagtcctggctataggtaagagaacggttgggtatgacacaagggggtcccctggagactctgagaagagatggggatgggtccttggggcccctggatgctcatggtgacctcataagaaagagcagggagtggtttgggggtcatggtgggggaacgtgctggaggcctaaattcctagttgtggaggtgctagggaattgtggggccggggagagaggtgtttataaggtctggtgcaaaatacataaggaatcttagggaactattaggtcctgagtgggtcatagcagaaagatcacggggctctacctgactgtgttaggaaagaaacaatgtcagaaagatgttttgttgtcagagggaaggtggagaaggatgatgggatggcgggatcgtggcatggggtggcgggatcgtggcatgggtgtgtgaggtggatgggggcaagtgtggggcaagagatggcggatccttggggtcccactgagtgggaacgttggggaggagacagggaggtccttgaatgtgttggggaaggactcattggggggaaatgtggcatatttcgagaagtgatcacagaaattatgggagcatagagctaagggtcgtagatgtagcaaggccctggataaggtggccacggcacaaaataagagatgctacggaggtgacttgggaggtgagtcagaaagctctccgtgctggggcaataacggggtcaatattgggcatgtctcaccctgggtgggacagatagaggcgggcagtttaggggttagaccaaaaggaaggggatttgtcagttttggaatcctacaaacttgtggagtggagagtgtttgctcatctactttccccacccaatcctgtccactcctagccatgacacagagccaagaggatgagaacaagataattggtggccatacgtgcacccggagctcccagccgtggcaggcggccctgctggcgggtcccaggcgccgcttcctctgcggaggcgccctgctttcaggccagtgggtcatcactgctgctcactgcggccgcccgtaagtgaccccctcccctgtccctgtacctagtgaattccagagtctaaagccctagagctgagctgagaacctggatctctgtatagaacccaatgtagtggctggctcctggtttgaggtctagagaagagcctggaacaaaaacacagctcgggatgtgggctcctccataaatctcgaactcagcataggttctgaaagcagatgggcagcttggaacccatggacctgctgagaaccgaacatctgatccagtgattcttccagaggccacacattacatcgagaccaagcttagcccattccagattggtggctgaattcaggaccccgtctacattcagaaactcaggacactacgtagaactcagagcccagttcaggacctgcagtctagccataaatccagaactagaacgctgctcacagctggaacatacaactctaagaatagaggcaaaacctggaggctgtttcacacccaaggtttagttcagagtctagtctatagctccgctatgagcagacttcaacccagtgtttgaatcccagaatgtggcgggtgcggtggctcatgcctataatcctagcactttgggatgctgaggcaggcagatcacctgaggtcaggagttcgagaccagcctgagcaacatagagaaaccctgtctctactaaaaatgcaaaattagccaggcatggtggcacatgcctgtaatcccagccactcgggaggctgaggcaggagaatcacttgaacctgggaggcggaggttgcagtgagtcaagatcgcaccattgcactccaggctaggcaacaagagcgaaactccatatcaatcaatcaatcaataaatcccagaatgcagatcctaatcagaagccccatataaaacctagacccctcctaaattctagatctgaacttacaacccagaccccagccaagaggtcaaaatgcctataagccatatctatgccataaacaggtcagtctagaacctagagatcaaagctcaggccagagtctagaatataaaggccagaatgcaaaccagactctagaatcttggatccgggccataacctagagctccaactagaacccagagcccaacctgaggtcaagggctagggccagagtccagaaccaagagccctataatccaatatgaaacagacctgtagaggctgggtgcggtggctcacgcctgtaatcccagcactttgggaggctgaggcgggagaatcacttgaactgggagttggaggtcgagagtgagctgagatcgtgccactgcactccagcctaggtgacagagcgagactccatcacaaaaaaaaaataaataaataaatcaagtcataatccaggttcgatctagaatcctgatcttagcatagagtcaaaagtttaagatgtctagaactcagaacccaggctagaaacagaatggtgcctactccggaatatcagttccgatttagagcctagactcataacgcagtttcgcttaggactcaatgcaccgagcccagcacagaccctggcacggagccaagctctcccaatcatcaccttcttcccaagccaggagctggagcccagcccaagagcggaaggagaggcagctggggctgggccgagagaatgccctggccatggggaagggcacaggaggccaagaatgctcggcctgcagttagtgagaagcaggctagacctcggggaagactcgtcacccggccagggaaccgggctggagggtggggaggagtctctggctcagaccctgagcagcgcttctcttgggggtcgtggccaggatccttcaggttgccctgggcaagcacaacctgaggaggtgggaggccacccagcaggtgctgcgcgtggttcgtcaggtgacgcaccccaactacaactcccggacccacgacaacgacctcatgctgctgcagctacagcagcccgcacggatcgggagggcagtcaggcccattgaggtcacccaggcctgtgccagccccgggacctcctgccgagtgtcaggctggggaactatatccagccccatcggtgaggactcctgcgtcttggaaagcaggggactgggcctgggctcctgggtctccaggaggtggagctggggggactggggctcctgggtctgagggaggaggggctgggcctggactcctgggtctgagggaggagggggctgaggcctggactcctgggtctcaaggaggaggagctgggcctggactcatacgtctgagggaggaggggctggagcctggactcctgggtctcaaggaggaggggctgggcctggacttctgggtctgagggaggaggggctggggacctggactcccgggtctgagggaggagggactgggggtctggactcctgggtctgagggaggaggggctgggggcctggactcctgggtctgagggaggaggtgctggggctggactcctgggtcggaaggaggaggggctgggggcctggacccttgggtcttatgggagggtagacccagttataaccctgcagtgtcccccagccaggtaccccgcctctctgcaatgcgtgaacatcaacatctccccggatgaggtgtgccagaaggcctatcctagaaccatcacgcctggcatggtctgtgcaggagttccccagggcgggaaggactcttgtcaggtaaggcccaggatgggagctgtggtagggattatttgggactgggatttaagcaaatgatgtcaggagcatggaagtctgcagaggtcttcagaagagagtgaaccgcaggcacagagagattccgatagccaggccaccctgcttcctagccctgtgccccctgggtaatggactcagagcattcatgcctcagtttcctcatctgtcaggtgggagtaaccctcttagggtagttggtggaatgggatgaggcaggttggggaaagatcgcagagtggcctctgctcatatgggtctgggaaaggctgtgctgaggcttctagaaatcttaatgcatccttgagggaggcagagatggggaaatagaaaaagagagacacacaaatgttctacagttggagcgaacagagaggggcctggtgagattcaagggacaggcaggtgcacacagagacagagccagacccagcggagagggaaggaagtgccccgacctccggggctgagacctcagagctggggcaggactgtgtccctaactgtccaccagtgtctctgcctgtctccctgtgtctgcttctcgggttctctgtgccatggtggctctggctacctgtccatcagtgtctccatttctgttcctccccctcagggtgactctgggggacccctggtgtgcagaggacagctccagggcctcgtgtcttggggaatggagcgctgcgccctgcctggctaccccggtgtctacaccaacctgtgcaagtacagaagctggattgaggaaacgatgcgggacaaatgatggtcttcacggtgggatggacctcgtcagctgcccaggccctcctctctctactcaggacccaggagtccaggccccagcccctcctccctcagacccaggagtccaggcccccagcccctcctccctcagacccgggagtccaggcccccagcccctcctccctcagacccaggagtccaggccccagcccctcctccctcagacccgggagtccaggcccccagcccctcctccctcagacccaggagtccaggccccagtccctcctccctcagacccaggagtccaggcccccagcccctcctccctcagacccaggaatccaggcccagcccctcctccctcagacccaggagccccagtcccccagcccctcctccttgagacccaggagtccaggcccagcccctcctccctcagacccaggagccccagtccccagcatcctgatctttactccggctctgatctctcctttcccagagcagttgcttcaggcgttttctccccaccaagcccccacccttgctgtgtcaccatcactactcaagaccggaggcacagagggcaggagcacagaccccttaaaccggcattgtattccaaagacgacaatttttaacacgcttagtgtctctaaaaaccgaataaataatgacaataaaaatggaatcatcctaaattgtattcattcatccatgtgtttactttttattttttgagacaaggtcttgctcagtctcctggtgaaatgctgtaacgcaatcatagctcactgcaaccgtgacctcctgggctccagtgatcctcttacctcagcctcccgagtagctgggaccacaggtgcccgtcaccatgccccgctac

SEQ ID NO: 7 - protein, 256 aa, Homo sapiens
  ref <308>: DATABASE ACCESSION NUMBER: Genbank AAG09469
  ref <309>: DATABASE ENTRY DATE: 2000-03-06
  ref <313>: RELEVANT RESIDUES: (1)..(256)
MWLLLTLSFLLASTAAQDGDKLLEGDECAPHSQPWQVALYERGRFNCGASLISPHWVLSAAHCQSRFMRVRLGEHNLRKRDGPEQLRTTSRVIPHPRYEARSHRNDIMLLRLVQPARLNPQVRPAVLPTRCPHPGEACVVSGWGLVSHNEPGTAGSPRSQVSLPDTLHCANISIISDTSCDKSYPGRLTNTMVCAGAEGRGAESCEGDSGGPLVCGGILQGIVSWGDVPCDNTTKPGVYTKVCHYLEWIRETMKRN

SEQ ID NO: 8 - protein, 171 aa, Homo sapiens
  ref <308>: DATABASE ACCESSION NUMBER: Genbank/ AAG09472
  ref <309>: DATABASE ENTRY DATE: 2000-03-06
  ref <313>: RELEVANT RESIDUES: (1)..(171)
MWLLLTLSFLLASTAAQDGDKLLEGDECAPHSQPWQVALYERGRFNCGASLISPHWVLSAAHCQSRFMRVRLGEHNLRKRDGPEQLRTTSRVIPHPRYEARSHRNDIMLLRLVQPARLNPQGDSGGPLVCGGILQGIVSWGDVPCDNTTKPGVYTKVCHYLEWIRETMKRN

SEQ ID NO: 9 - protein, 161 aa, Homo sapiens
  ref <308>: DATABASE ACCESSION NUMBER: Genbank/ AAG09471
  ref <309>: DATABASE ENTRY DATE: 2000-03-06
  ref <313>: RELEVANT RESIDUES: (1)..(161)
MWLLLTLSFLLASTAAQDGDKLLEGDECAPHSQPWQVALYERGRFNCGASLISPHWVLSAAHCQSRFMRVRLGEHNLRKRDGPEQLRTTSRVIPHPRYEARSHRNDIMLLRLVQPARLNPQVRPAVLPTRCPHPGEACVVSGWGLVSHNEPGTAGSPRSQG

SEQ ID NO: 10 - protein, 121 aa, Homo sapiens
  ref <308>: DATABASE ACCESSION NUMBER: Genbank/ AAG09470
  ref <309>: DATABASE ENTRY DATE: 2000-03-06
  ref <313>: RELEVANT RESIDUES: (1)..(121)
MWLLLTLSFLLASTAAQDGDKLLEGDECAPHSQPWQVALYERGRFNCGASLISPHWVLSAAHCQSRFMRVRLGEHNLRKRDGPEQLRTTSRVIPHPRYEARSHRNDIMLLRLVQPARLNPQ

SEQ ID NO: 11 - DNA, 8735 bp, Homo sapiens
  ref <308>: DATABASE ACCESSION NUMBER: Genbank/ AF242195
  ref <309>: DATABASE ENTRY DATE: 2000-03-06
  ref <313>: RELEVANT RESIDUES: (1)..(8735)
agaatgggtgctgtgggattcaggggagacacctgttaggtgttggggcctcccagaagaggtgggggcagagtgtcagaggacaaagatgaatttggaagatatggggaagaaggatttcaattcaccctcaaagcttcctgaggcctcccgtgggtcgggccctgcagtactggagacccagagtggagtcagaccagctcctcggggagctgccagtctcgtaggggaggcagacaccactgagggtcaggggaggtcagagaaggcctcaaggaggaagcggggctggaagggaatggcgttggatatgcggtgggaggaatagcctaagcatgaaatggcaggagggaaaatggcagcactggctgcgtctaggacaaggtcatgggagacccagggagaggggctggaagggaagaagccacttttgtccttgaaagtgaggctggagccaggcaactcatgcctgtaatcccagcactttgggaggctgaggcgggtggatcactagaggtcaggagttcaagaccagcctggccaacatggtgaaactccgtctctactaaaattacaaaaattagctgggcgtggtggcacacacctgtaatcccaattgcttgggaggctgaggcaggagaatctcttgaacccagaaggcagaggttacagtgagcggagatcacgccactccactccaacctgggctacagagccagactccgtctcaaaaaaaaaaaaaaaaaagaaaaaaaaagaaagaaagtgaatttgaagagctggactttatcctggtggtgccaaggatccatggagggtggtgagcaggggaggggcacagccagctccagatgtagaaagaccctttggggtcatggctggagggcaagctggtggaggggactggactggagggggacccaaaaggccagataagagggttgagatagaccaggcgcggtggctcatgcctgtaatcccagcactttgggaggccgaggtgggtggatcatgaagtcaagagattgaggccatcctggctaacacggtgaaaccctgtctctacttaaaaaaaaaaaatttccaaaaaattagccgggcacggtggtgggcgcctgtagtcccagctactcgggaggctgaggcgggagaatggtgtgaacctgggaggtggagcttgcagtgagccgacattgtgccactgcactccagcctgggtgacagagtgagactccgtctcaaaaaaataaaaaaagttgggacagggggtccttgcgtgatgatggagagagatccacccgctggtagcatggtgctggaggctgacaggtggaggaggtggggcagggtctgtccgagtgcctagaggaagagtaaaccttccagagatgggggacccagaaggaagcgcagagtggggttgggggaaggggataccggtggtcagaagaaatttattaacagtggatgggataagtctgtgtctggagggatcctggtggaggcagaagggtcctgcctcacctggattctctcactccctccccagactgcagccgaaccctggtccctcctccacaatgtggcttctcctcactctctccttcctgctggcatccacaggtgaggtggccccaggagggggccaggtctgtgggagcaggtgcccccttcccaagcatgtctgggcccagtgatctgccagcccctacctcacccagagaccactaaagatccttccttcaccctccacctgtgccaatgtccctaagcccttaccgtcaggtgctggtgctgctgctctggagtcgctatgttgcctggggcctctcgctgcccacgacaaggaacacggtcctggggttacacaaacctgagctgagtcctggggcaaccgcttccttgctgtgtgtccttgagggaactgcttcacctctctgggcttcgaatgccttctctataagacagcacccacttgagacaataacagtgaggtctcaatagcataacagaggtaatatacatagcaagcattagacaagtgctgagaggccaacagcacagacagactccagcttgagtcccacacctgccactccctgtctcttacagggtctttgaggggattaaatgtggttgtgtgtgaggcagaagcataagcctggcccaggtagtgccccttcaggtgtgcaagccaggcacggtgcttagagcttacatacaacgtctatgtgtggtgggcaccaccgacctcatttgacaagggaaggggctgtggctcagagggacggccacaacatcaaggtcaccttgggtgtcaggcaaactccagattgaactcagctgccacacaccaagaaattaattgtaacctgatgcctctcttctggagaaattggggggtggactttcattaacgttctgccacaaatgaccctcactcctgggggcccctgagacccccacgcctccagcctcccctccggctctctctgtgcactcacctacctgcctcgcgcctgcctgctgcgcccagctggggcctccaccttcctctggcttggactggccaggtgcagcctcggtgcccagctgttcagcccgtaccctccgcccttcggaggacgacctcacccttcctttgttaagccccttgtccaccacatccgcattcccctggtctcacgggggcctttggcccagttcctgactgtgatggggagagtgtgggcatttggtctggctgtgcaaatcctgcccctgtgtgggtgggagtgtgcatggcttcaaccttcaggggatgcatccacattgcccagtggagaggggtcctggtcctgtgaccttgaatgtctctaatcatgtccttaagcataatgccattctgtgtgtgtgtgtgtgtgtgtgtgtgtacatgcacgtgtgcagtgggtatacaaggccctgtatgttcacatcctctccacatgcatgagccagatccccatatgtgaaacccaatcagtgactccacagatctggcttgggggctgatctagagatggataaatatgtcctgccctggctgcctctggcttcagctgcatgtctttgaccttgaatgcccagccccgtgtctgggtgctgccccagacagcaagtccacatctgagtgttggccttctgggttggtgtctgcagctctaactctacaaaatgtcttgtgggtgaatcacggttttaaccttgacttttttttgtttgtttggttttttttgagacggagtctcgctctgccgcccaagctggagttcagtggtgcaacctcagctcactgcaacctccgcctcccaggttcaagcaattctgtctctgcctcccgagtagctagaattacaggcacgcaccaccacgcccagctgatttttgtatttttatttatttatttatttattttttagtagagacgggatttcacgatgttggccaggctggtctcaaactcctgacctcaggtgatccacccacctcggccttggcctcccaaagtgctgggattacaggcgtgagccaccacacctggccaaccttgactatttattataggtaattctgtgcagatgtctgacttatgttggccatctccaggatggacctgaactttcacacgtatgtccctgtgactaaatccaggtgtcatttgcaaaaaacaactaatattattaagtagctaccagggctaggtatcactcaccatacatacacacatgcacacacacacatacacattcctacctcatccttacaacaatcttcattttacagatgaggaaacagaggcacagacaggtcgaataacttactcaaagtttcacagctagtacattcgaacccaggcttaaggacccatctttgtccagaccctgtatgcaagtgtctgtgacactggatgccaagactcacactagagatgttgaatttaggtctgaacaatatccaattctgtgtgtgtgtttgtgtgtgcatgtgtgtgtgtgtatgtattcatgtcttaaccatccatattcatatacacatatgaacatctgtgctgtgattctttttttttttttttttttttttttgagatggagtttcactcttgtcacccaggctggagtgcaatggagcaacctccgctcactgcaatctccgcctcccgggttcaagcgattttcctgcctcagcctccagagtagctgggattacaggcacccgccaccatgcccagctaattttttgtatttttgttagagacagggtttccccatattggccaggctggtctcgaactcctgacctcaggtgatccacccgcctcggcctcccaaagtgctgggattacaggcatgagccaccgtgcccagcctgtgctgtgattcttgaagctgcaacccatgtgcatgcaagtgaatttcagcttccagtcctgtccatagctgtacctaagtgtggaagctggatgtgcatgtatgcatgtccatgaccttgtatagccacatctgggactcatactgcacactgaatttggctgacatgtccagactctggggccaaggctgggtcacacatactgagtggccacatgcgtttgacgtctgtgacaatttggtgaccgtgaatgactggtttcaagtgaccacctgtctgaacctgtatccagtgcccctgtctccacccccaaccacagaggacttcttgccctctggtctgttccccttcctctctctcccagagtcttatagcaaatggggtgggggctagagttctggagaaaacaggcagcggttgtaaataaacaacagggcaggcggagcatggtggctcacacctgtaatcccagcactttgggaggctgaggcgggcagagcatttgaagtcagaagtttgagactacctggctaacatggtgagacctcgtctctactaaaaatacaaaaattagccaggtgtggtggcgggcacctcagctactcgggaggctgaggcaggaggatcacttgaacccaggaggcggaagttgcagtgagctgagatcatgccactgcactccagcctgggcaaaagagtgagactccgtctcaaaaacaacaacaacaacaaaacaaaaaacagggcagggtgtcttgagaagttaggggaaaggcataggcatatagtagttagggcagggtgcaaggaaggtgtaggaggcaatgtaaacgtccctgtcctcaggcatcctctaccccttctcttagcagcccaggatggtgacaagttgctggaaggtgacgagtgtgcaccccactcccagccatggcaagtggctctctacgagcgtggacgctttaactgtggcgcttccctcatctccccacactgggtgctgtctgcggcccactgccaaagccggtatgaaggcaggggctcagggtcctgagggagcctggttcggggggaagagctcctagatttgggggaagacggaggcagacgccagaactcctgggttctgaaagacgaggaggccggatgtcaagcccctgggttaggaaggagtgtgtgtttcaaagccttcgatctctgaaggaggaaggagaagactagttccagcttttgagcctcagttctagggatgtgagaatcctggattcggggacagaccaggagggggctgggagtagttggaggggatcgagttctaggagtgtgcctgacttcagactcgttggtccttgaggagcaggggctggaaccattggcttcagggtcttgggaaaaggtaatgggatgtcgagatttctaaagggtcgggagacctcgggttgcccactctttgatctttctgtcctctacttgcgggtaaccactggcccgcactccactggcgggaaaaccactcgcccgcacagcttcatgagagtgcgcctgggagagcacaacctgcgcaagcgcgatggcccagagcaactacggaccacgtctcgggtcattccacacccgcgctacgaagcgcgcagccaccgcaacgacatcatgttgctgcgcctagtccagcccgcacgcctgaacccccaggtgcgccccgcggtgctacccacgcgttgcccccacccgggggaggcctgtgtggtgtctggctggggcctggtgtcccacaacgagcctgggaccgctgggagcccccggtcacaaggtgcgtgaaaggatggagctggatgcgaggcctcaaggaatcctatgctccagggctcttgggcggaggggacaagggccggaatttatggatctgctccaagtccactgtcttccccagtgagtctcccagatacgttgcattgtgccaacatcagcattatctcggacacatcttgtgacaagagctacccagggcgcctgacaaacaccatggtgtgtgcaggcgcggagggcagaggcgcagaatcctgtgaggtcagagcctagaggggccatcaggcggaagaagagggatggggacaggtgtgggagtccggatggggttggattttctttgctttgggccagagaagatgctagggttaggcttggagatggagtaggaagagaagttagaataggggtgaggttggagttggggttataggtggggattgcgttgtttgaggtggataactgtgatagttagtttgagatggcatgggttggggttgagaatgggaatggtttggtttgattctgggtgggaaatacgtcagggttgaattgggatgaggtagattttgtttggaatgcagaagacatgaagattgagattggattttgagatgggcatgggtttgatttgattttgaatggtgaggatgtgggctgagttggatttaacttagtacagttgcactggagttgcatgggggtgagattggatataggttgggtgagttgtattgagctgtgttgaattggggttggggttggggttgggttggctctgtttgggataaactgggctgtattgagttgagttgggttggggttccctgggatggggatggattgggtttggggtgagattgcaaatggtgattaggatgaggatgaatccaggaggtttcactcaacctgagaccccctcttttccccacagggtgactctgggggacccctggtctgtgggggcatcctgcagggcattgtgtcctggggtgacgtcccttgtgacaacaccaccaagcctggtgtctataccaaagtctgccactacttggagtggatcagggaaaccatgaagaggaactgactattctagcctatctcctgtgcccctgactgagcagaagcccccacagctggccagcagccccgcctgacatggaacagaacggagccatcccccaagaccctgtccaaggcccagatgttagccaaggacttgtcccacctgaggacaaagctggcgctcaaggtcacctgtttaatgccaagataacaaagcgctgatccaagttgctctgtaggaatttctgtgacttttttctggggtcaaagagaaaccccgagacactgtacactgttccttttcacccaccaccccgatccctaggtgaggagaagcggcttgaagcagggctccattcattcaacacacatgaccacccgtgtgatcttgaacaagaggcccaatctcacttcgccttggtttccttatctgtaaaatgagaccatcttattgctgacttcaaagggctgttgtgaggattaaatgagatgattcgtctgaactgattaaaatcgtgtctggcactgagtaaataccctctatctctggatcccagttaaaggacctaacagacactagattaccaagaatggctttttctttaaggtttagttctgggccgggcatggtggctcacacctgtaatcccagcactttgggaggccaaggcgggcggctcacttgaggtcaggagtgcaagaccagcctggccaacatggtgaaaccccatctctactaaaaatactaaaaaaatttagccgggcgtggtggcacacgactgtaatcctagctacttgggagggtgatgtgggaggatcgcttgaacttaggaggcaggagttgcagtgagccgagatcgcgccactgcactccagcctggtgacagagcaagactccatctcagaaaaaaaaaaaaaaaaaaaaagatttagttctgggcttcctggtagccatggcaaaaaggcaaatactgtcctttccttagccaggtccctgatatacagcagaggctggaactctgagctgctttgattttaccaaaaagccaagacaacctgttggaagcctatgggtttaccattgaggctgcaggaatctagttcctaattatcttcagagaccacaaaatgtgatgttcaaggtcgctgaatgttgaagtacatgaacctggctcgtgagacctaaatattgtactggtggtgggggggaagggtcattggaatctgtggttagcctgatcttgacctgcgagggaaggttgtccagatctctggactttggaggaccgacgttgagcaccataatgggagcagaagtgcgaggtctttgagaccccgcttgttggggcggcgccggatttggatgctaaaaattacctgggaaccctgaatacatctgggttgggcgcacaatgtgtggctccccacacatctttaggaacacatttgggcaacccggtgggagtgaacggcctggc

SEQ ID NO: 12 - DNA, 20 bp, Artificial
  Primer
agtgggtcatcactgctgct

SEQ ID NO: 13 - DNA, 20 bp, Artificial
  Primer
tcgtttcctcaatccagctt

SEQ ID NO: 14 - DNA, 16 bp, homo sapiens
ggaggcaagcagcctc

SEQ ID NO: 15 - DNA, 20 bp, artificial
  Primer
tgtggcttctcctcactctc

SEQ ID NO: 16 - DNA, 18 bp, artificial
  Primer
aggctcgttgtgggacac

SEQ ID NO: 17 - DNA, 23 bp, artificial
  Primer
gaagctgaattcataattggtgg

SEQ ID NO: 18 - DNA, 21 bp, Artificial
  Primer
tttgttctagagctttgtccc

SEQ ID NO: 19 - protein, 14 aa, homo sapiens
SSQPWQAALLAGPR

SEQ ID NO: 20 - protein, 19 aa, Homo sapiens
AVRPIEVTQACASPGTSCR

SEQ ID NO: 21 - DNA, 1283 bp, homo sapiens
ccgcccccctcgtctctggtccctacctctctgctgtgtcttcatgtccctgagggtcttgggctctgggacctggccctcagcccctaaaatgttcctcctgctgacagcacttcaagtcctggctatagccatgacacagagccaagaggatgagaacaagataattggtggccatacgtgcacccggagctcccagccgtggcaggcggccctgctggcgggtcccaggcgccgcttcctctgcggaggcgccctgctttcaggccagtgggtcatcactgctgctcactgcggccgcccgatccttcaggttgccctgggcaagcacaacctgaggaggtgggaggccacccagcaggtgctgcgcgtggttcgtcaggtgacgcaccccaactacaactcccggacccacgacaacgacctcatgctgctgcagctacagcagcccgcacggatcgggagggcagtcaggcccattgaggtcacccaggcctgtgccagccccgggacctcctgccgagtgtcaggctggggaactatatccagccccatcgccaggtaccccgcctctctgcaatgcgtgaacatcaacatctccccggatgaggtgtgccagaaggcctatcctagaaccatcacgcctggcatggtctgtgcaggagttccccagggcgggaaggactcttgtcagggtgactctgggggacccctggtgtgcagaggacagctccagggcctcgtgtcttggggaatggagcgctgcgccctgcctggctaccccggtgtctacaccaacctgtgcaagtacagaagctggattgaggaaacgatgcgggacaaatgatggtcttcacggtgggatggacctcgtcagctgcccaggccctcctctctctactcaggacccaggagtccaggccccagcccctcctccctcagacccaggagtccaggcccccagcccctcctccctcagacccgggagtccaggcccccagcccctcctccctcagacccaggagccccagtccccagcatcctgatctttactccggctctgatctctcctttcccagagcagttgcttcaggcgttttctccccaccaagcccccacccttgctgtgtcaccatcactactcaagaccggaggcacagagggcaggagcacagaccccttaaaccggcattgtattccaaagacgacaatttttaacacgcttagtgtctctaaaaaccgaataaataatgacaataaaaatggaatcatcctaaattgta

SEQ ID NO: 22 - protein, 10 aa, Homo sapiens
IITGGHTCTR

SEQ ID NO: 23 - protein, 10 aa, Homo sapiens
QVTHPNYNSR

SEQ ID NO: 24 - protein, 11 aa, homo sapiens
QACASPGTSCR

SEQ ID NO: 25 - protein, 14 aa, Homo sapiens
SSQPWQAALLAGPR

SEQ ID NO: 26 - protein, 19 aa, Homo sapiens
AVRPIEVTQACASPGTSCR

SEQ ID NO: 27 - protein, 6 aa, artificial
  N-terminal recombinant hK14myc-His
EAEFII

SEQ ID NO: 28 - protein, 3 aa, artificial
  Peptide fragment of a Fluorogenic Synthetic Peptide
VPR

SEQ ID NO: 29 - protein, 4 aa, artificial
  Peptide Portion of Fluorogenic Synthetic Peptide
AAPF

SEQ ID NO: 30 - protein, 3 aa, Artificial
  Peptide Portion of Trypsin Substrate
VPR

SEQ ID NO: 31 - protein, 4 aa, Artificial
  Peptide Portion of Chymotrypsin Substrate
AAPF